(19) United States
(12) Patent Application Publication
(10) Pub. No.: US 2010/0009357 A1
(43) Pub. Date: Jan. 14, 2010

(54) PREDICTION OF LUNG CANCER TUMOR RECURRENCE

(76) Inventors: Joseph R. Nevins, Chapel Hill, NC (US); David Harpole, Chapel Hill, NC (US); Anil Potti, Chapel Hill, NC (US); Mike West, Durham, NC (US); Holly Dressman, Durham, NC (US)

Correspondence Address: MICHAEL BEST & FRIEDRICH LLP, 100 E WISCONSIN AVENUE, Suite 3300, MILWAUKEE, WI, 53202, US

(21) Appl. No.: 12/302,458

(22) PCT Filed: May 30, 2007
(86) PCT No.: PCT/US07/12685

§ 371 Date: Sep. 3, 2009

(30) Foreign Application Priority Data
May 30, 2006 (US) 60809702

(51) Int. Cl. C12Q 1/68

(52) U.S. Cl. 435/6

(57) ABSTRACT

The invention provides methods of estimating the likelihood of lung cancer recurrence in a subject, including those afflicted with NSCLC. The methods of the invention are useful for developing a therapeutic treatment plan to prevent cancer recurrence for subjects deemed to be at high risk, and withholding treatments from those subjects deemed to be at low risk. The invention also provides methods of generating and using metagene-based prediction tree models for estimating the likelihood of lung cancer recurrence. The invention also provides reagents, such as DNA microarrays, software and computer systems useful for estimating cancer recurrence, and provides methods of conducting a diagnostic business for the prediction of cancer recurrence.

### CROSS-REFERENCE TO RELATED APPLICATIONS

This application claims the benefit of the filing date of U.S. Application No. 60/809,702, filed May 30, 2006, entitled “PREDICTION OF LUNG CANCER TUMOR RECURRENCE.” The entire teachings of the referenced application are incorporated by reference herein.

### FIELD OF THE INVENTION

The field of this invention is cancer diagnosis and treatment.

### BACKGROUND OF THE INVENTION

Clinical trials have shown a benefit of adjuvant chemotherapy for patients diagnosed with Stage IB, II and IIIa non-small cell lung carcinoma. There has been no indication of benefit in Stage IA patients. This classification scheme is probably an imprecise predictor for the individual patient. Indeed, approximately 25% of Stage IA patients have a disease recurrence after surgery, suggesting the need to identify individuals in this subgroup for more effective therapy.

Lung cancer is the leading cause of cancer deaths worldwide. Non-small cell lung cancer accounts for approximately 80% of all disease cases (Cancer Facts and Figures, 2002, American Cancer Society, Atlanta, p. 11.). There are four major types of non-small cell lung cancer, including adenocarcinoma, squamous cell carcinoma, bronchoalveolar carcinoma, and large cell carcinoma. Adenocarcinoma and squamous cell carcinoma are the most common types of NSCLC based on cellular morphology (Travis et al., 1996, Lung Cancer Principles and Practice, Lippincott-Raven, New York, pps. 361-395). Adenocarcinomas are characterized by a more peripheral location in the lung and often have a mutation in the K-ras oncogene (Gazdar et al., 1994, Anticancer Res. 14:261-267). Squamous cell carcinomas are typically more centrally located and frequently carry p53 gene mutations (Niklinska et al., 2001, Folia Histochem. Cytobiol. 39:147-148).

The clinical staging system in NSCLC has been the standard for determining lung cancer prognosis. Although other clinical and biochemical markers have prognostic significance, the clinico-pathologic stage is believed to be the most accurate. The current standard of treatment for patients with stage I NSCLC is surgical resection, but nearly 30-35% of these patients will relapse after initial surgery. This relapse suggests that at least a subset of these patients might benefit from adjuvant chemotherapy. Similarly, patients with clinical stages Ib, IIa/IIb, and IIIa NSCLC, as a population, receive adjuvant chemotherapy. For some of these patients the potentially toxic chemotherapy is applied unnecessarily when surgical intervention would be adequate. The ability to more accurately stratify patients may therefore benefit health outcomes across the spectrum of disease.

Accordingly, a need remains for new methods of predicting and evaluating the need for adjuvant chemotherapy among patients afflicted with lung cancer and in particular with NSCLC. The invention provides these and related methods.

### SUMMARY OF THE INVENTION

The invention provides in part, an approach to risk stratification and treatment of NSCLC, using gene-expression patterns. These patterns more accurately estimate prognosis than previously possible, and can be used to identify patients with early-stage NSCLC at high risk for recurrence who would then be candidates for adjuvant chemotherapy.

The invention is based, in part, on the identification by Applicants of gene expression profiles that predicted risk the recurrence in a cohort of patients with early stage non-small cell lung carcinoma. The invention provides a prognostic model, named the Lung Metagene Predictor, capable of predicting the risk of recurrence of lung cancer in individual patients. The Lung Metagene Predictor is significantly better than clinical prognostic factors at predicting cancer recurrence. The improved prediction of recurrence may be observed, for example, at all the early clinical stages of NSCLC. In one embodiment, the Lung Metagene Predictor can identify a subset of Stage IA patients at higher risk of recurrence, who might in turn be best treated by adjuvant chemotherapy. In another embodiment, the Lung Metagene Predictor can identify a subset of Stage IB patients at lower risk of recurrence, to whom adjuvant chemotherapy may be withheld as a treatment.

One aspect of the invention provides a predictive model that uses a combination of clinical and genomic input variables to generate a predicted probability of cancer recurrence in NSCLC. In one embodiment, the models of the invention have the ability to predict NSCLC recurrence with a greater accuracy than is achievable using clinical parameters alone, such as when tested against an independent data set. One aspect of the invention provides methods of using predictive tree models having nodes that represent metagenes. The metagene for a cluster of genes is the dominant singular factor (principal component), computed using a singular value decomposition of expression levels of the genes in the metagene cluster on all samples. It represents the dominant average expression pattern of the cluster across tumor samples. In one embodiment, the cluster of gene contains at least 2, 3, 4, 5, 6, 7, 8, 9, 10, 12, 14, 16, 18, 20, 25, 30, 40, 50 or more genes. The set of metagenes and clinical factors may be used in binary classification tree analysis to recursively partition the samples into smaller subsets within which predictions of recurrence (0=5 year disease-free survival from diagnosis of recurrence, 1=death within 2.5 years from diagnosis of recurrence) are made in terms of estimated relative probabilities. The analysis computes and weighs many classification trees, and integrates them to provide overall risk predictions for each individual patient.

One aspect of the invention provides a method for predicting the likelihood of developing tumor recurrence in a subject afflicted with non-small cell lung cancer (NSCLC), the method comprising: (i) determining the expression level of multiple genes in a NSCLC sample from the subject; (ii) defining the value of one or more metagenes from the expression levels of step (i), wherein each metagene is defined by extracting a single dominant value using single value decomposition (SVD) from a cluster of genes associated with tumor recurrence; (iii) averaging the predictions of one or more statistical tree models applied to the values of the metagenes, wherein each model includes one or more nodes, each node representing a metagene, each node including a statistical predictive probability of tumor recurrence, thereby predicting the likelihood of developing tumor metastasis in a subject afflicted with non-small cell lung cancer (NSCLC). In one embodiment, the cluster of genes corresponding to at least one of the metagenes comprises 3, 4, 5, 6, 7, 8, 9 or 10 or more genes in common with metagene 19, 31, 35, 40, 41, 69, 74, 79 or 86, or a combination thereof. In one embodiment, the method comprises, prior to step (i), one of more of (1) providing the sample; (2) extracting, purifying or obtaining nucleic acids (such as mRNA) from the sample; (4) contacting the sample with an RNAse inhibitor; (5) contacting the sample with an aqueous solution; (6) removing the sample from the subject, such as through surgery; or (7) solubilizing nucleic acids (such as mRNA) contained in the sample.

One aspect of the invention provides a method for defining a statistical tree model predictive of NSCLC tumor recurrence, the method comprising: (i) determining the expression level of multiple genes in a set of non-small cell lung cancer samples, wherein the sample comprises samples from subjects with NSCLC recurrence and samples from subjects without NSCLC recurrence; (ii) identifying clusters of genes associated with metastasis by applying correlation-based clustering to the expression level of the genes; (iii) defining one or more metagenes, wherein each metagene is defined by extracting a single dominant value using single value decomposition (SVD) from a cluster of genes associated with NSCLC recurrence; and (iv) defining a statistical tree model, wherein the model includes one or more nodes, each node representing a metagene from step (iii), each node including a statistical predictive probability of NSCLC recurrence, thereby defining a statistical tree models predictive of NSCLC tumor recurrence. Step (iv) may be reiterated at least once to generate additional statistical tree models. In one embodiment, determining the expression level of multiple genes comprises determining the expression level of one or more mRNA gene products for each gene.

One aspect of the invention provides a computer-readable medium having computer-readable program codes embodied therein for performing binary prediction tree modeling to predict the recurrence of NSCLC based on gene expression data from the sample of a subject. In one embodiment, the computer-readable program codes performing functions comprises: (ii) defining the value of one or more metagenes from expression level values of multiple genes in the sample from the subject, wherein each metagene is defined by extracting a single dominant value using single value decomposition (SVD) from a cluster of genes associated with tumor recurrence; and (iii) averaging the predictions of one or more statistical tree models applied to the values of the metagenes, wherein each model includes one or more nodes, each node representing a metagene, each node including a statistical predictive probability of tumor recurrence.

One aspect of the invention provides a binary prediction tree modeling system for performing binary prediction tree modeling to predict the recurrence of NSCLC based on gene expression data from the sample of a subject. In one embodiment, the system comprises: (i) a computer; (ii) a computer-readable medium, operatively coupled to the computer, the computer-readable medium program codes performing functions comprising: (a) defining the value of one or more metagenes from expression level values of multiple genes in the sample from the subject, wherein each metagene is defined by extracting a single dominant value using single value decomposition (SVD) from a cluster of genes associated with tumor recurrence; (b) averaging the predictions of one or more statistical tree models applied to the values of the metagenes, wherein each model includes one or more nodes, each node representing a metagene, each node including a statistical predictive probability of tumor recurrence.

One aspect of the invention provides a method of conducting a diagnostic business that provides a health care practitioner with diagnostic information for the treatment of a subject afflicted with NSCLC. One such method comprises: (i) obtaining an NSCLC sample from the subject; (ii) determining the expression level of multiple genes in the sample; (iii) defining the value of one or more metagenes from the expression levels of step (ii), wherein each metagene is defined by extracting a single dominant value using single value decomposition (SVD) from a cluster of genes associated with tumor recurrence; (iv) averaging the predictions of one or more statistical tree models applied to the values, wherein each model includes one or more nodes, each node representing a metagene, each node including a statistical predictive probability of tumor recurrence, (v) providing the health care practitioner with the prediction from step (iv). The method optionally comprises one or more of the following steps: billing the subject, the subject's insurance carrier, the health care practitioner, or an employer of the health care practitioner; testing the sensitivity of an NSCLC cell from the subject to a chemotherapeutic agent; or determining if the subject carries an allelic form of a gene, such as of ras, EGFR or p53, whose presence correlates to sensitivity or resistance to a chemotherapeutic agent.

One aspect of the invention provides a computer-readable medium comprising a plurality of digitally-encoded values representing one or more sets of genes, wherein each set of genes corresponds to the cluster of genes defining a metagene, wherein the metagene is predictive of lung cancer recurrence in a statistical tree model. In one embodiment, at least 50%, 60%, 70%, 80%, 90% or 100% of the genes in each cluster are common to metagene 19, 31, 35, 40, 41, 69, 74, 79 or 86. The computer readable medium may optionally comprise computer-readable program codes embodied therein for performing binary prediction tree modeling to predict the recurrence of NSCLC based on gene expression data from the sample of a subject, the computer-readable medium program codes performing functions comprising: (ii) defining the value of one or more metagenes from expression level values of multiple genes in the sample from the subject, wherein each metagene is defined by extracting a single dominant value using single value decomposition (SVD) from one of the sets of genes; and (iii) averaging the predictions of one or more statistical tree models applied to the values of the metagenes, wherein each model includes one or more nodes, each node representing a metagene, each node including a statistical predictive probability of tumor recurrence.

One aspect of the invention provides a gene chip having a plurality of different oligonucleotides attached to a first surface of the solid support and having specificity for a plurality of genes, wherein at least 50% of the genes are common to those of metagenes 19, 31, 35, 40, 41, 69, 74, 79 and/or 86. In one embodiment, at least 60%, 70%, 80%, 90%, 95% or more of the genes are common to those of metagenes 19, 31, 35, 40, 41, 69, 74, 79 and/or 86.

One aspect of the invention provides a kit comprising any one of the gene chips provided herein and a computer-readable medium having computer-readable program codes embodied therein for performing binary prediction tree modeling to predict the recurrence of NSCLC based on gene expression data from the sample of a subject, the computer-readable medium program codes performing functions comprising: (ii) defining the value of one or more metagenes from expression level values of the plurality of genes, wherein each metagene is defined by extracting a single dominant value using single value decomposition (SVD) from a cluster of genes associated with tumor recurrence; (iii) averaging the predictions of one or more statistical tree models applied to the values of the metagenes, wherein each model includes one or more nodes, each node representing a metagene, each node including a statistical predictive probability of tumor recurrence.

### BRIEF DESCRIPTION OF THE FIGURES

FIGS. 1A-1E show the clinical and genomic prediction of risk of recurrence for NSCLC patients. FIG. 1A shows the scheme for development and validation of the lung prognosis model. FIG. 1B shows an example of one key metagene profile utilized in the recurrence risk prediction model. FIG. 1C shows an example of one classification tree illustrating incorporation of metagenes (mgene) at multiple levels to predict survival in the Duke cohort. Numbers and lines in red indicate patients who lived less than 2.5 years and blue numbers/lines represent patients with a greater than 5 year survival. The left box at each node of the tree identifies the number of patients, and the right box gives (as a percentage) the corresponding model-based point estimate of the 2.5-year recurrence probability based on the tree model predictions for that group. FIG. 1D shows predicted probability of recurrence based on the genomic model developed using the Duke cohort. Each patient is predicted in an out-of-sample cross validation based on a model completely regenerated from the data of the remaining patients. Red symbols (▴) indicate patients with recurrence and blue symbols (▪) indicate those without recurrence. FIG. 1E shows prediction of recurrence based on a clinical model. The left panel shows the probability of recurrence based on the clinical model generated using age, sex, tumor size, stage and smoking history. Each patient is predicted in an out-of-sample cross validation based on a model completely regenerated from the data of the remaining patients. Red symbols (▴) indicate patients with recurrence and blue symbols (▪) indicate those without recurrence.

FIGS. 2A-2B shows Kaplan Meier survival estimates based on genomic or clinical predictors. FIG. 2A shows Kaplan Meier survival curve estimates in the Duke cohort based on predictions from the genomic model demonstrate the increased value of the metagene approach. (p-values obtained using a log-rank test of significance). The red curve represents patients predicted to be high risk (>50% probability) of recurrence and the blue curve represents patients at low risk (≦50%) of recurrence. FIG. 2B shows Kaplan Meier survival curve estimates using the ‘clinical model’ of prognosis. The red curve represents patients predicted to be high risk (>50% probability) of recurrence and the blue curve represents patients at low risk (≦50%) of recurrence. Kaplan Meier survival estimates in the Duke cohort based on tumor size (T-size) or stage of disease are shown on the right.

FIGS. 3A-3B show independent validation of the lung metagene recurrence prediction model in the ACOSOG Z0030 and CALGB 9761 multi-institutional studies. FIG. 3A shows ACOSOG Z0030 validation. Left panel. The predictive model generated with the entire Duke set of samples was used to estimate recurrence probabilities for the ACOSOG samples. Red symbols (▴) indicate patients with recurrence and blue symbols (▪) indicate those without recurrence. Right panel. Kaplan Meier survival estimates by predictions of recurrence in the ACOSOG Z0030 cohort using the genomic model is shown. The red curve represents patients predicted to be high risk (>50% probability) of recurrence and the blue curve represents patients at low risk (≦50%) of recurrence. FIG. 3B shows CALGB 9761 validation. Left panel. The Duke predictive model was employed to predict the status of a set of 84 samples from the CALGB 9761 trial. Clinical outcomes were blinded to the investigators and predictive results were submitted to the CALGB statistical center for evaluation of performance. Red symbols (▴) indicate patients with recurrence and blue symbols (▪) indicate those without recurrence. Estimates of probability of recurrence along with 95% confidence intervals are shown. Right panel. Kaplan Meier survival estimates by predictions of recurrence in the CALGB 9761 cohort. The red curve represents patients predicted to be high risk (>50% probability) of recurrence and the blue curve represents patients at low risk (≦50%) of recurrence.

FIGS. 4A-4B show application of lung recurrence prediction model to refine assessment of risk and guide the use of adjuvant chemotherapy in Stage IA NSCLC. FIG. 4A shows Kaplan Meier survival curve estimates for all Stage IA patients (black curve) and those predicted at either high risk of recurrence (red) or low risk (blue) of recurrence. (For the purposes of this analysis, high risk of recurrence was defined as a greater than 50% probability of recurrence). FIG. 4B shows design of a planned prospective phase III clinical trial in patients with stage IA NSCLC to evaluate the performance of the genomic-based model of recurrence risk.

FIGS. 5A-5B show prediction of recurrence based on the genomic model as a function of NSCLC stage. FIG. 5A shows predictions of recurrence as a function of clinical stage. FIG. 5B shows Kaplan Meier estimates of survival by stage of NSCLC using the genomic model. The red curve represents patients predicted to be at high risk (>50% probability of recurrence) and the blue curve represents patients predicted to be at low risk (<50% probability of recurrence).

FIGS. 6A-6B show prediction of recurrence as a function of histological subtype. In FIG. 6A, red symbols indicate patients with recurrence and blue symbols indicate those without recurrence. FIG. 6B shows Kaplan Meier estimates of survival as a function of histological subtype.

FIG. 7 shows the performance of the metagene model to a previously published squamous NSCLC dataset (courtesy Dr. Zhifu Sun, Mayo Clinic). The predictive model generated with the entire Duke set of samples was used to estimate recurrence probabilities for the ACOSOG samples. Red symbols (▴) indicate patients with recurrence and blue symbols (▪) indicate those without recurrence.

FIG. 8 shows a block diagram of a computer system connected to a network according to an illustrative embodiment of the invention.

### DETAILED DESCRIPTION OF THE INVENTION

### I. Definitions

For convenience, certain terms employed in the specification, examples, and appended claims, are collected here. Unless defined otherwise, all technical and scientific terms used herein have the same meaning as commonly understood by one of ordinary skill in the art to which this invention belongs.

“Non-small cell lung cancer” refers to a cancer whose origin is in any of the cells of the lung except for those which are dedicated hormone-producing cells (e.g., the “small cells”).

The articles “a” and “an” are used herein to refer to one or to more than one (i.e., to at least one) of the grammatical object of the article. By way of example, “an element” means one element or more than one element.

The term “including” is used herein to mean, and is used interchangeably with, the phrase “including but not limited to”.

The term “or” is used herein to mean, and is used interchangeably with, the term “and/or,” unless context clearly indicates otherwise.

The term “such as” is used herein to mean, and is used interchangeably, with the phrase “such as but not limited to”.

“Lung cancer” refers in general to any malignant neoplasm found in the lung. The term as used herein encompasses both fully developed malignant neoplasms, as well as premalignant lesions. A “subject having lung cancer” is a subject who has a malignant neoplasm or premalignant lesion in the lungs.

As used herein, the terms “neoplastic cells”, “neoplasia”, “tumor”, “tumor cells”, “cancer” and “cancer cells”, (used interchangeably) refer to cells which exhibit relatively autonomous growth, so that they exhibit an aberrant growth phenotype characterized by a significant loss of control of cell proliferation (i.e., de-regulated cell division). Neoplastic cells can be malignant or benign. A metastatic cell or tissue means that the cell can invade and destroy neighboring body structures.

A “patient”, “subject” or “host” to be treated by the subject method may mean either a human or non-human animal.

The term “microarray” refers to an array of distinct polynucleotides or oligonucleotides synthesized or deposited on a substrate, such as paper, nylon or other type of membrane, filter, chip, glass slide, or any other suitable solid support.

### II. Methods of Predicting/Estimating the Likelihood of Tumor Recurrence

The Lung Metagene Predictor of the invention provides a mechanism to refine the estimation of an individual patient's risk for disease recurrence and thus guide the selection of the proper treatment, such as the use of adjuvant chemotherapy in early stage NSCLC. Specifically, based on the current established guidelines for treatment of NSCLC patients, this approach can be used to specifically re-classify a subset of Stage IA patients to receive adjuvant chemotherapy. In one embodiment, the Lung Metagene Predictor predicts NSCLC tumor recurrence with greater accuracy than clinical variables. Clinical variables include the age of the subject, gender of the subject, tumor size of the sample, stage of cancer disease, histological subtype of the sample and smoking history of the subject. Clinical variables may also include family history of lung cancer.

One aspect of the invention provides a method for predicting, estimating, aiding in the prediction of, or aiding in the estimation of, the likelihood of developing tumor recurrence in a subject. One method comprises (i) determining the expression level of multiple genes in a NSCLC sample from the subject; (ii) defining the value of one or more metagenes from the expression levels of step (i), wherein each metagene is defined by extracting a single dominant value using single value decomposition (SVD) from a cluster of genes associated with tumor recurrence; and (iii) averaging the predictions of one or more statistical tree models applied to the values, wherein each model includes one or more nodes, each node representing a metagene, each node including a statistical predictive probability of tumor recurrence.

In one embodiment, the diagnostic methods of the invention predict the likelihood of developing tumor recurrence with at least 70% accuracy. In another embodiment, the methods predict the likelihood of developing tumor recurrence with at least 80% accuracy. In another embodiment, the methods predict the likelihood of developing tumor recurrence with at least 85% accuracy. In another embodiment, the methods predict the likelihood of developing tumor recurrence with at least 90% accuracy. In another embodiment, the methods predict the likelihood of developing tumor recurrence with at least 70%, 80%, 85% or 90% accuracy when tested against a validation sample. In another embodiment, the methods predict the likelihood of developing tumor recurrence with at least 70%, 80%, 85% or 90% accuracy when tested against a set of training samples. In another embodiment, the methods predict the likelihood of developing tumor recurrence with at least 70%, 80%, 85% or 90% accuracy when tested on NSCLC Type IA samples, Type IB samples, or combinations thereof.

### (A) Tumor Sample

In one embodiment, the diagnostic methods of the invention comprise determining the expression level of genes in a tumor sample from the subject, preferably a lung tumor sample. In one embodiment, the sample is a Type IA NSCLC sample or a Type IB NSCLC sample. In another embodiment, the NSCLC is type Ia/Ib, IIa/IIb or IIIa. Tumors may be classified into classes using the World Health Organization classification criteria (See for example World Health Organization. Histological Typing of Lung Tumors. 2nd Ed. Geneva, World Health Organization, 1981; Travis W D et al. World Health Organization International Histological Classification of Tumors. Histological Typing of Lung and Pleural Tumors. 3rd Edition Springer-Verlag, 1999). In one embodiment, the sample from the subject is an adenocarcinoma, a squamous cell carcinoma, a bronchoalveolar carcinoma, a surgically-resected stage I squamous cell lung cancer or a large cell carcinoma. In one embodiment of the methods described herein, the method comprises the step of surgically removing a tumor sample from the subject, obtaining a tumor sample from the subject, or providing a tumor sample from the subject. In one embodiment, the sample contains at least 40%, 50%, 60%, 70%, 80% or 90% tumor cells, either relative to the total number of cells in the sample or relative to total mass or volume of the sample. In preferred embodiments, samples having greater than 50% tumor cell content are used. In one embodiment, the tumor sample is a live tumor sample. In another embodiment, the tumor sample is a frozen sample. In one embodiment, the sample is one that was frozen within less than 5, 4, 3, 2, 1, 0.75, 0.5. 0.25, 0.1, 0.05 or less hours following extraction from the patient. Preferred frozen sample include those stored in liquid nitrogen or at a temperature of about −80° C. or below.

### (B) Gene Expression

The expression of the genes may be determined using any method known in the art for assaying gene expression. Gene expression may be determined by measuring mRNA or protein levels for the genes. In a preferred embodiment, an mRNA transcript of a gene may be detected for determining the expression level of the gene. In some embodiments, the expression level of more than one transcript is determined, such as by using a probe that spans an area common to more than one transcript. Based on the sequence information provided by the GenBank™ database entries, the genes can be detected and expression levels measured using techniques well known to one of ordinary skill in the art. For example, sequences within the sequence database entries corresponding to polynucleotides of the genes can be used to construct probes for detecting mRNAs by, e.g., Northern blot hybridization analyses. The hybridization of the probe to a gene transcript in a subject biological sample can be also carried out on a DNA array. The use of an array is preferable for detecting the expression level of a plurality of the genes. As another example, the sequences can be used to construct primers for specifically amplifying the polynucleotides in, e.g., amplification-based detection methods such as reverse-transcription based polymerase chain reaction (RT-PCR). Furthermore, the expression level of the genes can be analyzed based on the biological activity or quantity of proteins encoded by the genes.

Methods for determining the quantity of the protein includes immunoassay methods. Paragraphs 98-123 of U.S. Patent Pub No. 2006-0110753 provide exemplary methods for determining gene expression. Additional technology that may be used in the present invention is described in U.S. Pat. Nos. 5,143,854; 5,288,644; 5,324,633; 5,432,049; 5,470,710; 5,492,806; 5,503,980; 5,510,270; 5,525,464; 5,547,839; 5,580,732; 5,661,028; 5,800,992 and in WO 95/21265; WO 96/31622; WO 97/10365; WO 97/27317; EP 373 203; and EP 785 280, the disclosures of which are all herein incorporated by reference.

In one exemplary embodiment, about 1-50 mg of lung cancer tissue is added to a chilled tissue pulverizer, such as to a BioPulverizer H tube [Bio101 Systems, Carlsbad, Calif.]. Lysis buffer, such as from the Qiagen Rneasy Mini kit, is added to the tissue and homogenized. Devices such as a Mini-Beadbeater [Biospec Products, Bartlesville, Okla.] may be used. Tubes may be spun briefly as needed to pellet the garnet mixture and reduce foam. The resulting lysate may be passed through syringes, such as a 21 gauge needle, to shear DNA. Total RNA may be extracted using commercially available kits, such as the Qiagen RNeasy Mini kit. The samples may be prepared and arrayed using Affymetrix U133 plus 2.0 GeneChips or Affymetrix U133A GeneChips.

In one embodiment, determining the expression level of multiple genes in a NSCLC sample from the subject comprises extracting a nucleic acid sample from the sample from the subject, preferably an mRNA sample. In one embodiment, the expression level of the nucleic acid is determined by hybridizing the nucleic acid, or amplification products thereof, to a DNA microarray. Amplification products may be generated, for example, with reverse transcription, optionally followed by PCR amplification of the products.

### (C) Genes Screened

In one embodiment, the diagnostic methods of the invention comprise determining the expression level of all the genes in the cluster that defines at least one lung-recurrence determinative metagene. For example, In one embodiment, the diagnostic methods of the invention comprise determining the expression level of at least 50%, 60%, 70%, 80%, 90%, 95%, 98%, 99% of the genes in each of the clusters that defines 1, 2, 3, 4 or 5 or more lung-recurrence determinative metagenes. In one embodiment, at least 50%, 60%, 70%, 80%, 90%, 95%, 98%, 99% of the genes whose expression levels are determined are genes represented by the following symbols: AARS, ABCA2, ABCF1, ABCF1, ABL2, ACADVL, ACLY, ACLY, ACLY, ACO2, ACTA2, ACTB, ACTN4, ACTL6A, ACTN1, ACTN1, ADAM8, ADAM10, ADAM10, ADCY7, ADD3, AP2A1, AP2B1, AP2B1, AHCY, AKT1, AKT2, ALAS2, ALDH1B1, ALDOA, ALPPL2, AMD1, AMD1, AMPD2, SLC25A5, ANXA1, ANXA5, ANXA6, ANXA7, APAF1, APLP2, APLP2, APP, ARAF, ARCN1, ARF1, ARF3, ARF4, ARF4, RHOA, RHOA, RHOB, RHOC, ARHGAP1, ARHGDIA, ARHGDIA, ARL1, ARL3, ARNT, ASMT, ASPA, ASPH, ATF4, ATM, ATM, RERE, RERE, RERE, ATP5G2, ATP5J, ATP6V1B1, ATP6V1B2, ATP6V0C, ATP6V0A1, ATP5O, KIF1A, BAI2, BARD1, BARD1, BCL2L1, BCL7A, BLVRA, BMP7, ZFP36L1, KLF5, BTG1, BTG1, SERPING1, C8orf1, ZNHIT2, PTTG1IP, TMEM50B, CALD1, CALM1, CALM1, CALM1, CALM3, CALM3, CALR, CALR, CALR, CALR, CALU, CALU, CALU, CALU, CAMK4, CANX, CANX, CAPG, CAPN1, CAPNS1, CAPNS1, CASP8, CAV2, CAV3, RUNX2, RUNX1, RUNX1, RUNX1, RUNX3, RUNX3, CBFB, CCKBR, CCND2, CCND2, CCND2, CCNG2, CD9, MS4A1, TNFSF8, SCARB2, CD58, CD59, CD59, CD59, CD63, CD81, CDC5L, CDC42, CDC42, CDH1, CDH1, CDK2, CDK4, CDKN2B, CENPB, CFL1, CTSC, CHD3, CHD4, CHML, CHRNE, CKB, CIRBP, AP2M1, TPP1, CLTA, CLTC, CNN3, COL6A1, COPA, KLF6, KLF6, SLC31A1, COX4I1, COX5B, COX6A1, COX7A2, COX7C, COX8A, CPD, CPE, CREB1, CREBL2, CSE1L, CSF1, CSF2RA, CSH2, CSNK1A1, CSNK1D, CSNK1E, CSRP1, CSTB, CTNNA1, CTSB, CTSD, CYC1, CYP1B1, CYP2A6, CYP2C9, CYP11B2, CYP27B1, DAF, DAP, DCTD, DCTN1, DDX3X, DDX5, DHX15, DEFA6, DHCR24, DLG1, DLG4, DMP1, DNASE1L2, DNASE2, DNMT2, DPYSL2, DR1, SLC26A3, DRG2, ATN1, TSC22D3, DSP, HBEGF, DUSP1, DUSP2, DUSP4, DUSP5, DUSP6, DYRK1A, EBF, ECH1, ECHS1, PHC2, EEF1A2, EEF1B2, EEF1D, EEF1G, EEF2, LGTN, EFNB1, EGR1, EIF1AX, EIF1AX, EIF1AX, EIF1AX, EIF2S1, EIF2S1, EIF2S1, EIF2S3, EPHA2, EIF4A1, EIF4A2, EIF4E, EIF4EBP2, EIF4G2, EIF5A, EIF5A, SERPINB1, ELF1, ELK4, EMP1, CTTN, CTTN, ENO1, ENO1, ENO2, ENSA, EPAS1, EPB41L1, STOM, STOM, STOM, EPHB1, EPHB2, EPHB3, EPHB3, EPOR, EPRS, EPRS, EPRS, ERBB3, EREG, ESRRA, EVX1, EXT1, EZH2, FANCA, FANCA, ACSL3, FARSLA, FAU, FEN1, FEN1, FGF5, FGF9, FGF11, FGFR3, FGFR2, FGFR2, FGFR4, FKBP1A, FKBP4, FKBP4, FKBP5, FOXE1, FOXO3A, FLI1, FLNA, FN1, FNTA, FNTA, FPR1, FTH1, FTHP1, FUS, FUS, FUT5, FUT7, FZD2, XRCC6, GAA, GABPA, GAD2, GAS6, GCH1, GDI1, B4GALT1, GLG1, GLO1, GLRB, GLUD1, GLUD1, GLUL, GM2A, GNA11, GNA11, GNA11, GNAI2, GNAI3, GNAI3, GNAI3, GNAL, GNAO1, GNAS, GNAS, GNAS, GNB1, GNB1, GNB1, GNB2, GNS, GNS, GOLGB1, GOLGB1, GOT2, GPR27, GPS1, GPX1, GPX4, GRN, GRIN2D, GRINA, NR3C1, CXCL1, CXCL2, CXCL3, GSN, GSTP1, GTF2I, GYPB, H1F0, H2AFZ, H3F3A, HADHB, HCFC1, HDAC1, HDGF, HDLBP, HFE, HIF1A, HINT1, HINT1, HK1, HLA-DOA, HLA-DPB1, HLA-E, HMGB1, HMGN1, HMGCR, HNRPA1, HNRPC, HNRPH1, HNRPH2, HNRPK, HNRPU, HPCA, HPGD, HPX, HRAS, HSBP1, HSBP1, HSD11B1, DNAJB2, DNAJA1, DNAJA1, HSPA1A, HSPA4, HSPA8, HSPA9B, HSPA9B, HSPA9B, HSPCA, HSPD1, HSPD1, DNAJB1, DNAJB1, IDH1, IFNGR1, IGF2R, IGFBP7, IGFBP7, IGHM, IK, IK, IL1A, IL1B, IL6ST, IL8, IL11, IL13RA2, INPP1, INPP4A, INSIG1, ITGA2, ITGB5, ITGB5, ITPR1, ITPR2, ITPR3, ITPR3, ITPR3, JUN, JUN, JUN, JUNB, JUND, JUP, KARS, KARS, KARS, KCNJ10, KCNK1, KCNK1, KCNN4, KIR2DL1, KLRC3, KNS2, KPNB1, KPNA2, TNPO1, KTN1, AFF3, LAIR1, LAMC1, LAMP1, LAMC2, LAMP2, STMN1, LASP1, LDHA, LDHB, LDLR, LDLR, LGALS1, LGALS3BP, LGALS8, LIF, ABLIM1, LIPA, LMAN1, LMAN1, LMNA, LMNB1, LNPEP, LPP, LRP1, LRP3, LRPAP1, LSS, LU, LYN, SH2D1A, M6PR, M6PR, M11S1, NBR1, MXD1, SMAD6, MAN2C1, MAN2A1, MAP4, MARK3, MAT1A, MAT2A, MAX, MAZ, MBNL1, MCL1, MCM2, MCM3, MCM4, MCP, MDH1, MDM4, ME2, MEF2A, MAP3K1, MET, MFAP2, MGAT1, MGST2, CD99, MID1, MAP3K11, MMP2, MMP14, MMP15, MNT, MPP3, MSH3, MSN, MST1R, MSX2, MUC1, MYB, MYC, MYD88, MYF6, MYH11, NACA, NARS, NASP, NBL1, NCL, NDP, NDUFB2, NDUFB8, NDUFS8, RPL10A, NFE2L1, NFE2L2, NFIB, NFIX, NFKB1, NFKB2, NFYA, NGFR, NHP2L1, NIT1, NMT1, NOL1, CNOT3, CNOT4, NPM1, NT5E, NTF5, NUCB1, NUMA1, NUMA1, OAS2, ODC1, OGG1, P2RX4, P4HB, P4HB, PA2G4, PAFAH1B1, SERPINB2, PALM, PARN, PC, PCBP2, PCNA, PCTK2, PDE8A, PDHA1, PEX12, PF4, PFDN4, PFKFB3, PFKL, PFKM, PFKP, PFN1, PFTK1, PGAM1, PGD, PGK1, PGM1, PGM5, PHB, PHB, SLC25A3, PHF1, PHF2, SERPINB9, PIK3CG, PIK4CB, PIN1, PITPNA, PKD1, PKM2, PKNOX1, PLAU, PLAUR, PLCB3, PLEC1, PLEK, PLOD1, PLP2, PLXNA2, PMM2, PMS2, POLD2, POLE2, POLR2A, PPGB, PPIB, PPM1A, PPM1G, PPP1CA, PPP1CC, PPP2R1A, PPP2R4, PPP2R5E, PPP3R2, PPP6C, PPT1, PRG1, PRKAB1, PRKAR1A, PRKCA, PKN2, PRKCSH, MAPK1, MAP2K7, MAP2K7, PRL, PRPS1, PRPS2, HTRA1, PSAP, PSMA7, PSMB1, PSMB2, PSMB4, PSMB7, PSMC2, PSMD1, PSMD2, PSMD4, PSMD8, PSME1, PTBP1, PTGER3, PTGS1, PTK9, PTMA, PTPN11, PTPN12, PTPRF, PTX3, PURA, PVR, PXMP2, PXN, PYGB, RAB1A, RAB5A, RAB6A, RAB6A, RABSC, RAB5C, RAD17, RAD21, RAD23A, RAD51C, RAN, RAP1B, RARG, JARID1A, RBBP7, RBL2, RBM4, RCN1, RENT1, RFC3, RFX5, RHEB, BRD2, RPA1, RPA3, RPL4, RPL4, RPL4, RPL5, RPL6, RPL7, RPL8, RPL9, RPL10, RPL10, RPL12, RPL12, RPL12, RPL17, RPL17, RPL18, RPL18, RPL19, RPL27, RPL27, RPL28, RPL29, RPL31, RPL31, RPL32, RPL37, RPL37, RPLP0, RPLP0, RPLP1, RPLP2, RPLP2, RPN1, RPS3A, RPS3A, RPS4X, RPS5, RPS5, RPS6, RPS6, RPS7, RPS7, RPS8, RPS10, RPS10, RPS10, RPS10, RPS11, RPS11, RPS13, RPS14, RPS15, RPS15A, RPS18, RPS19, RPS20, RPS21, RPS23, RPS25, RPS25, RPS27, RPS27A, RPS29, RRBP1, RRBP1, RRBP1, RRBP1, RREB1, RRM1, RRM2, RSU1, S100A5, S100A10, S100A11, S100A13, SAA4, SAFB, SARS, SAT, SCD, SCD, SCD, SCD, SCD, SCN8A, SCNN1A, CCL16, SDC1, SDCBP, SDHA, SDHC, SDHD, SEL1L, SEL1L, SEPW1, SET, SET, SFRS2, SFRS2, SFRS2, SFRS3, SFRS6, SFRS6, SFRS7, SFRS7, SFRS8, SFRS10, SFRS10, SH3BGR, SIL, SKP1A, SKP1A, SKP1A, SKP2, SLC1A4, SLC1A7, SLC2A3, SLC3A2, SMTN, SMTN, SLC5A3, SLC9A1, SLC12A2, SLC12A4, SLC34A1, SLC20A1, SLC22A2, SMARCC1, SMPD2, SUMO3, SUMO3, SUMO2, SNRP70, SNRPD2, SOD1, SON, SON, SON, SORL1, SOX4, SOX12, SP1, SP2, UAP1, SPARC, SPG7, SPTBN1, SRC, SRP14, SRPR, SSB, SSFA2, SSR1, SSR1, SSR2, SSR3, SSR4, SSRP1, SSRP1, STAT1, STK4, STRN, VAMP1, VAMP2, TACC1, TAF7, TAF10, CNTN2, TBXA2R, TCEB2, TCEB2, TCF7L2, MLX, PRDX2, TEGT, TEP1, NR2F1, NR2F2, TGFB1I1, TGM1, TGM2, TGM2, THBS1, THP0, TIA1, TIMP3, TIMP3, TK2, TLE3, TLR1, TM7SF1, TMPO, TNFAIP1, TNFAIP1, TNFAIP3, TNFAIP3, TOP1, TOP1, TP53, TPD52, TPI1, TRA1, CCT3, TSN, TUBA1, TUFM, TXN, TYROBP, UBB, UBB, UBC, UBE1, UBE2B, UBE2D1, UBE2D3, UBE2G2, UBE2H, UBE2L3, UBE2L3, UBE2L3, UBE2L3, UBE2N, UBE3A, UGCG, UGT8, UPP1, UQCRC2, UTRN, UTX, VCL, VDAC1, VEGF, VEGF, VEGF, VIL2, VMD2, VRK1, VRK2, WARS, WARS, WNT5A, WNT5A, WNT5A, XBP1, XIST, XPNPEP2, YY1, YWHAE, ZNF3, ZNF207, ZNF207, SLC30A1, MAP3K12, ZYX, PTP4A1, PTP4A1, PTP4A1, PTP4A1, LRP8, TUBA3, USP7, DEK, ALDH5A1, BAT1, BAT1, JTV1, JTV1, JTV1, RASSF7, RASSF7, FOSL1, PTP4A2, MLF2, MLL2, FXR1, PABPN1, PABPN1, ANP32A, C16orf35, SLC7A5, SF3A2, GDF5, LZTR1, USP9X, SLC10A3, BRAP, FZD1, FZD1, PIP5K2B, PIP5K2B, SLC25A11, SPARCL1, SPOP, TAGLN2, CUL4B, CUL4B, CUL1, SMARCA5, ARGBP2, PPFIA1, KCNAB2, CSDA, CSDA, CPZ, BCAS1, API5, AGPS, LMO4, CGGBP1, AP3B1, BHLHB2, BHLHB2, PIAS1, PIAS1, TCAP, CDK10, PRPF18, D21S2056E, MKNK1, KHSRP, SLC25A12, SLC25A12, PPAP2B, VDP, CDC2L5, DNCL10, EIF3S10, EIF3S10, EIF3S10, EIF3S8, EIF3S7, EIF3S5, EIF3S5, STX16, STX16, BECN1, BECN1, PEA15, PEA15, HYAL2, TRADD, PABPC4, RAB11A, RAB11A, SNAP23, SNAP23, CREG1, FGF18, INPP4B, IQGAP1, IQGAP1, NRP2, NRP1, CD84, CFLAR, CFLAR, CFLAR, CFLAR, WISP1, KSR, IER3, VNN1, TAX1BP1, MCM3AP, PRPF4B, CCNA1, APIS2, SCAP2, H1FX, WASL, ATP6V0E, MPZL1, RPL14, GPCR5A, GPRC5A, SLC7A6, SLC7A6, PAPSS2, PAPSS1, TBX19, FCGR2C, SLC16A3, FAM50A, RNU3IP2, SYNGR2, CTDP1, SFRS2IP, EDG4, OSMR, BUB3, LRRFIP1, BMP15, NOLC1, NOLC1, LRAT, DLG5, RPS6KA5, MFHAS1, MFHAS1, PSCD2, PSCD1, COPB2, SFRS11, SFRS11, B4GALT6, CNOT8, VAMP3, RPL23, SLC9A3R1, TM9SF2, LIPG, RECQL5, C1orf38, ONECUT2, PSMF1, PSMF1, LITAF, LITAF, SPTLC2, GDF15, NPEPPS, NPEPPS, TMEM59, TP53I3, RAB3D, SEC22L1, SEC22L1, CDC42BPB, PRDX6, PRDX6, WTAP, AKAP12, IER2, PDIA4, NCOR1, NCOR1, NCOR1, NCOR1, NUP155, ZNF592, PDE4DIP, ZNF432, EIF5B, EIF5B, EIF5B, EIF5B, KIAA0406, ENTH, BZW1, PUM1, PUM1, PUM1, KIAA0100, LAPTM4A, KIAA0152, KIAA0152, KIAA0195, BCLAF1, BCLAF1, BCLAF1, TM9SF4, MATR3, MATR3, SNX17, DLG7, SPCS2, KIAA0174, DAZAP2, DAZAP2, TOMM20, TOMM20, KIAA0494, GIT2, DNAJC6, TRIM 14, PSF1, KIAA0528, PJA2, SEC24D, ZC3H11A, KIAA0196, G3BP2, G3BP2, MFN2, KIAA0020, ARHGAP25, WDR1, WDR1, SLC23A2, FGFBP1, ROD1, ACOT8, TANK, BCL2L11, FRAT1, RANBP9, UBA2, FARSLB, C21orf6, PQBP1, PQBP1, ARPC5, ACTR3, ACTR2, TSPAN3, ACTR1A, BCAP31, MBNL2, TRIM28, RCL1, LHFPL2, TNK2, SDCCAG33, SDCCAG33, PSME3, PSME3, CALCRL, EIF1, HNRPR, RABEPK, STUB1, SAP18, PAK4, UNG2, B3GALT5, NOC4, K-ALPHA-1, ISGF3G, ANAPC10, NDRG1, MYL9, GNB2L1, ST3GAL6, YAP1, SPON1, ZYG11BL, MAP3K7IP1, HAX1, GPNMB, HMGN4, HMGN4, SEC23B, CAP1, SYNCRIP, COVA1, SEMA6B, DDX17, CHERP, HYOU1, IPO7, NOL5A, RNASEH2A, DCTN2, TM9SF1, ARL6IP5, ARPC1A, CCT7, CCT4, CCT2, NPC2, USP16, CDC42EP3, PAICS, PDLIM5, TRIM3, SPFH1, HIS1, TGOLN2, TXNIP, KHDRBS1, B3GNT1, CCT8, MGEA5, NUDC, PTGES3, STAG2, RAI2, MAP3K2, GIPC1, AHCYL1, FUSIP1, RPP40, UTP14A, PPP1R13L, ASE-1, CD3EAP, PGRMC1, BLCAP, TRAFD1, RNPS1, EHD1, SMAP, KDELR1, HNRPA0, SEC61B, TDE1, OS9, TMED2, LMAN2, RAB40B, CKAP4, TMED10, IMMT, SF3B2, GLIPR1, TLK2, KDELR2, LILRA2, LILRA2, DSTN, TRIOBP, C9orf7, HNRPUL1, FAF1, PWP1, PSIP1, WDHD1, STRAP, PTENP1, AKAP10, RPL35, CA5B, CHP, DDX19, PARK7, FKBP9, CBX3, CBX3, GABARAP, XRN2, MRAS, RASA3, DLGAP4, DLGAP4, AAK1, NALP1, SEC31L1, Cep164, MAPRE1, SEPHS2, RAB18, AKR7A3, FBXO21, CNOT1, CNOT1, KIAA0992, TMCC1, JMJD2B, KIAA1117, SMG1, PEG10, ARHGAP26, CDC2L6, TNRC6B, PARC, MAP3K7IP2, JMJD3, KIAA0543, CLCC1, GPD1L, KIAA0217, UBXD2, CYFIP1, C9orf10, KIAA0280, XTP2, MAST4, SCC-112, KIAA0460, ATP11A, ANKRD12, KIAA0802, ZC3H7B, EXOC7, TSPYL4, KIAA0367, FBXW11, C17orf31, ACSL6, USP22, SMCHD1, KIAA0323, MECT1, DULLARD, DICER1, RHOQ, TARDBP, HARSL, SF3B3, SF3B1, TRAM1, CAPN7, BRD4, PES1, SKIV2L2, RPL13A, RPL13A, SRRM2, CLCF1, ARL2BP, TMEM50A, SH3BP1, BP75, PLD3, SSBP3, TMEFF2, C9orf5, OSBP2, IL17R, FKBP8, MTCH1, FBXO7, PGLS, PGLS, LMOD1, LSM4, TNFAIP8, NIPBL, RAB26, DKFZP586A0522, ZNF473, RCHY1, CCDC28A, RIS1, COBRA1, GEMIN5, CLIC4, CLIC4, DKFZP564G2022, TBC1D10B, NELF, DKFZP434O047, HERC4, TOR1AIP1, C1orf144, WSB1, IRF2BP1, GTPBP5, B3GAT3, FBXO9, VPS33B, EHF, GNL3, PTPN18, SLC17A5, FER1L3, DAZAP1, PCOLCE2, NUFIP1, AKAP8L, TCL6, C2orf24, HTF9C, GHITM, SERP1, AHDC1, ZNF330, RAB30, MAC30, PCLO, DLL1, GIT1, PRO1073, ATAD2, PRO0478, BTBD15, METTL5, HSPC182, SSU72, TMOD3, TMOD2, CARD10, REPIN1, ALG5, ANAPC2, STRN4, TRA2A, EPN1, SEC61A1, PKN3, TAX1BP3, MINK1, COL5A3, CHST11, IPLA2(GAMMA), C6orf48, TMED5, SLC35C2, EXOSC1, HDDC2, MRPS18C, LAP3, CGI-07, TXNDC14, ABHD5, SH3GLB1, PHF20L1, DREV1, IER31P1, LOC51136, ZNF580, DCTN4, LEF1, NIN, CRIM1, PAIP2, ANKMY1, BM88, MSCP, MRPL35, WAC, FZR1, HOOK1, TEX264, CRLF3, TRPV2, ANAPC5, SFMBT1, EPLIN, HSPC148, DTL, NCKIPSD, CINP, RAB14, RAB14, UFM1, PIAS4, DHRS7, TMBIM4, BIT1, ZFR, TMEM66, OAZ3, CAB39, CAB39, CROP, ARTS-1, ZAK, MBD3, C21orf45, TERF2IP, ETAA16, NLE1, DGCR8, FLJ10404, MNAB, GNL3L, EPB41L4B, PD2, TBC1D13, GTPBP2, PPM2C, FEV, FBLIM1, C10orf92, AHI1, NDE1, APTX, FLJ20254, EPS8L1, BCOR, BCOR, FLJ20345, RPP25, IMPAD1, IMPAD1, TMEM70, C20orf27, TUG1, C22orf8, FLJ10154, FLJ10159, SLC6A15, SLC6A15, C6orf166, FLJ10661, ATAD3A, SMU1, FLJ10815, PSPC1, PHF10, FLJ11301, FLJ11301, PRO1580, PRO1843, MEG3, MEG3, MEG3, MCM10, HSA277841, KIAA1704, H41, VEZATIN, ANKRD10, C20orf42, TRMT1, PNRC2, HIF1AN, SCYL2, DSU, PACS1, FRMD4A, LUC7L, RPRC1, NECAP2, ODZ3, TMEM30A, WDR12, NGLY1, DDX28, LRP2BP, UBAP2, C3orf10, THEM2, C20orf19, TPARL, HT007, WSB2, ZNF302, MLL5, ZNF313, C1orf91, ANKH, YLPM1, BCCIP, PEO1, ZC3HAV1, C1orf119, DKFZP434H132, STARD7, EXOSC5, DUSP22, DC12, XAB2, CCNL1, TWSG1, DDX24, DDX24, KLP1, PHF22, PHTF2, C15orf17, C20orf74, THOC2, VANGL2, SNX14, CSRP2BP, PBXIP1, CBX8, REXO1, KIAA1205, ODF2L, ARID1B, HEG, MTA3, MTUS1, XPO5, CGN, TAOK1, TRMT5, KIAA1543, KIAA1553, C17orf27, CHD8, KIAA1602, KIAA1967, ZNF410, CTDSP1, OVOL2, PRUNE, ZNF462, DC2, SR-A1, C19orf29, RBM25, RRAGD, MESDC1, CDH26, SPCS3, SCOC, 11P45, BCORL1, E2-230K, ELMO2, MRPS14, FLJ22965, MCCC2, LIN7B, DIO3OS, OSGEPL1, TOR3A, RHBDF1, NOC3L, NFKBIZ, MMP25, NARFL, HIAT1, TMPRSS3, NUCKS, PDIA2, ACBD3, C20orf81, FLJ22318, MICAL1, CERK, CYP3A43, KLC2, FBXL17, RBM21, CDCP1, MRPS5, C2orf23, ACD, RAPH1, RTN4R, NOL6, MARCKSL1, PLEKHA3, PHACTR4, ALS2CR3, MGC5242, MGC2803, PRRG4, SLC25A23, C9orf16, C20orf149, ZSCAN5, GDPD3, LENG1, MGC10433, MGC11256, C1orf89, ZBED2, FLJ12684, SAP30L, ZYG11B, PRKRIP1, FLJ23436, TBC1D17, FLJ13639, C5orf14, ZNF408, CXorf45, FLJ21148, PRG2, FLJ11783, GRHL2, CXorf34, PCNXL2, FLJ21918, C10orf97, PANK2, FLJ12595, FLJ13111, FLJ21128, CHD9, C1orf22, MTERFD3, EFHD1, MED28, FLAD1, CPEB4, ULBP2, PRO2730, CYB5-M, CMIP, CMIP, ZFP91, TXNDC, FBXO38, YIPF5, MAP1LC3B, C6orf62, C6orf62, TRIM7, NETO2, NETO2, C20orf55, YPEL3, KCTD10, HDAC10, TM2D1, BBP, TMPRSS13, C1orf160, C9orf81, CHD6, DKFZp434F142, MAF1, ANKRD32, MGC10854, MGC13186, MGC14595, DOT1L, USP38, PLA2G12B, PLA2G12B, N-PAC, PPP1R9B, NYD-SP20, MGC10955, ZNF577, MGC11324, LMNB2, MINA, TBRG1, CIRH1A, ZNRF1, C9orf37, COL27A1, COL27A1, COL27A1, SHANK3, MADP-1, KIAA1754, SSH2, PNPT1, NAV3, FCHSD1, SAMD1, YIF1B, LOC90799, LASS5, C19orf6, UAP1L1, BTF3L4, UBXD5, ACY3, YT521, MGC13138, TIFA, ZNF651, OLFM2, ARHGAP12, FOXQ1, H2AFV, MRLC2, MGC16943, BTBD14B, SCAMP4, RHPN1, LENG8, C1QTNF7, KCTD12, KCTD12, PCMTD1, MGC24381, KLHDC3, C6orf192, CENTB5, SSX2IP, C10orf104, TMEM45B, TTC8, SLC25A29, C16orf55, NHN1, LOC124402, FLJ30656, ALDH16A1, C19orf28, HSPB6, C1orf93, TMEM77, OACT2, FLJ30834, MGC29898, NUDCD2, LOC134492, APXL2, ACY1L2, ZNF358, NEK7, C20orf96, C20orf112, BRI3BP, D1c2, SFRS12, LOC144097, PRICKLE1, FLJ32549, TOM1L2, RTN4RL1, C1orf51, MANEAL, FLJ35801, DAB2IP, IRX2, LOC153914, OACT1, CAMSAP1, RASEF, LOC158160, LOC162073, DENND2C, FLJ37927, GLIS3, RP13-15M17.2, SPRED2, KIAA2018, LOC220074, OTUD1, EFHA1, C6orf89, LOC221955, TMED4, C6orf69, ZBTB38, FLJ35740, ZDHHC20, KCTD13, LOC255783, FRMD3, LOC257407, NCR3, BCL9L, 15E1.2, C13orf8, KIAA0220, FLJ90652, LOC283922, LOC284058, LOC284112, LOC284184, LOC285148, ZNF707, C4orf10, MMAB, LOC339745, FLJ34283, LOC348120, HILS1, C9orf111, AGRN, LOC388554, FLJ16518, LOC390998, TTMB, LOC399491, LOC400642, FLJ37798, FLJ34077, CTXN1, MIRN21, LOC440151, LOC440983, C11orf32, KTN1, PDIA6, TRAPPC2, SEDLP, UTP14C, UTP14A, YWHAQ, MIB1, NUDT4, NUDT4P1, ADH1A, ADH1B, ADH1C, KIAA1245, LOC200030, MGC8902, BZW1, LOC151579, PML, LOC161527, DJ328E19.C1.1, FLJ20719, LOC200030, MGC8902, AE01, AG1, LOC440675, FLJ20719, LOC200030, MGC8902, AE01, AG1, LOC440675, GOLGA8A, GOLGA8B, ARHGAP8, LOC553158, FLJ46061, RPS28, PCDHGC3, PCDHGB4, PCDHGA8, PCDHGA12, PCDHGC5, PCDHGC4, PCDHGB7, PCDHGB6, PCDHGB5, PCDHGB3, PCDHGB2, PCDHGB1, PCDHGA11, PCDHGA10, PCDHGA9, PCDHGA7, PCDHGA6, PCDHGA5, PCDHGA4, PCDHGA3, PCDHGA2, PCDHGA1, PCDHGC3, PCDHGB4, PCDHGA8, PCDHGA12, PCDHGC5, PCDHGC4, PCDHGB7, PCDHGB6, PCDHGB5, PCDHGB3, PCDHGB2, PCDHGB1, PCDHGA11, PCDHGA10, PCDHGA9, PCDHGA7, PCDHGA6, PCDHGA5, PCDHGA4, PCDHGA3, PCDHGA2, PCDHGA1, WASL, LOC441150, RPL7L1, GTF2I, GTF2IP1, H3F3A, LOC440926, H3F3A, LOC440926, HSPA1A, HSPA1B, NPIP, LOC339047, LOC440341, EIF3S5, LOC339799, RPL34, LOC342994, RPL34, LOC342994, IGH, IGHD, IGHG1, LOC349338, FLJ25222, MGC52000, IMAA, LOC388221, LOC440345, LOC440354, LOC595101 and LOC641298.

In one embodiment, the expression level of additional genes—which do not correspond to a lung-recurrence determinative metagene or which do not correspond to the genes that define metagenes 19, 31, 35, 40, 41, 69, 74, 79 or 86—may also be determined. In one embodiment, the gene whose expression is determined is not an EGFR-RS gene, an RYK gene, a TNFRSF25 gene, a TRPM7 gene, an UNC5H2 gene, a KCP3 gene or a KIAA1883 gene. Sequences for these genes are disclosed in U.S. Patent Pub. No. 2006/0110753.

### (D) Subjects

The subject is preferably a mammal. In some embodiments, the mammal is a nonhuman mammal. In another embodiment, the mammal is a human. In one embodiment, the subject is a non-human primate, mouse, rat, dog, cat, horse and cow. The subjects may include those afflicted with non-small cell lung cancer (NSCLC). Subjects afflicted with NSCLC include those presently having lung cancer (e.g. carry a lung tumor), as well as those who have had a lung tumor removed, such as through surgery. In one embodiment, the subject is one who has been diagnosed with lung cancer within 10, 9, 8, 7, 6, 5, 4, 3, 2, 1, 0.5, or 0.0825 years from the time the diagnostic method is to be applied. In one preferred embodiment, the lung cancer that the subject is afflicted with, or that has been afflicted with, is NSCLC. In one preferred embodiment, the NSCLC that the subject is afflicted with, or that has been afflicted with, is Type IA NSCLC or Type IA NSCLC. In one embodiment, the NSCLC that the subject is afflicted with, or that has been afflicted with, is type Ia/Ib, IIa/IIb or IIIa NSCLC. In one embodiment, the subject is afflicted with, or has been afflicted with, lung cell adenocarcinoma, lung squamous cell carcinoma, stage I squamous cell lung cancer or with a lung large cell carcinoma. In one preferred embodiment, the subject is afflicted with, or has been afflicted with, lung cell adenocarcinoma or lung squamous cell carcinoma or both. In one embodiment, the subject is a male. In one embodiment, the subject is a female. In one embodiment, the subject is a smoker. In one embodiment, the subject is not a smoker.

### (E) Metagene Valuation

In one embodiment, the diagnostic methods of the invention comprise defining the value of one or more metagenes from the expression levels of the genes. A metagene value is defined by extracting a single dominant value from a cluster of genes associated with tumor recurrence, preferably associated with NSCLC tumor recurrence. In a preferred embodiment, the dominant single value is obtained using single value decomposition (SVD). In one embodiment, the cluster of genes of each metagene or at least of one metagene comprises at least 3, 4, 5, 6, 7, 8, 9, 10, 12, 15, 18, 20 or 25 genes. In one embodiment, the diagnostic methods of the invention comprise defining the value of 2, 3, 4, 5, 6, 7, 8, 9 or 10 or more metagenes from the expression levels of the genes.

In preferred embodiments of the methods described herein, at least 1, 2, 3, 4, 5, 6, 7, 8 or 9 of the metagenes is metagene 19, 31, 35, 40, 41, 69, 74, 79 or 86. In one embodiment, at least one of the metagenes comprises 3, 4, 5, 6, 7, 8, 9 or 10 or more genes in common with any one of metagenes 19, 31, 35, 40, 41, 69, 74, 79 or 86. In one embodiment, a metagene shares at least 50%, 60%, 70%, 80%, 90%, 95%, 98%, 99% of the genes in its cluster in common with a metagene selected from 19, 31, 35, 40, 41, 69, 74, 79 or 86.

In one embodiment, the diagnostic methods of the invention comprise defining the value of 2, 3, 4, 5, 6, 7, 8 or more metagenes from the expression levels of the genes. In one embodiment, the cluster of genes from which any one metagene is defined comprises at least 3, 4, 5, 6, 7, 8, 9, 10, 11, 12, 13, 14, 15, 16, 17, 18, 19, 20, 22 or 25 genes.

In one embodiment, the diagnostic methods of the invention comprise defining the value of at least one metagene wherein the genes in the cluster of genes from which the metagene is defined, shares at least 50%, 60%, 70%, 80%, 90%, 95% or 98% of genes in common to any one of metagenes 19, 31, 35, 40, 41, 69, 74, 79 or 86. In one embodiment, the diagnostic methods of the invention comprise defining the value of at least two metagenes, wherein the genes in the cluster of genes from which each metagene is defined shares at least 50%, 60%, 70%, 80%, 90%, 95% or 98% of genes in common to anyone of metagenes 19, 31, 35, 40, 41, 69, 74, 79 or 86. In one embodiment, the diagnostic methods of the invention comprise defining the value of at least three metagenes, wherein the genes in the cluster of genes from which each metagene is defined shares at least 50%, 60%, 70%, 80%, 90%, 95% or 98% of genes in common to anyone of metagenes 19, 31, 35, 40, 41, 69, 74, 79 or 86. In one embodiment, the diagnostic methods of the invention comprise defining the value of at least four metagenes, wherein the genes in the cluster of genes from which each metagene is defined shares at least 50%, 60%, 70%, 80%, 90%, 95% or 98% of genes in common to anyone of metagenes 19, 31, 35, 40, 41, 69, 74, 79 or 86. In one embodiment, the diagnostic methods of the invention comprise defining the value of at least five metagenes, wherein the genes in the cluster of genes from which each metagene is defined shares at least 50%, 60%, 70%, 80%, 90%, 95% or 98% of genes in common to anyone of metagenes 19, 31, 35, 40, 41, 69, 74, 79 or 86. In one embodiment, the diagnostic methods of the invention comprise defining the value of a metagene from a cluster of genes, wherein at least 2, 3, 4, 5, 6, 7, 8, 9, 10, 11, 12, 13, 14, 15, 16, 17, 18, 19 or 20 genes in the cluster are selected from any one of Tables 1-9.

In one embodiment, at least one of the metagenes is metagene 19, 31, 35, 40, 41, 69, 74, 79 or 86. In one embodiment, at least two of the metagenes are selected from metagenes 19, 31, 35, 40, 41, 69, 74, 79 or 86. in one embodiment, at least three of the metagenes are selected from metagenes 19, 31, 35, 40, 41, 69, 74, 79 or 86. In one embodiment, at least three of the metagenes are selected from metagenes 19, 31, 35, 40, 41, 69, 74, 79 or 86. In one embodiment, at least four of the metagenes are selected from metagenes 19, 31, 35, 40, 41, 69, 74, 79 or 86. In one embodiment, at least five of the metagenes are selected from metagenes 19, 31, 35, 40, 41, 69, 74, 79 or 86.

In one embodiment of the methods described herein, one of the metagenes whose value is defined (i) is metagene 19 or (ii) shares at least 2, 3, 4, 5, 6, 7, 8, 9, 10, 11, 12 or 13 genes in common with metagene 19. In one embodiment of the methods described herein, one of the metagenes is defined by at least 2, 3, 4, 5, 6, 7, 8, 9 or all of genes in the following set: HPGD, RARG, SLC10A3, PEX12, LAF4, EREG, PF4, NIPBL, DEFA6 and SH2D1A. Table 1 shows the cluster of genes that defines metagene 19.

TABLE 1
Genes in the Cluster Defining Metagene 19

| | | Gene |
|---|---|---|
| ProbeSet ID | Gene Title | Symbol |
| 200908_s_at | — | — |
| 203914_x_at | hydroxyprostaglandin dehydrogenase | HPGD |
| | 15-(NAD) | |
| 204189_at | retinoic acid receptor, gamma | RARG |
| 204928_s_at | solute carrier family 10 (sodium/bile acid | SLC10A3 |
| | cotransporter family), member 3 | |
| 205094_at | peroxisomal biogenesis factor 12 | PEX12 |
| 205734_s_at | lymphoid nuclear protein related to AF4 | LAF4 |
| 205767_at | epiregulin | EREG |
| 206390_x_at | platelet factor 4 (chemokine (C—X—C | PF4 |
| | motif) ligand 4) | |
| 207108_s_at | Nipped-B homolog (Drosophila) | NIPBL |
| 207572_at | — | — |
| 207814_at | defensin, alpha 6, Paneth cell-specific | DEFA6 |
| 211211_x_at | SH2 domain protein 1A, Duncan's disease | SH2D1A |
| | (lymphoproliferative syndrome) | |
| 213443_at | — | — |
| 213873_at | — | — |

In one embodiment of the methods described herein, one of the metagenes whose value is defined (i) is metagene 31 or (ii) shares at least 2, 3, 4, 5, 6, 7, 8, 9, 10, 11 or 12 genes in common with metagene 31. In one embodiment of the methods described herein, one of the metagenes is defined by at least 2, 3, 4, 5, 6, 7, 8, 9, 10, 11, 12 or all of genes in the following set: RPS21, PFKP, FXR1, CAPG, ATP5J, RPS6KA5, WDHD1, FEV, EFHD1, CCKBR, EXOC7, EFHA1 and UQCRC2. Table 2 shows the cluster of genes that defines metagene 31.

TABLE 2
Genes in the Cluster Defining Metagene 31

| | | Gene |
|---|---|---|
| ProbeSet ID | Gene Title | Symbol |
| 200834_s_at | ribosomal protein S21 | RPS21 |
| 201037_at | phosphofructokinase, platelet | PFKP |
| 201637_s_at | fragile X mental retardation, | FXR1 |
| | autosomal homolog 1 | |
| 201850_at | capping protein (actin filament), gelsolin-like | CAPG |
| 202325_s_at | ATP synthase, H+ transporting, | ATP5J |
| | mitochoridrial F0 complex,subunit F6 | |
| 204633_s_at | ribosomal protein S6 kinase, 90 kDa, | RPS6KA5 |
| | polypeptide 5 | |
| 204727_at | WD repeat and HMG-box DNA binding | WDHD1 |
| | protein 1 | |
| 207260_at | FEV (ETS oncogene family) | FEV |
| 209343_at | EF hand domain family, member D1 | EFHD1 |
| 210381_s_at | cholecystokinin B receptor | CCKBR |
| 212034_s_at | exocyst complex component 7 | EXOC7 |
| 212410_at | EF hand domain family, member A1 | EFHA1 |
| 212600_s_at | ubiquinol-cytochrome c reductase core | UQCRC2 |
| | protein II | |

In one embodiment of the methods described herein, one of the metagenes whose value is defined (i) is metagene 35 or (ii) shares at least 2, 3 or 4 genes in common with metagene 35. In one embodiment of the methods described herein, one of the metagenes is defined by at least 2, 3, 4 or all of genes in the following set: HMGCR, LMOD1, FOXE1, EPHB2 and TRA2A. Table 3 shows the cluster of genes that defines metagene 35.

TABLE 3
Genes in the Cluster Defining Metagene 35

| | | Gene |
|---|---|---|
| ProbeSet ID | Gene Title | Symbol |
| 202539_s_at | 3-hydroxy-3-methylglutaryl-Coenzyme A | HMGCR |
| | reductase | |
| 203766_s_at | leiomodin 1 (smooth muscle) | LMOD1 |
| 206912_at | forkhead box E1 (thyroid | FOXE1 |
| | transcription factor 2) | |
| 211165_x_at | EPH receptor B2 | EPHB2 |
| 213575_at | Transformer-2 alpha | TRA2A |

In one embodiment of the methods described herein, one of the metagenes whose value is defined (i) is metagene 40 or (ii) shares at least 2, 3, 4, 5, 6, 7, 8, 9, 10, 11, 12, 13, 14, 15, 16, 17, 18, 19, 20, 21, 22, 23, 24 or 25 genes in common with metagene 40. In one embodiment of the methods described herein, one of the metagenes is defined by at least 2, 3, 4, 5, 6, 7, 8, 9, 10, 11, 12, 13, 14, 15, 16, 17, 18, 19, 20, 21, 22, 23, 24 or all of genes in the following set: ABCF1, DNAJA1, GNAS, IPO7, CPE, PGRMC1, SSB, NMT1, CHD4, NPEPPS, ACTL6A, SSX2IP, MSX2, NUDT4, EPOR, CAMK4, CYP3A43, RPLP0, ZNF339, AMPD2, YLPM1, SCAMP4, MUC1, ABHD5 and CYP2C9. Table 4 shows the cluster of genes that defines metagene 40.

TABLE 4
Genes in the Cluster Defining Metagene 40

| | | Gene |
|---|---|---|
| ProbeSet ID | Gene Title | Symbol |
| 200045_at | ATP-binding cassette, sub-family F (GCN20), member 1 | ABCF1 |
| 200881_s_at | DnaJ (Hsp40) homolog, subfamily A, member 1 | DNAJA1 |
| 200981_x_at | GNAS complex locus | GNAS |
| 200995_at | Importin 7 | IPO7 |
| 201116_s_at | carboxypeptidase E | CPE |
| 201120_s_at | progesterone receptor membrane component 1 | PGRMC1 |
| 201138_s_at | Sjogren syndrome antigen B (autoantigen La) | SSB |
| 201159_s_at | N-myristoyltransferase 1 | NMT1 |
| 201182_s_at | chromodomain helicase DNA binding protein 4 | CHD4 |
| 201455_s_at | aminopeptidase puromycin sensitive | NPEPPS |
| 202666_s_at | actin-like 6A | ACTL6A |
| 203018_s_at | synovial sarcoma, X breakpoint 2 interacting protein | SSX2IP |
| 205556_at | msh homeo box homolog 2 (Drosophila) | MSX2 |
| 206302_s_at | nudix (nucleoside diphosphate linked moiety X)-type motif 4 | NUDT4 |
| 209963_s_at | erythropoietin receptor | EPOR |
| 210349_at | calcium/calmodulin-dependent protein kinase IV | CAMK4 |
| 211442_x_at | cytochrome P450, family 3, subfamily A, polypeptide 43 | CYP3A43 |
| 211444_at | — | — |
| 211720_x_at | ribosomal protein, large, P0 ribosomal protein, large, P0 | RPLP0 |
| 211778_s_at | zinc finger protein 339 zinc finger protein 339 | ZNF339 |
| 212360_at | adenosine monophosphate deaminase 2 (isoform L) | AMPD2 |
| 212787_at | YLP motif containing 1 | YLPM1 |
| 213244_at | secretory carrier membrane protein 4 | SCAMP4 |
| 213693_s_at | Mucin 1, transmembrane | MUC1 |
| 213935_at | abhydrolase domain containing 5 | ABHD5 |
| 214421_x_at | cytochrome P450, family 2, subfamily C, polypeptide 9 | CYP2C9 |

In one embodiment of the methods described herein, one of the metagenes whose value is defined (i) is metagene 41 or (ii) shares at least 2, 3, 4, 5, 6, 7, 8, 9, 10, 11, 12, 13, 14 or 15 genes in common with metagene 41. In one embodiment of the methods described herein, one of the metagenes is defined by at least 2, 3, 4, 5, 6, 7, 8, 9, 10, 11, 12 or all of genes in the following set: ARAF, MGST2, VNN1, RAD51C, SLC26A3, PIK3CG, JTV1, ALPPL2, TP53I3, CPZ, MINA, KPNB1 and PCBP2. Table 5 shows the cluster of genes that defines metagene 41.

TABLE 5
Genes in the Cluster Defining Metagene 41

| | | Gene |
|---|---|---|
| ProbeSet ID | Gene Title | Symbol |
| 201895_at | v-raf murine sarcoma 3611 viral oncogene | ARAF |
| | homolog | |
| 204168_at | microsomal glutathione S-transferase 2 | MGST2 |
| 205844_at | vanin 1 vanin 1 | VNN1 |
| 206066_s_at | RAD51 homolog C (S. cerevisiae) | RAD51C |
| 206143_at | solute carrier family 26, member 3 | SLC26A3 |
| 206370_at | phosphoinositide-3-kinase, catalytic, gamma | PIK3CG |
| | polypeptide | |
| 207737_at | — | — |
| 209971_x_at | JTV1 gene | JTV1 |
| 210431_at | alkaline phosphatase, placental-like 2 | ALPPL2 |
| 210609_s_at | tumor protein p53 inducible protein 3 | TP53I3 |
| 211062_s_at | carboxypeptidase Z carboxypeptidase Z | CPZ |
| 211369_at | — | — |
| 213188_s_at | MYC induced nuclear antigen | MINA |
| 213507_s_at | karyopherin (importin) beta 1 | KPNB1 |
| 213517_at | Poly(rC) binding protein 2 | PCBP2 |
| 214207_s_at | — | — |

In one embodiment of the methods described herein, one of the metagenes whose value is defined (i) is metagene 69 or (ii) shares at least 2, 3, 4, 5, 6, 7, 8, 9, 10, 11, 12 or 13 genes in common with metagene 69. In one embodiment of the methods described herein, one of the metagenes is defined by at least 2, 3, 4, 5, 6, 7, 8, 9, 10, 11, 12 or all of genes in the following set: RFX5, LOC153914, SLC31A1, DNMT2, PDIP, KCNJ10, PRKCA, IL11, FLJ46061, SYNCRIP, HARSL, PTBP1, TLK2 and CA5B. Table 6 shows the cluster of genes that defines metagene 69.

TABLE 6
Genes in the Cluster Defining Metagene 69

| | | Gene |
|---|---|---|
| ProbeSet ID | Gene Title | Symbol |
| 202964_s_at | regulatory factor X, 5 (influences HLA | RFX5 |
| | class II expression) | |
| 203969_at | hypothetical protein LOC153914 | LOC153914 |
| 203971_at | solute carrier family 31 (copper | SLC31A1 |
| | transporters), member 1 | |
| 206308_at | DNA (cytosine-5-)-methyltransferase 2 | DNMT2 |
| 206691_s_at | protein disulfide isomerase, pancreatic | PDIP |
| 206692_at | potassium inwardly-rectifying channel, | KCNJ10 |
| | subfamily J, member 10 | |
| 206923_at | protein kinase C, alpha | PRKCA |
| 206924_at | interleukin 11 | IL11 |
| 208902_s_at | FLJ46061 protein | FLJ46061 |
| 209024_s_at | synaptotagmin binding, cytoplasmic RNA | SYNCRIP |
| | interacting protein | |
| 209252_at | histidyl-tRNA synthetase-like | HARSL |
| 212016_s_at | polypyrimidine tract binding protein 1 | PTBP1 |
| 212986_s_at | tousled-like kinase 2 | TLK2 |
| 214082_at | carbonic anhydrase VB, mitochondrial | CA5B |

In one embodiment of the methods described herein, one of the metagenes whose value is defined (i) is metagene 74 or (ii) shares at least 2, 3, 4, 5, 6, 7, 8, 9 or 10 genes in common with metagene 74. In one embodiment of the methods described herein, one of the metagenes is defined by at least 2, 3, 4, 5, 6, 7, 8, 9, 10, 11, 12 or all of genes in the following set: KIF1A, PALM, MSH3, MPP3, SAA4, DKFZP434O047, H3F3A, C1orf38, THPO and GOLGIN-67. Table 7 shows the cluster of genes that defines metagene 74.

TABLE 7
Genes in the Cluster Defining Metagene 74

| ProbeSet ID | Gene Title | Gene Symbol |
|---|---|---|
| 203850_s_at | kinesin family member 1A | KIF1A |
| 203859_s_at | paralemmin | PALM |
| 205887_x_at | mutS homolog 3 (E. coli) | MSH3 |
| 206186_at | membrane protein, palmitoylated 3 | MPP3 |
| | (MAGUK p55 subfamily member 3) | |
| 207096_at | serum amyloid A4, constitutive | SAA4 |
| 208008_at | DKFZP434O047 protein | DKFZP434O047 |
| 208755_x_at | H3 histone, family 3A | H3F3A |
| 210650_s_at | — | — |
| 210785_s_at | chromosome 1 open reading | C1orf38 |
| 211154_at | thrombopoietin (myeloproliferative | THPO |
| | leukemia virus frame 38 oncogene | |
| | ligand, megakaryocyte growth and | |
| | development factor) | |
| 213650_at | golgin-67 | GOLGIN-67 |

In one embodiment of the methods described herein, one of the metagenes whose value is defined (i) is metagene 79 or (ii) shares at least 2, 3, 4, 5, 6, 7, 8, 9, 10, 11, 12, 13, 14, 15, 16, 17 or 18 genes in common with metagene 79. In one embodiment of the methods described herein, one of the metagenes is defined by at least 2, 3, 4, 5, 6, 7, 8, 9, 10, 11, 12, 13, 14, 15, 16 or all of genes in the following set: CD59, PYGB, INSIG1, GAA, BCL7A, VRK1, NDP, CSH2, DRPLA, C6orf80, FZD2, NRP2, KIR2DL1, PRPF4B, RENT1, ACSL6 and MFHAS1. Table 8 shows the cluster of genes that defines metagene 79.

TABLE 8
Genes in the Cluster Defining Metagene 79

| | | Gene |
|---|---|---|
| ProbeSet ID | Gene Title | Symbol |
| 200983_x_at | CD59 antigen p18-20 (antigen identified by monoclonal | CD59 |
| | antibodies 16.3A5, EJ16, EJ30, EL32 and G344) | |
| 201481_s_at | phosphorylase, glycogen; brain | PYGB |
| 201627_s_at | insulin induced gene 1 | INSIG1 |
| 202812_at | glucosidase, alpha; acid (Pompe disease, glycogen storage | GAA |
| | disease type II) | |
| 203796_s_at | B-cell CLL/lymphoma 7A | BCL7A |
| 203856_at | vaccinia related kinase 1 | VRK1 |
| 205118_at | — | — |
| 206022_at | Norrie disease (pseudoglioma) | NDP |
| 206986_at | — | — |
| 208341_x_at | chorionic somatomammotropin hormone 2 | CSH2 |
| 208871_at | dentatorubral-pallidoluysian atrophy (atrophin-1) | DRPLA |
| 209479_at | chromosome 6 open reading frame 80 | C6orf80 |
| 210220_at | frizzled homolog 2 (Drosophila) | FZD2 |
| 210842_at | neuropilin 2 | NRP2 |
| 210890_x_at | killer cell immunoglobulin-like receptor, two domains, long | KIR2DL1 |
| | cytoplasmic tail, 1 | |
| 211090_s_at | PRP4 pre-mRNA processing factor 4 homolog B (yeast) | PRPF4B |
| | PRP4 pre-mRNA processing factor 4 homolog B (yeast) | |
| 211168_s_at | regulator of nonsense transcripts 1 | RENT1 |
| 211207_s_at | acyl-CoA synthetase long-chain family member 6 | ACSL6 |
| 213457_at | malignant fibrous histiocytoma amplified sequence 1 | MFHAS1 |

In one embodiment of the methods described herein, one of the metagenes whose value is defined (i) is metagene 86 or (ii) shares at least 2, 3, 4, 5, 6, 7, 8, 9, 10, 11, 12, 13 or 14 genes in common with metagene 86. In one embodiment of the methods described herein, one of the metagenes is defined by at least 2, 3, 4, 5, 6, 7, 8, 9, 10, 11, 12, 13, 14, 15 or all of genes in the following set: ADCY7, TYROBP, LRP3, SIL, SLC1A7, ARHGAP12, KLRC3, BMP7, TRAPPC2, MEG3 LOC440199, HFE, FKBP9, KIAA0650, LOC257407 and ARL3. Table 9 shows the cluster of genes that defines metagene 86.

TABLE 9
Genes in the Cluster Defining Metagene 86

| ProbeSet ID | Gene Title | Gene Symbol |
|---|---|---|
| 203741_s_at | adenylate cyclase 7 | ADCY7 |
| 204122_at | TYRO protein tyrosine kinase binding protein | TYROBP |
| 204381_at | low density lipoprotein receptor-related protein 3 | LRP3 |
| 205339_at | TAL1 (SCL) interrupting locus | SIL |
| 207355_at | solute carrier family 1 (glutamate transporter), member 7 | SLC1A7 |
| 207606_s_at | Rho GTPase activating protein 12 | ARHGAP12 |
| 207723_s_at | killer cell lectin-like receptor subfamily C, member 3 | KLRC3 |
| 209590_at | Bone morphogenetic protein 7 (osteogenic protein 1) | BMP7 |
| 209751_s_at | trafficking protein particle complex 2 | TRAPPC2 |
| 210794_s_at | maternally expressed 3 hypothetical gene supported by | MEG3 LOC440199 |
| | BX161452 | |
| 211326_x_at | hemochromatosis | HFE |
| 212169_at | FK506 binding protein 9, 63 kDa | FKBP9 |
| 212579_at | KIAA0650 protein | KIAA0650 |
| 213143_at | hypothetical protein LOC257407 | LOC257407 |
| 213433_at | ADP-ribosylation factor-like 3 | ARL3 |

In one embodiment, the clusters of genes that define each metagene are identified using supervised classification methods of analysis previously described (See West, M. et al. Proc Natl Acad Sci USA 98, 11462-11467 (2001)). The analysis selects a set of genes whose expression levels are most highly correlated with the classification of tumor samples into tumor recurrence versus no tumor recurrence. The dominant principal components from such a set of genes then defines a relevant phenotype-related metagene, and regression models assign the relative probability of tumor recurrence.

(F) Predictions from Tree Models

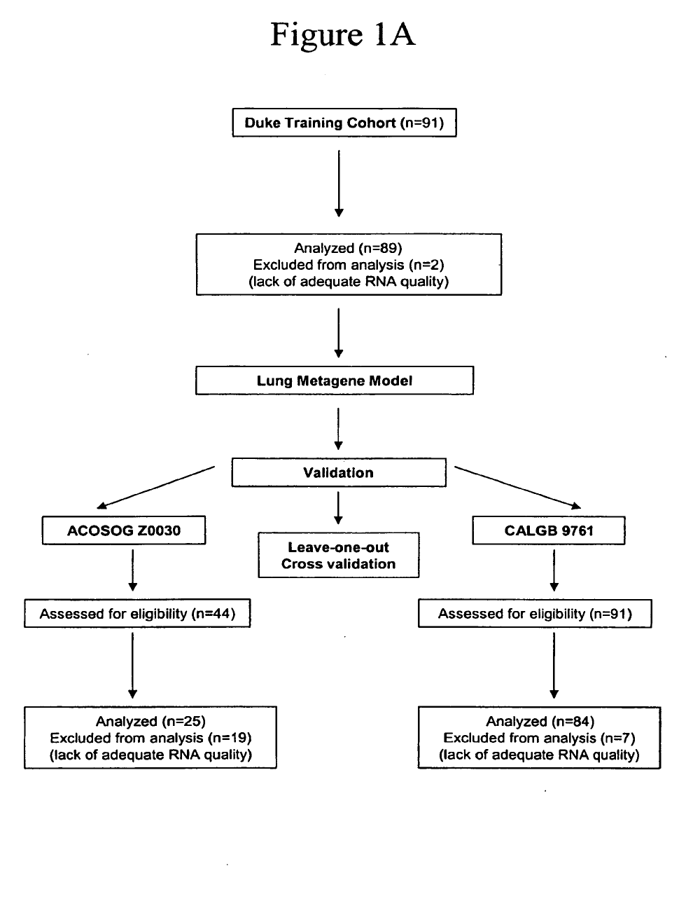

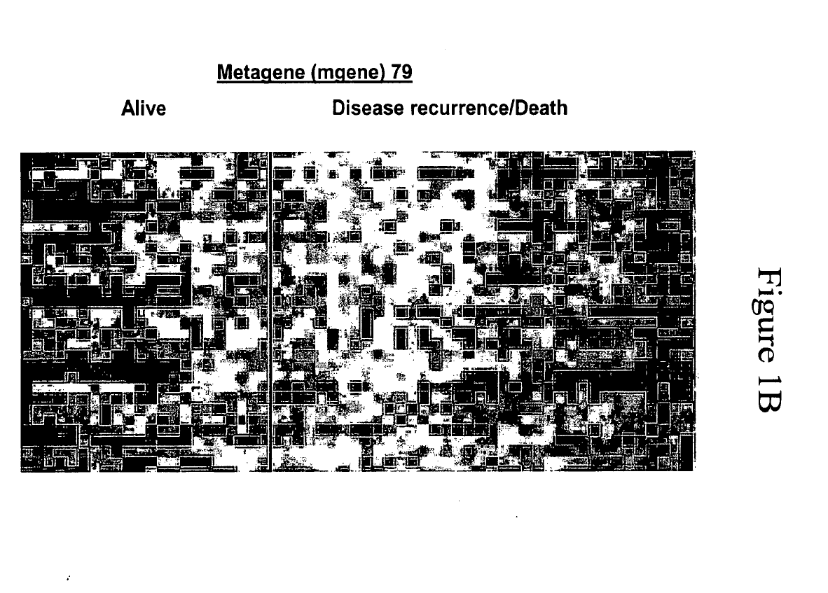

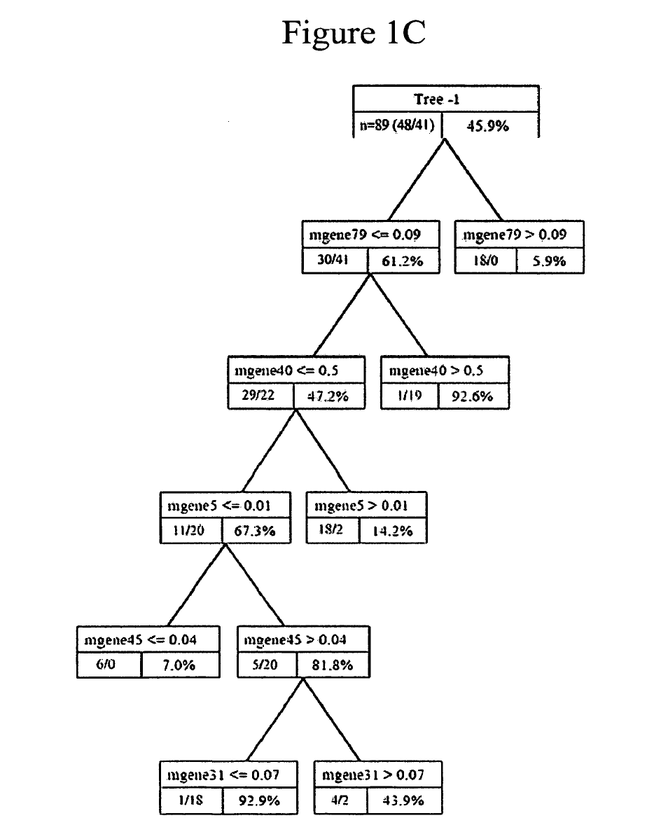

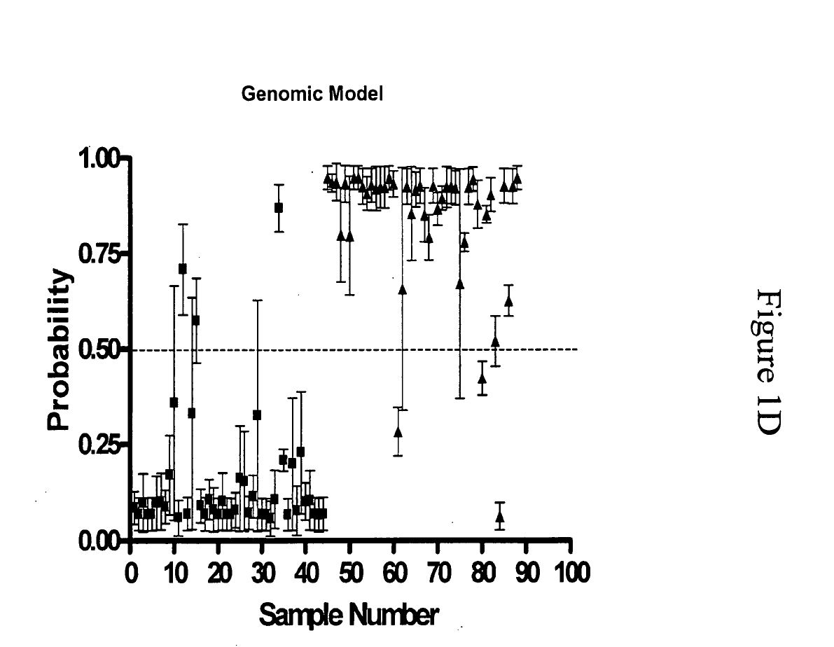

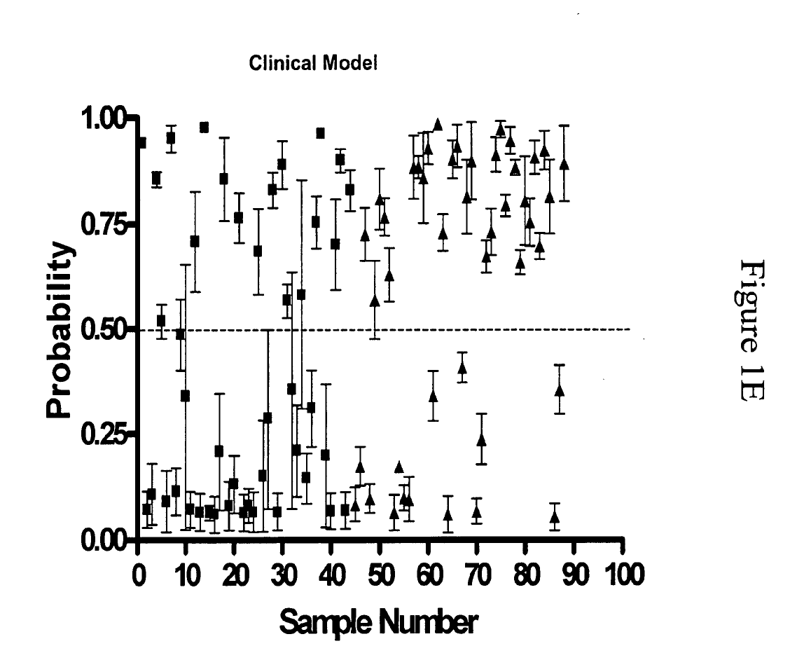

In one embodiment, the diagnostic methods of the invention comprise averaging the predictions of one or more statistical tree models applied to the metagenes values, wherein each model includes one or more nodes, each node representing a metagene, each node including a statistical predictive probability of tumor recurrence. FIG. 1 shows an exemplary statistical tree model that may be used in the methods described herein. The statistical tree models may be generated using the methods described herein for the generation of tree models. General methods of generating tree models may also be found in the art (See for example Pitman et al., Biostatistics 2004;5:587-601; Denison et al. Biometrika 1999;85:363-77; Nevins et al. Hum Mol Genet 2003;12:R153-7; Huang et al. Lancet 2003;361:1590-6; West et al. Proc Natl Acad Sci USA 2001;98:11462-7; U.S. Patent Pub. Nos. 2003-0224383; 2004-0083084; 2005-0170528; 2004-0106113; and U.S. application Ser. No. 11/198,782).

In one embodiment, the diagnostic methods of the invention comprise deriving a prediction from a single statistical tree model, wherein the model includes one or more nodes, each node representing a metagene, each node including a statistical predictive probability of tumor recurrence. In a preferred embodiment, the tree comprises at least 2 nodes. In a preferred embodiment, the tree comprises at least 3 nodes. In a preferred embodiment, the tree comprises at least 3 nodes. In a preferred embodiment, the tree comprises at least 4 nodes. In a preferred embodiment, the tree comprises at least 5 nodes.

In one embodiment, the diagnostic methods of the invention comprise averaging the predictions of one or more statistical tree models applied to the metagenes values, wherein each model includes one or more nodes, each node representing a metagene or a clinical factor, each node including a statistical predictive probability of tumor recurrence. Accordingly, the invention provides methods that use mixed trees, where a tree may contain at least two nodes, where one node represents a metagene and at least one node represents a clinical variable. In one embodiment, the clinical variables are selected from age of the subject, gender of the subject, tumor size of the sample, stage of cancer disease, histological subtype of the sample and smoking history of the subject.

In one embodiment, the statistical predictive probability is derived from a Bayesian analysis. In another embodiment, the Bayesian analysis includes a sequence of Bayes factor based tests of association to rank and select predictors that define a node binary split, the binary split including a predictor/threshold pair. Bayesian analysis is an approach to statistical analysis that is based on the Bayes law, which states that the posterior probability of a parameter p is proportional to the prior probability of parameter p multiplied by the likelihood of p derived from the data collected. This methodology represents an alternative to the traditional (or frequentist probability) approach: whereas the latter attempts to establish confidence intervals around parameters, and/or falsify a-priori null-hypotheses, the Bayesian approach attempts to keep track of how a-priori expectations about some phenomenon of interest can be refined, and how observed data can be integrated with such a-priori beliefs, to arrive at updated posterior expectations about the phenomenon. Bayesian analysis have been applied to numerous statistical models to predict outcomes of events based on available data. These include standard regression models, e.g. binary regression models, as well as to more complex models that are applicable to multi-variate and essentially non-linear data.

Another such model is commonly known as the tree model which is essentially based on a decision tree. Decision trees can be used in clarification, prediction and regression. A decision tree model is built starting with a root mode, and training data partitioned to what are essentially the “children” nodes using a splitting rule. For instance, for clarification, training data contains sample vectors that have one or more measurement variables and one variable that determines that class of the sample. Various splitting rules may be used; however, the success of the predictive ability varies considerably as data sets become larger. Furthermore, past attempts at determining the best splitting for each mode is often based on a “purity” function calculated from the data, where the data is considered pure when it contains data samples only from one clan. Most frequently, used purity functions are entropy, gini-index, and towing rule. A statistical predictive tree model to which Bayesian analysis is applied may consistently deliver accurate results with high predictive capabilities.

### (G) Treatments

In one embodiment, the diagnostic methods of the invention further comprise a therapeutic step. In one embodiment, the method comprises either administering or withholding/ceasing adjuvant therapy to the subject.

One such embodiment comprises providing adjuvant chemotherapy treatment to a subject that is predicted, based on the Lung Metagene Predictor analysis, to be at high likelihood for tumor recurrence. In one embodiment, a high likelihood of tumor recurrence corresponds to a greater than 50%, 60%, 70%, 80% or 90% chance of tumor recurrence within 1, 2, 2.5, 3, 4 or 5 years. In one embodiment, a high likelihood of tumor recurrence corresponds to a greater than 50% chance of tumor recurrence within 3 years. In another embodiment, a high likelihood of tumor recurrence corresponds to a greater than 50% chance of tumor recurrence within 5 years.

Another such embodiment comprises withholding adjuvant chemotherapy treatment to a subject that is predicted, based on the Lung Metagene Predictor analysis, to be at low likelihood for tumor recurrence. Another embodiment comprises ceasing adjuvant chemotherapy treatment to a subject that is predicted, based on the Lung Metagene Predictor analysis, to be at low likelihood for tumor recurrence. In one embodiment, a low likelihood of tumor recurrence corresponds to a lower than 50%, 40%, 30%, 20% or 10% chance of tumor recurrence within 1, 2, 2.5, 3, 4 or 5 years. In one embodiment, a low likelihood of tumor recurrence corresponds to a lower than 50% chance of tumor recurrence within 3 years. In another embodiment, a low likelihood of tumor recurrence corresponds to a lower than 50% chance of tumor recurrence within 5 years.

Adjuvant therapies suitable for use in the methods of the invention include adjuvant chemotherapies, cancer vaccines and treatment antibodies or chemotherapeutic agents. Anticancer agents that may be used include cisplatin, carboplatin, gemcitabine, paclitaxel, docetaxel, Tarceva, Iressa, and combinations thereof. Typically these would be applied after resection of the tumors. Suitable treatments for NSCLC are reviewed in the following literature: Choong et al., Clin Lung Cancer. December 2005;7 Suppl 3:S98-104; D'Amico, Semin Thorac Cardiovasc Surg. 2005 Fall;17(3):195-8; Visbal et al. Chest. October 2005;128(4):2933-43; Johnson et al. Clin Cancer Res. Jul. 1, 2005;11(13 Pt 2):5022s-5026s; Socinski et al. Clin Lung Cancer. November 2004;6(3): 162-9; and Scagliotti et al., Curr Oncol Rep. July 2003;5(4):318-25.

### III. Generation of Statistical Tree Models

Gene expression signatures that reflect the activity of a given pathway may be identified using supervised classification methods of analysis previously described (See West, M. et al. Proc Natl Acad Sci USA 98, 11462-11467 (2001). The analysis selects a set of genes whose expression levels are most highly correlated with the classification of tumor samples into tumor recurrence versus no tumor recurrence. The dominant principal components from such a set of genes then defines a relevant phenotype-related metagene, and regression models assign the relative probability of tumor recurrence.

One aspect of the invention provides methods for defining one or more statistical tree models predictive of lung tumor recurrence.

In one embodiment, the methods for defining one or more statistical tree models predictive of NSCLC tumor recurrence comprise determining the expression level of multiple genes in a set of non-small cell lung cancer samples. The samples include samples from subjects with NSCLC recurrence and samples from subjects without NSCLC recurrence. In one embodiment, at least 5, 10, 15, 20, 25, 30, 35, 40, 45, 50, 60, 70, 80, 90 or 100 samples from each of the two classes are used. The expression level of genes may be determined using any of the methods described in the preceding sections or any know in the art.

In one embodiment, the methods for defining one or more statistical tree models predictive of NSCLC tumor recurrence comprise identifying clusters of genes associated with metastasis by applying correlation-based clustering to the expression level of the genes. In one embodiment, the clusters of genes that define each metagene are identified using supervised classification methods of analysis previously described (See West, M. et al. Proc Natl Acad Sci USA 98, 11462-11467 (2001). The analysis selects a set of genes whose expression levels are most highly correlated with the classification of tumor samples into tumor recurrence versus no tumor recurrence. The dominant principal components from such a set of genes then defines a relevant phenotype-related metagene, and regression models assign the relative probability of tumor recurrence.

In one embodiment, identification of the clusters comprises screening genes to reduce the number by eliminating genes that show limited variation across samples or that are evidently expressed at low levels that are not detectable at the resolution of the gene expression technology used to measure levels. This removes noise and reduces the dimension of the predictor variable. In one embodiment, identification of the clusters comprises clustering the genes using k-means, correlated-based clustering. Any standard statistical package may be used, such as the xcluster software created by Gavin Sherlock (http://genetics.stanford.edu/˜sherlock/cluster.html). A large number of clusters may be targeted so as to capture multiple, correlated patterns of variation across samples, and generally small numbers of genes within clusters. In one embodiment, identification of the clusters comprises extracting the dominant singular factor (principal component) from each of the resulting clusters. Again, any standard statistical or numerical software package may be used for this; this analysis uses the efficient, reduced singular value decomposition function. In one embodiment, the foregoing methods comprise defining one or more metagenes, wherein each metagene is defined by extracting a single dominant value using single value decomposition (SVD) from a cluster of genes associated with NSCLC recurrence.

In one embodiment, the methods for defining one or more statistical tree models predictive of NSCLC tumor recurrence comprise defining a statistical tree model, wherein the model includes one or more nodes, each node representing a metagene, each node including a statistical predictive probability of NSCLC recurrence. This generates multiple recursive partitions of the sample into subgroups (the “leaves” of the classification tree), and associates Bayesian predictive probabilities of outcomes with each subgroup. Overall predictions for an individual sample are then generated by averaging predictions, with appropriate weights, across many such tree models. Iterative out-of-sample, cross-validation predictions are then performed leaving each tumor out of the data set one at a time, refitting the model from the remaining tumors and using it to predict the hold-out case. This rigorously tests the predictive value of a model and mirrors the real-world prognostic context where prediction of new cases as they arise is the major goal.

In one embodiment, a formal Bayes' factor measure of association may be used in the generation of trees in a forward-selection process as implemented in traditional classification tree approaches. Consider a single tree and the data in a node that is a candidate for a binary split. Given the data in this node, one may construct a binary split based on a chosen (predictor, threshold) pair (χ, τ) by (a) finding the (predictor, threshold) combination that maximizes the Bayes' factor for a split, and (b) splitting if the resulting Bayes' factor is sufficiently large. By reference to a posterior probability scale with respect to a notional 50:50 prior, Bayes' factors of 2.2, 2.9, 3.7 and 5.3 correspond, approximately, to probabilities of 0.9, 0.95, 0.99 and 0.995, respectively. This guides the choice of threshold, which may be specified as a single value for each level of the tree. Bayes' factor thresholds of around 3 in a range of analyses may be used. Higher thresholds limit the growth of trees by ensuring a more stringent test for splits.

The Bayes' factor measure will always generate less extreme values than corresponding generalized likelihood ratio tests (for example), and this can be especially marked when the sample sizes M₀ and M₁ are low. Thus the propensity to split nodes is always generally lower than with traditional testing methods, especially with lower samples sizes, and hence the approach tends to be more conservative in extending existing trees. Post-generation pruning is therefore generally much less of an issue, and can in fact generally be ignored.

Index the root node of any tree by zero, and consider the full data set of n observations, representing M_{z} outcomes with Z=z in 0, 1. Label successive nodes sequentially: splitting the root node, the left branch terminates at node 1, the right branch at node 2; splitting node 1, the consequent left branch terminates at node 3, the right branch at node 4; splitting node 2, the consequent left branch terminates at node 5, and the right branch at node 6, and so forth. Any node in the tree is labeled numerically according to its “parent” node; that is, a node j splits into two children, namely the (left, right) children (2j+1; 2j+2): At level m of the tree (m=0; 1 . . . ;) the candidates nodes are, from left to right, as 2^{m}_{—}1; 2^{m}; . . . ; 2^{m+1}−2.

Having generated a “current” tree, one may run through each of the existing terminal nodes one at a time, and assess whether or not to create a further split at that node, stopping based on the above Bayes' factor criterion. A tree having m levels has some number of terminal nodes up to the maximum possible of L=2^{m+1}−2. Inference and prediction involves computations for branch probabilities and the predictive probabilities for new cases that these underlie. This can be detailed for a specific path down the tree, i.e., a sequence of nodes from the root node to a specified terminal node. First, consider a node j that is split based on a (predictor, threshold) pair labeled (χⱼ, τⱼ), (note that we use the node index to label the chosen predictor, for clarity). Extend the notation of Section 2.1 to include the subscript j indexing this node. Then the data at this node involves M₀ⱼ cases with Z=0 and M₁ⱼ cases with Z=1. Based on the chosen (predictor, threshold) pair (χⱼ, τⱼ) these samples split into cases n₀₀ⱼ, n₀₁ⱼ, n₁₀ⱼ, n₁₁ⱼ as in the table of Section 2.1, but now indexed by the node label j. The implied conditional probabilities θ_{z,τ,J}=Pr(χ_{J}≦τ_{J}|Z=z), for z=0, 1 are the branch probabilities defined by such a split (note that these are also conditional on the tree and data subsample in this node, though the notation does not explicitly reflect this for clarity). These are uncertain parameters and, following the development of Section 2.1, have specified beta priors, now also indexed by parent node j, i.e., Be(a_{τ},ⱼ, b_{τ},ⱼ). Assuming the node is split, the two sample Bernoulli setup implies conditional posterior distributions for these branch probability parameters: they are independent with posterior beta distributions

θ_{0,τ,J}˜Be(a_{τ,J}+n₀₀ⱼ, b_{τ,j}+n₁₀ⱼ) and θ_{1,xj}˜Be(α_{τ,j}+n₀₁ⱼ, b_{x,j}+n₁₁ⱼ).

These distributions allow inference on branch probabilities, and feed into the predictive inference computations as follows.

Consider predicting the response Z* of a new case based on the observed set of predictor values x*. The specified tree defines a unique path from the root to the terminal node for this new case. To predict requires that we compute the posterior predictive probability for Z*=1/0. We do this by following x* down the tree to the implied terminal node, and sequentially building up the relevant likelihood ratio defined by successive (predictor, threshold) pairs.

For example and specificity, suppose that the predictor profile of this new case is such that the implied path traverses nodes 0, 1, 4, 9, terminating at node 9. This path is based on a (predictor, threshold) pair (χ₀, τ₀) that defines the split of the root node, (χ₁, τ₁) that defines the split of node 1, and (χ₄, τ₄) that defines the split of node 4. Hence, for any specified prior probability π Pr(Z*=1), this single tree model implies that, as a function the branch probabilities, the updated probability π* is, on the odds scale, given by

$$\frac{{π}^{*}}{( 1 - {π}^{*} )} = {λ}^{*} \frac{Pr ( {Z}^{*} = 1 )}{Pr ( {Z}^{*} = 0 )}$$

The case-control design provides no information about Pr(Z*=1) so it is up to the user to specify this or examine a range of values; one useful summary is obtained by simply taking a 50:50 prior odds as benchmark, whereupon the posterior probability is π*=λ*/(1+λ*).

Prediction follows by estimating π* based on the sequence of conditionally independent posterior distributions for the branch probabilities that define it. For example, simply “plugging-in” the conditional posterior means of each θ. will lead to a plug-in estimate of λ* and hence π*. The full posterior for π* is defined implicitly as it is a function of the θ. Since the branch probabilities follow beta posteriors, it is trivial to draw Monte Carlo samples of the θ. and then simply compute the corresponding values of λ* and hence π* to generate a posterior sample for summarization. This way, we can evaluate simulation-based posterior means and uncertainty intervals for π* that represent predictions of the binary outcome for the new case.

In considering potential (predictor, threshold) candidates at any node, there may be a number with high Bayes' factors, so that multiple possible trees with difference splits at this node are suggested. With continuous predictor variables, small variations in an “interesting” threshold will generally lead to small changes in the Bayes' factor—moving the threshold so that a single observation moves from one side of the threshold to the other, for example. This relates naturally to the need to consider thresholds as parameters to be inferred; for a given predictor χ, multiple candidate splits with various different threshold values τ reflects the inherent uncertainty about τ, and indicates the need to generate multiple trees to adequately represent that uncertainty. Hence, in such a situation, the tree generation can spawn multiple copies of the “current” tree, and then each will split the current node based on a different threshold for this predictor. Similarly, multiple trees may be spawned this way with the modification that they may involve different predictors.

In problems with many predictors, this naturally leads to the generation of many trees, often with small changes from one to the next, and the consequent need for careful development of tree-managing software to represent the multiple trees. In addition, there is then a need to develop inference and prediction in the context of multiple trees generated this way. The use of “forests of trees” has recently been urged by Breiman, L., Statistical Modeling: The two cultures (with discussion), Statistical Science, 16 199-225 (2001), and our perspective endorses this. The rationale here is quite simple: node splits are based on specific choices of what we regard as parameters of the overall predictive tree model, the (predictor, threshold) pairs. Inference based on any single tree chooses specific values for these parameters, whereas statistical learning about relevant trees requires that we explore aspects of the posterior distribution for the parameters (together with the resulting branch probabilities).

Within the current framework, the forward generation process allows easily for the computation of the resulting relative likelihood values for trees, and hence to relevant weighting of trees in prediction. For a given tree, identify the subset of nodes that are split to create branches. The overall marginal likelihood function for the tree is then the product of component marginal likelihoods, one component from each of these split nodes. Continue with the notation of Section 2.1 but now, again, indexed by any chosen node j: Conditional on splitting the node at the defined (predictor, threshold) pair (χⱼ, τⱼ), the marginal likelihood component can be calculated.

The overall marginal likelihood value is the product of these terms over all nodes j that define branches in the tree. This provides the relative likelihood values for all trees within the set of trees generated. As a first reference analysis, we may simply normalize these values to provide relative posterior probabilities over trees based on an assumed uniform prior. This provides a reference weighting that can be used to both assess trees and as posterior probabilities with which to weight and average predictions for future cases.

To ascertain the success of the tree model, any out-of-sample predictive assessment via cross-validation may be conducted. Any selection of gene, metagene or clinical variables must be part of each cross-validation analysis. The results of such “feature selection” will vary each time a tumor is analyzed, and can dramatically impact on predictive accuracy. Analyses that select a set of predictors based on the entire dataset, including the individual to be predicted, in advance of predictive evaluation are inappropriate, and lead to misleadingly over-optimistic conclusions about predictive value.

In one non-limiting exemplary embodiment of generating statistical tree models, prior to statistical modeling, gene expression data is filtered to exclude probe sets with signals present at background noise levels, and for probe sets that do not vary significantly across NSCLC samples. A metagene represents a group of genes that together exhibit a consistent pattern of expression in relation to an observable phenotype. Each signature summarizes its constituent genes as a single expression profile, and is here derived as the first principal component of that set of genes (the factor corresponding to the largest singular value) as determined by a singular value decomposition. Given a training set of expression vectors (of values across metagenes) representing two biological states, a binary probit regression model may be estimated using Bayesian methods. Applied to a separate validation data set, this leads to evaluations of predictive probabilities of each of the two states for each case in the validation set. When predicting tumor recurrence from an NSCLC sample, gene selection and identification is based on the training data, and then metagene values are computed using the principal components of the training data and additional expression data. Bayesian fitting of binary probit regression models to the training data then permits an assessment of the relevance of the metagene signatures in within-sample classification, and estimation and uncertainty assessments for the binary regression weights mapping metagenes to probabilities of relative pathway status. Predictions of tumor recurrence are then evaluated, producing estimated relative probabilities—and associated measures of uncertainty—of tumor recurrence across the validation samples. Hierarchical clustering of tumor recurrence predictions may be performed using Gene Cluster 3.0. testing the null hypothesis, which is that the survival curves are identical in the overall population.

In one embodiment, the each statistical tree model generated by the methods described herein comprises 2, 3, 4, 5, 6 or more nodes. In one embodiment of the methods described herein for defining a statistical tree model predictive of NSCLC recurrence, the resulting model predicts NSCLC tumor recurrence with at least 70%, 80%, 85%, or 90% or higher accuracy. In another embodiment, the model predicts NSCLC tumor recurrence with greater accuracy than clinical variables. In one embodiment, the clinical variables are selected from age of the subject, gender of the subject, tumor size of the sample, stage of cancer disease, histological subtype of the sample and smoking history of the subject. In one embodiment, the cluster of genes that define each metagene comprise at least 3, 4, 5, 6, 7, 8, 9, 10, 12 or 15 genes. In one embodiment, the correlation-based clustering is Markov chain correlation-based clustering or K-means clustering.

### IV. Computer Systems and Software

One aspect of the invention provides a computer-readable medium having computer-readable program codes embodied therein for performing binary prediction tree modeling to predict the recurrence of NSCLC. In one embodiment, the computer-readable program codes perform functions comprising: (ii) defining the value of one or more metagenes from expression level values of multiple genes in the sample from the subject, wherein each metagene is defined by extracting a single dominant value using single value decomposition (SVD) from a cluster of genes associated with tumor recurrence; and (iii) averaging the predictions of one or more statistical tree models applied to the values of the metagenes, wherein each model includes one or more nodes, each node representing a metagene, each node including a statistical predictive probability of tumor recurrence. In one embodiment, the expression level values of the multiple genes may be supplied by the user or automatically provided by a device that measures gene expression, such as a microarray scanner/reader.

A related aspect of the invention provides a program product (i.e. software product) for use in a computer device that executes program instructions recorded in a computer-readable medium to analyze data from the expression level of genes in an NSCLC sample from a subject and predict the likelihood of cancer recurrence in the subject.

Another related aspect of the invention provides kits comprising the program product or the computer-readable medium, optionally with a computer system. In one embodiment, the program product comprises: a recordable medium; and a plurality of computer-readable instructions executable by the computer device to analyze data from the expression level of genes in a sample from a subject and predict the likelihood of cancer recurrence in the subject, and optionally to transmit the data from one location to another. Computer-readable media include, but are not limited to, CD-ROM disks (CD-R, CD-RW), DVD-RAM disks, DVD-RW disks, floppy disks and magnetic tape. One aspect of the invention provides a binary prediction tree modeling system for performing binary prediction tree modeling to predict the recurrence of NSCLC based on gene expression data from the sample of a subject. In one embodiment, the system comprising: (i) a computer; (ii) a computer-readable medium, operatively coupled to the computer, the computer-readable medium program codes performing functions comprising: (a) defining the value of one or more metagenes from expression level values of multiple genes in the sample from the subject, wherein each metagene is defined by extracting a single dominant value using single value decomposition (SVD) from a cluster of genes associated with tumor recurrence; and (b) averaging the predictions of one or more statistical tree models applied to the values of the metagenes, wherein each model includes one or more nodes, each node representing a metagene, each node including a statistical predictive probability of tumor recurrence.

A related aspect of the invention provides kits comprising the program products or computer readable mediums described herein. The kits may also optionally contain paper and/or computer-readable format instructions and/or information, such as, but not limited to, information on statistical method, DNA microarrays, on tutorials, on experimental procedures, on reagents, on related products, on available experimental data, on using kits, on literature, on cancer treatments, on cancer diagnosis, and on other information. The kits optionally also contain in paper and/or computer-readable format information on minimum hardware requirements and instructions for running and/or installing the software. The kits optionally also include, in a paper and/or computer-readable format, information on the manufacturers, warranty information, availability of additional software, technical services information, and purchasing information. The kits optionally include a video or other viewable medium or a link to a viewable format on the internet or a network that depicts the use of the use of the software, and/or use of the kits. The kits also include packaging material such as, but not limited to, styrofoam, foam, plastic, cellophane, shrink wrap, bubble wrap, paper, cardboard, starch peanuts, twist ties, metal clips, metal cans, drierite, glass, and rubber.

The analysis of array hybridization data from a sample derived from the subject, as well as the transmission of data steps, can be implemented by using one or more computer systems. Computer systems are readily available. The processing that provides the displaying and analysis of image data for example, can be performed on multiple computers or can be performed by a single, integrated computer or any variation thereof. For example, each computer operates under control of a central processor unit (CPU), such as a “Pentium” microprocessor and associated integrated circuit chips, available from Intel Corporation of Santa Clara, Calif., USA. A computer user can input commands and data from a keyboard and display mouse and can view inputs and computer output at a display. The display is typically a video monitor or flat panel display device. The computer also includes a direct access storage device (DASD), such as a fixed hard disk drive. The memory typically includes volatile semiconductor random access memory (RAM).

Each computer typically includes a program product reader that accepts a program product storage device from which the program product reader can read data (and to which it can optionally write data). The program product reader can include, for example, a disk drive, and the program product storage device can include a removable storage medium such as, for example, a magnetic floppy disk, an optical CD-ROM disc, a CD-R disc, a CD-RW disc and a DVD data disc. If desired, computers can be connected so they can communicate with each other, and with other connected computers, over a network. Each computer can communicate with the other connected computers over the network through a network interface that permits communication over a connection between the network and the computer.

The computer operates under control of programming steps that are temporarily stored in the memory in accordance with conventional computer construction. When the programming steps are executed by the CPU, the pertinent system components perform their respective functions. Thus, the programming steps implement the functionality of the system as described above. The programming steps can be received from the DASD, through the program product reader or through the network connection. The storage drive can receive a program product, read programming steps recorded thereon, and transfer the programming steps into the memory for execution by the CPU. As noted above, the program product storage device can include any one of multiple removable media having recorded computer-readable instructions, including magnetic floppy disks and CD-ROM storage discs. Other suitable program product storage devices can include magnetic tape and semiconductor memory chips. In this way, the processing steps necessary for operation can be embodied on a program product.

Alternatively, the program steps can be received into the operating memory over the network. In the network method, the computer receives data including program steps into the memory through the network interface after network communication has been established over the network connection by well known methods understood by those skilled in the art. The computer that implements the client side processing, and the computer that implements the server side processing or any other computer device of the system, can include any conventional computer suitable for implementing the functionality described herein. References to a network, unless provided otherwise, can include one or more intranets and/or the internet.

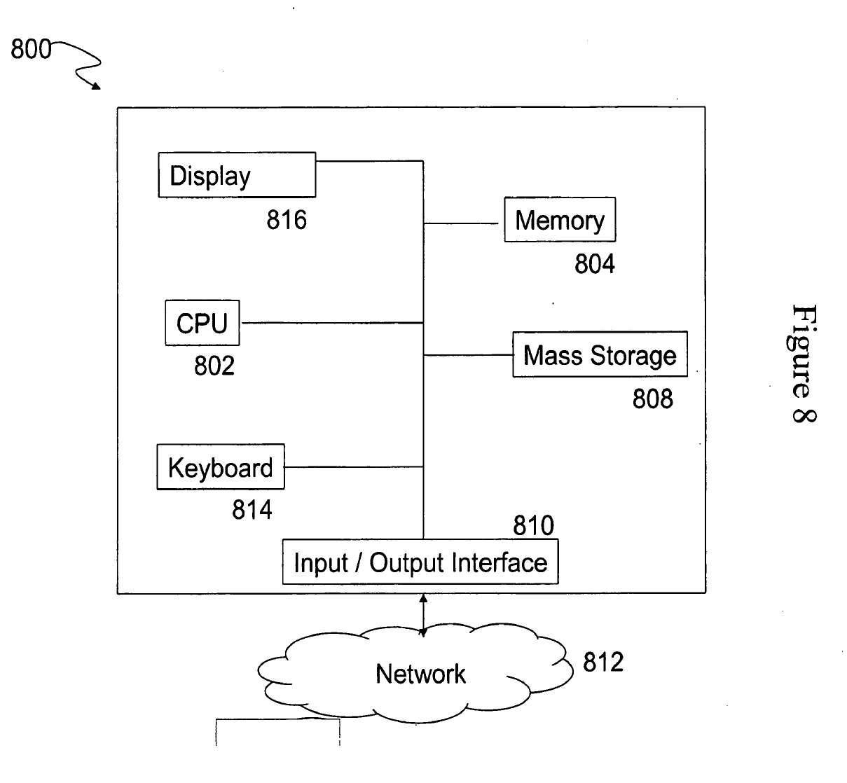

FIG. 8 shows a block diagram of a computer system 800 connected to a network 812 according to an illustrative embodiment of the invention. In one exemplary embodiment, software platforms, as well as databases, are implemented on the computer system 800. The OEMs 7, the VARs 12, and the end-customers 17 may be interconnected via network 212. The exemplary computer system 800 includes a central processing unit (CPU) 802, a memory 804, and an interconnect bus 806. The CPU 802 may include a single microprocessor or a plurality of microprocessors for configuring computer system 800 as a multi-processor system. The memory 804 illustratively includes a main memory and a read only memory. The computer 800 also includes the mass storage device 808 having, for example, various disk drives, tape drives, etc. The main memory 804 also includes dynamic random access memory (DRAM) and high-speed cache memory. In operation, the main memory 804 stores at least portions of instructions and data for execution by the CPU 802. The mass storage 808 may include one or more magnetic disk or tape drives or optical disk drives, for storing data and instructions for use by the CPU 802. At least one component of the mass storage system 808, preferably in the form of a disk drive or tape drive, stores the database used for processing. The mass storage system 808 may also include one or more drives for various portable media, such as a floppy disk, a compact disc read only memory (CD-ROM), or an integrated circuit non-volatile memory adapter (i.e. PC-MCIA adapter) to input and output data and code to and from the computer system 800.

The computer system 800 may also include one or more input/output interfaces for communications, shown by way of example, as interface 810 for data communications via the network 812. The data interface 810 may be a modem, an Ethernet card or any other suitable data communications device. The data interface 810 may provide a relatively high-speed link to a network 812, such as an intranet, internet, or the Internet, either directly or through an another external interface (not shown). The communication link to the network 812 may be, for example, optical, wired, or wireless (e.g., via satellite or cellular network). Alternatively, the computer system 800 may include a mainframe or other type of host computer system capable of Web-based communications via the network 812. The data interface 810 allows for delivering content, and accessing/receiving content via network 812.

The computer system 800 also includes suitable input/output ports or use the interconnect bus 806 for interconnection with a local display 816 and keyboard 814 or the like serving as a local user interface for programming and/or data retrieval purposes. Alternatively, server operations personnel may interact with the system 800 for controlling and/or programming the system from remote terminal devices via the network 812.

The computer system 800 may run a variety of application programs and stores associated data in a database of mass storage system 808. By way of example, the mass storage system 808 can store reference expression values or metagene compositions. The components contained in the computer system 800 are those typically found in general purpose computer systems used as servers, workstations, personal computers, network terminals, and the like. In fact, these components are intended to represent a broad category of such computer components that are well known in the art.

In one aspect, the present invention provides methods for interfacing computer technology with biological processing equipment (e.g. DNA microarray readers), including those located in a second location. In preferred embodiments, the present invention features methods for the computer to interface with equipment useful for biological processing in a remote manner. Preferably, such methods interface so as to run over a network or combination of networks such as the Internet, an internal network such as a company's own internal network, etc. thereby allowing the user to control the equipment remotely while maintaining a graphic display, updated in real time or near real time. Preferably, the methods of the present invention are used in conjunction with DNA microarray readers. In one embodiment, a computer system containing software for the prediction of tumor recurrence may interface with a DNA microarray reader at a second location, or with another computer that interfaces with the microarray reader.

### V. Diagnostic Business Methods

One aspect of the invention provides methods of conducting a diagnostic business, including a business that provide a health care practitioner with diagnostic information for the treatment of a subject afflicted with NSCLC. One such method comprises one, more than one, or all of the following steps: (i) obtaining an NSCLC sample from the subject; (ii) determining the expression level of multiple genes in the sample; (iii) defining the value of one or more metagenes from the expression levels of step (ii), wherein each metagene is defined by extracting a single dominant value using single value decomposition (SVD) from a cluster of genes associated with tumor recurrence; (iv) averaging the predictions of one or more statistical tree models applied to the values, wherein each model includes one or more nodes, each node representing a metagene or a clinical factor, each node including a statistical predictive probability of tumor recurrence; and (v) providing the health care practitioner with the prediction from step (iv).

In one embodiment, obtaining an NSCLC sample from the subject is effected by having an agent of the business (or a subsidiary of the business) such as an employee or 3rd party contractor remove an NSCLC sample from the subject, such as by a surgical procedure. In another embodiment, obtaining an NSCLC sample from the subject comprises receiving a sample from a health care practitioner, such as by shipping the sample, preferably frozen. In one embodiment, the sample is a cellular sample, such as a mass of tissue. In one embodiment, the sample comprises a nucleic acid sample, such as a DNA, cDNA, mRNA sample, or combinations thereof, that was derived from a cellular NSCLC sample from the subject. Steps (ii)-(iv) may be carried out as described in the preceding sections.

In one embodiment, the prediction from step (iv) is provided to a health care practitioner, to the patient, or to any other business entity that has contracted with the subject.

In one embodiment, the method comprises billing the subject, the subject's insurance carrier, the health care practitioner, or an employer of the health care practitioner. A government agency, whether local, state or federal, may also be billed for the services. Multiple parties may also be billed for the service.

In some embodiments, all the steps in the method are carried out in the same general location. In certain embodiments, one or more steps of the methods for conducting a diagnostic business are performed in different locations. In one embodiment, step (ii) is performed in a first location, and step (iv) is performed in a second location, wherein the first location is remote to the second location. The other steps may be performed at either the first or second location, or in other locations. In one embodiment, the first location is remote to the second location. A remote location could be another location (e.g. office, lab, etc.) in the same city, another location in a different city, another location in a different state, another location in a different country, etc. As such, when one item is indicated as being “remote” from another, what is meant is that the two items are at least in different buildings, and may be at least one mile, ten miles, or at least one hundred miles apart. In one embodiment, two locations that are remote relative to each other are at least 1, 2, 3, 4, 5, 10, 20, 50, 100, 200, 500, 1000, 2000 or 5000 km apart. In another embodiment, the two location are in different countries, where one of the two countries is the United States.

Some specific embodiments of the methods described herein where steps are performed in two or more locations comprise one or more steps of communicating information between the two locations. “Communicating” information means transmitting the data representing that information as electrical signals over a suitable communication channel (for example, a private or public network). “Forwarding” an item refers to any means of getting that item from one location to the next, whether by physically transporting that item or otherwise (where that is possible) and includes, at least in the case of data, physically transporting a medium carrying the data or communicating the data. The data may be transmitted to the remote location for further evaluation and/or use. Any convenient telecommunications means may be employed for transmitting the data, e.g., facsimile, modem, internet, etc.

In one specific embodiment, the methods comprises one or more data transmission steps between the locations. In one embodiment, the data transmission step occurs via an electronic communication link, such as the internet. In one embodiment, the data transmission step from the first to the second location comprises experimental parameter data, such as the level of gene expression of multiple genes. Other data that may be transmitted includes clinical factor data. In some embodiments, the data transmission step from the second location to the first location comprises data transmission to intermediate locations. In one specific embodiment, the method comprises one or more data transmission substeps from the second location to one or more intermediate locations and one or more data transmission substeps from one or more intermediate locations to the first location, wherein the intermediate locations are remote to both the first and second locations. In another embodiment, the method comprises a data transmission step in which a result from identifying regions of a genome is transmitted from the second location to the first location.

In one embodiment, the methods of conducting a diagnostic business comprise the step of testing the sensitivity of an NSCLC cell from the subject to a chemotherapeutic agent. Such a step may facilitate selection of a treatment plan by the health care practitioner, as not all lung cancers are expected to be treatable with equal efficacy by different therapeutic agents.

In one embodiment, the methods of conducting a diagnostic business comprise the step of determining if the subject carries an allelic form of a gene whose presence correlates to sensitivity or resistance to a chemotherapeutic agent. This may be achieved by analyzing a nucleic acid sample from the patient and determining the DNA sequence of the allele. Any technique known in the art for determining the presence of mutations or polymorphisms may be used. The method is not limited to any particular mutation or to any particular allele or gene. For example, mutations in the epidermal growth factor receptor (EGFR) gene are found in human lung adenocarcinomas and are associated with sensitivity to the tyrosine kinase inhibitors gefitinib and erlotinib. (See Yi et al. Proc Natl Acad Sci USA. May 16, 2006;103(20):7817-22; Shimato et al. Neuro-oncol. April 2006;8(2):137-44). Similarly, mutations in Breast cancer resistance protein (BCRP) modulate the resistance of cancer cells to BCRP-substrate anticancer agents (Yanase et al., Cancer Lett. Mar. 8, 2006;234(1):73-80).

### VI. Computer-Readable Media and Systems

One aspect of the invention provides a computer-readable medium comprising digitally encoded values for the composition of at least 1, 2, 3, 4, 5, 6, 7, 8, 9, 10, 15, 20, 25 or 50 metagenes, and optionally further comprising a digitally-encoded threshold value for each metagene, wherein the threshold value determines the split at a node in a statistical tree model. In one embodiment, the computer-readable medium comprises a digitally-encoded statistical predictive probability of tumor recurrence, wherein the statistical predictive probability is associated with the split at a node, in the statistical tree model, that represents the metagene. In one embodiment, the computer-readable medium contains digitally encoded values for one, two or all of (i) the composition of at least one metagenes, (ii) the threshold value defining the split at the node of a prediction tree model where the node represents the metagene; or (iii) and probabilities of cancer recurrence associated with the splits at the node.

The computer-readable medium may be a database or it may comprise values within a software program. In one embodiment, the computer-readable medium comprises a plurality of digitally-encoded values representing one or more sets of genes, wherein each set of genes corresponds to the cluster of genes defining a metagene, wherein the metagene is predictive of lung cancer recurrence in a statistical tree model. The computer readable medium may contain the gene information for one or more metagenes. For example, it may encode a first set of genes corresponding to the cluster of genes that define a first metagene, a second set of genes corresponding to the cluster of genes that define a second metagene, etc.

In one embodiment of the computer-readable medium, one of the metagenes whose value is defined by the encoded set of genes (i) is metagene 19 or (ii) shares at least 2, 3, 4, 5, 6, 7, 8, 9, 10, 11, 12 or 13 genes in common with metagene 19. Table 1 shows the cluster of genes that defines metagene 19. In one embodiment of the computer-readable medium, one of the metagenes whose value is defined by the encoded set of genes (i) is metagene 31 or (ii) shares at least 2, 3, 4, 5, 6, 7, 8, 9, 10, 11 or 12 genes in common with metagene 31. Table 2 shows the cluster of genes that defines metagene 31. In one embodiment of the computer-readable medium, one of the metagenes whose value is defined by the encoded set of genes (i) is metagene 35 or (ii) shares at least 2, 3 or 4 genes in common with metagene 35. Table 3 shows the cluster of genes that defines metagene 35. In one embodiment of the computer-readable medium, one of the metagenes whose value is defined by the encoded set of genes (i) is metagene 40 or (ii) shares at least 2, 3, 4, 5, 6, 7, 8, 9, 10, 11, 12, 13, 14, 15, 16, 17, 18, 19, 20, 21, 22, 23, 24 or 25 genes in common with metagene 40. Table 4 shows the cluster of genes that defines metagene 40. In one embodiment of the computer-readable medium, one of the metagenes whose value is defined by the encoded set of genes (i) is metagene 41 or (ii) shares at least 2, 3, 4, 5, 6, 7, 8, 9, 10, 11, 12, 13, 14 or 15 genes in common with metagene 41. Table 5 shows the cluster of genes that defines metagene 41. In one embodiment of the computer-readable medium, one of the metagenes whose value is defined by the encoded set of genes (i) is metagene 69 or (ii) shares at least 2, 3, 4, 5, 6, 7, 8, 9, 10, 11, 12 or 13 genes in common with metagene 69. Table 6 shows the cluster of genes that defines metagene 69. In one embodiment of the computer-readable medium, one of the metagenes whose value is defined by the encoded set of genes (i) is metagene 74 or (ii) shares at least 2, 3, 4, 5, 6, 7, 8, 9 or 10 genes in common with metagene 74. Table 7 shows the cluster of genes that defines metagene 74. In one embodiment of the computer-readable medium, one of the metagenes whose value is defined by the encoded set of genes (i) is metagene 79 or (ii) shares at least 2, 3, 4, 5, 6, 7, 8, 9, 10, 11, 12, 13, 14, 15, 16, 17 or 18 genes in common with metagene 79. Table 8 shows the cluster of genes that defines metagene 79. In one embodiment of the computer-readable medium, one of the metagenes whose value is defined by the encoded set of genes (i) is metagene 86 or (ii) shares at least 2, 3, 4, 5, 6, 7, 8, 9, 10, 11, 12, 13 or 14 genes in common with metagene 86. Table 9 shows the cluster of genes that defines metagene 86.

In one embodiment of the computer-readable medium, one of the metagenes whose value is defined by the encoded set of genes is metagene 19, 31, 35, 40, 41, 69, 74, 79 or 86. In another embodiment, at least two of the metagenes whose value is defined by the encoded set of genes are selected from metagenes 19, 31, 35, 40, 41, 69, 74, 79 and 86. In another embodiment, at least three of the metagenes whose value is defined by the encoded set of genes are selected from metagenes 19, 31, 35, 40, 41, 69, 74, 79 and 86. In another embodiment, at least four of the metagenes whose value is defined by the encoded set of genes are selected from metagenes 19, 31, 35, 40, 41, 69, 74, 79 and 86. In another embodiment, at least five of the metagenes whose value is defined by the encoded set of genes are selected from metagenes 19, 31, 35, 40, 41, 69, 74, 79 and 86.

In one embodiment, the computer-readable medium comprises computer-readable program codes embodied therein for performing binary prediction tree modeling to predict the recurrence of NSCLC based on gene expression data from the sample of a subject, the computer-readable medium program codes performing functions comprising: (ii) defining the value of one or more metagenes from expression level values of multiple genes in the sample from the subject, wherein each metagene is defined by extracting a single dominant value using single value decomposition (SVD) from one of the sets of genes; and (iii) averaging the predictions of one or more statistical tree models applied to the values of the metagenes, wherein each model includes one or more nodes, each node representing a metagene, each node including a statistical predictive probability of tumor recurrence.

In one aspect, the invention provides computer readable forms of the gene expression profile data of the invention, or of values corresponding to the level of expression of at least one metagene predictive or lung cancer recurrence. The metagene values may be calculated from mRNA expression levels obtained from experiments, e.g., microarray analysis. The values may also calculated from mRNA levels normalized relative to a reference gene whose expression is constant in numerous cells under numerous conditions. In other embodiments, the values in the computer are ratios of, or differences between, normalized or non-normalized mRNA levels in different samples.

The digitally-encoded data may be in the form of a table, such as an Excel table. The data may be alone, or it may be part of a larger database, e.g., comprising other metagenes, predictive tree models or clinical data. For example, the digitally-encoded data of the invention may be part of a public database. The computer readable form may be in a computer. In another embodiment, the invention provides a computer displaying the digitally-encoded data.

In one embodiment, digitally encoded values for (i) the composition of at least one metagene, (ii) the threshold value defining the split at the node of a prediction tree model where the node represents the metagene; or (iii) probabilities of cancer recurrence associated with the splits at the node, are entered into a computer system, comprising one or more databases. Instructions are provided to the computer, and the computer is capable of comparing the data entered with the data in the computer to determine whether the data entered represents a high or a low probability of cancer recurrence.

### VI. Gene Chips and Kits

Also provided are reagents and kits thereof for practicing one or more of the above described methods. The subject reagents and kits thereof may vary greatly. Reagents of interest include reagents specifically designed for use in production of the above described metagene values.

One type of such reagent is an array probe of nucleic acids, such as a DNA chip, in which the genes defining the metagenes in the cancer-recurrence predictive tree models are represented. A variety of different array formats are known in the art, with a wide variety of different probe structures, substrate compositions and attachment technologies. Representative array structures of interest include those described in U.S. Pat. Nos. 5,143,854; 5,288,644; 5,324,633; 5,432,049; 5,470,710; 5,492,806; 5,503,980; 5,510,270; 5,525,464; 5,547,839; 5,580,732; 5,661,028; 5,800,992; the disclosures of which are herein incorporated by reference; as well as WO 95/21265; WO 96/31622; WO 97/10365; WO 97/27317; EP 373 203; and EP 785 280.

The DNA chip is convenient to compare the expression levels of a number of genes at the same time. DNA chip-based expression profiling can be carried out, for example, by the method as disclosed in “Microarray Biochip Technology” (Mark Schena, Eaton Publishing, 2000). A DNA chip comprises immobilized high-density probes to detect a number of genes. Thus, the expression levels of many genes can be estimated at the same time by a single-round analysis. Namely, the expression profile of a specimen can be determined with a DNA chip. A DNA chip may comprise probes, which have been spotted thereon, to detect the expression level of the metagene-defining genes of the present invention. A probe may be designed for each marker gene selected, and spotted on a DNA chip. Such a probe may be, for example, an oligonucleotide comprising 5-50 nucleotide residues. A method for synthesizing such oligonucleotides on a DNA chip is known to those skilled in the art. Longer DNAs can be synthesized by PCR or chemically. A method for spotting long DNA, which is synthesized by PCR or the like, onto a glass slide is also known to those skilled in the art. A DNA chip that is obtained by the method as described above can be used for diagnosing a non-small cell lung cancer according to the present invention.

DNA microarray and methods of analyzing data from microarrays are well-described in the art, including in DNA Microarrays: A Molecular Cloning Manual, Ed by Bowtel and Sambrook (Cold Spring Harbor Laboratory Press, 2002); Microarrays for an Integrative Genomics by Kohana (MIT Press, 2002); A Biologist's Guide to Analysis of DNA Microarray Data, by Knudsen (Wiley, John & Sons, Incorporated, 2002); and DNA Microarrays: A Practical Approach, Vol. 205 by Schema (Oxford University Press, 1999); and Methods of Microarray Data Analysis II, ed by Lin et al. (Kluwer Academic Publishers, 2002), hereby incorporated by reference in their entirety.

One aspect of the invention provides a gene chip having a plurality of different oligonucleotides attached to a first surface of the solid support and having specificity for a plurality of genes, wherein at least 50% of the genes are common to those of metagenes 19, 31, 35, 40, 41, 69, 74, 79 and/or 86. In one embodiment, at least 70%, 80%, 90% or 95% of the genes in the gene chip are common to those of metagenes 19, 31, 35, 40, 41, 69, 74, 79 and/or 86.

One aspect of the invention provides a kit comprising: (a) any of the gene chips described herein; and (b) a computer-readable medium having computer-readable program codes embodied therein for performing binary prediction tree modeling to predict the recurrence of NSCLC based on gene expression data from the sample of a subject, the computer-readable medium program codes performing functions comprising: (ii) defining the value of one or more metagenes from expression level values of the plurality of genes, wherein each metagene is defined by extracting a single dominant value using single value decomposition (SVD) from a cluster of genes associated with tumor recurrence; and (iii) averaging the predictions of one or more statistical tree models applied to the values of the metagenes, wherein each model includes one or more nodes, each node representing a metagene, each node including a statistical predictive probability of tumor recurrence.

In some embodiments, the arrays include probes for at least 2, 3, 4, 5, 6, 7, 8, 9, 10, 15, 20, 25, 30, 40, or 50 of the genes listed in tables 1-9. In certain embodiments, the number of genes that are from the relevant tables that are represented on the array is at least 5, at least 10, at least 25, at least 50, at least 75 or more, including all of the genes listed in the appropriate table. Where the subject arrays include probes for additional genes not listed in the tables, in certain embodiments the number % of additional genes that are represented does not exceed about 50%, 40%, 30%, 20%, 15%, 10%, 8%, 6%, 5%, 4%, 3%, 2% or 1%. In some embodiments a great majority of genes in the collection are genes that define metagenes in the cancer-recurrence predictive tree models, where by great majority is meant at least about 75%, usually at least about 80% and sometimes at least about 85, 90, 95% or higher, including embodiments where 100% of the genes in the collection are metagene-defining genes. In some embodiments, at least one of the genes represented on the array is a gene whose function does not readily implicate it in cancer recurrence.

The kits of the subject invention may include the above described arrays. The kits may further include one or more additional reagents employed in the various methods, such as primers for generating target nucleic acids, dNTPs and/or rNTPs, which may be either premixed or separate, one or more uniquely labeled dNTPs and/or rNTPs, such as biotinylated or Cy3 or Cy5 tagged dNTPs, gold or silver particles with different scattering spectra, or other post synthesis labeling reagent, such as chemically active derivatives of fluorescent dyes, enzymes, such as reverse transcriptases, DNA polyrnerases, RNA polymerases, and the like, various buffer mediums, e.g. hybridization and washing buffers, prefabricated probe arrays, labeled probe purification reagents and components, like spin colurnns, etc., signal generation and detection reagents, e.g. streptavidin-alkaline phosphatase conjugate, chemifluorescent or chemiluminescent substrate, and the like.

In addition to the above components, the subject kits will further include instructions for practicing the subject methods. These instructions may be present in the subject kits in a variety of forms, one or more of which may be present in the kit. One form in which these instructions may be present is as printed information on a suitable medium or substrate, e.g., a piece or pieces of paper on which the information is printed, in the packaging of the kit, in a package insert, etc. Yet another means would be a computer readable medium, e.g., diskette, CD, etc., on which the information has been recorded. Yet another means that may be present is a website address which may be used via the internet to access the information at a removed site. Any convenient means may be present in the kits.

The kits also include packaging material such as, but not limited to, ice, dry ice, styrofoam, foam, plastic, cellophane, shrink wrap, bubble wrap, paper, cardboard, starch peanuts, twist ties, metal clips, metal cans, drierite, glass, and rubber (see products available from www.papermart.com. for examples of packaging material).

### Exemplification

The invention now being generally described, it will be more readily understood by reference to the following examples, which are included merely for purposes of illustration of certain aspects and embodiments of the present invention and are not intended to be limiting in any way.

The contents of any patents, patent applications, patent publications, or scientific articles referenced anywhere in this application are herein incorporated by reference in their entirety.

The following experimental procedures were used in the Examples.

### Patients and Tumor Samples.

A total of 198 samples from three different patient cohorts were used in our analyses. The training cohort represented 89 tumor samples from patients enrolled through the Duke Lung Cancer Prognostic Laboratory. The independent validation cohorts included samples from patients with NSCLC collected in two multicenter cooperative group trials, 25 samples from the ACOSOG Z0030 study and 84 from the prospective CALGB 9761 trial. Table 10 provides a summary of the clinical and demographic characteristics of the patients enrolled in the training (Duke), and validation (ACOSOG and CALGB) cohorts.

TABLE 10
Baseline clinical and demographic characteristics of patients in the
training (Duke) and validation cohorts (ACOSOG Z0030 and
CALGB 9761).

| | Duke Cohort | | ACOCOG Z0030 | | CALGB 9761 | |
|---|---|---|---|---|---|---|
| | ‘Training set’ | | ‘Validation set’ | | ‘Validation set’ | |
| Characteristic | (n = 89) | | (n = 25) | | (n = 84) | |
| Age (yr) | | | | | | |
| Median (Range) | 67 | (32-83) | 66.5 | (41-80) | 66 | (33-82) |
| Mean ± SD | 65 ± 9.5 | | 65 ± 4.5 | | 65 ± 10 | |
| Gender (%) | | | | | | |
| Male* | 56 | (63) | 16 | (64) | 56 | (67) |
| Female | 33 | (37) | 9 | (36) | 28 | (33) |
| Race (%) | | | | | | |
| Caucasian | 78 | (87.6) | N/A | | N/A | |
| African-American | 8 | (9) | | | | |
| Other | 3 | (3.4) | | | | |
| Tobacco | | | | | | |
| History (yr) | | | | | | |
| Never Smokers | 7 | (8) | N/A | | N/A | |
| ≦20 | 10 | (11.2) | | | | |
| 21-50 | 36 | (40.4) | | | | |
| ≧50 | 34 | (38.2) | | | | |
| Heavy Cigar ≧40 | 2 | (2.2) | | | | |
| Cell Type (%) | | | | | | |
| Adenocarcinoma | 45 | (50.6) | 11 | (44) | 84 | (100) |
| Squamous | 44 | (49.4) | 14 | (56) | 0 | (0) |
| Stage (%) | | | | | | |
| Stage Ia | 39 | (43.8) | 5 | (20) | 24 | (30) |
| Stage Ib | 30 | (33.7) | 13 | (52) | 28 | (32) |
| Stage IIa | 4 | (4.5) | 2 | (8) | 7 | (8) |
| Stage IIb | 10 | (11) | 5 | (20) | 8 | (9.5) |
| Stage IIIa | 6 | (7) | — | | 9 | (11) |
| Stage IIIb | — | | — | | 8 | (9.5) |
| Tumor Size | 3.8 ± 2.0 Cm | | 3.2 ± 1.5 Cm | | 3.4 ± 2.1 Cm | |
| (Mean ± SD) | | | | | | |
| T-Stage | | | | | | |
| 1 | 37 | (41.6) | 10 | (40) | 33 | (39) |
| 2 | 49 | (55) | 14 | (56) | 38 | (45) |
| 3 | 3 | (3.4) | 1 | (4) | 5 | (6) |
| 4 | — | | — | | 8 | (10) |
| Nodal Status (%) | | | | | | |
| Negative | 66 | (74.2) | 19 | (76) | 60 | (71) |
| Positive | 23 | (25.8) | 9 | (24) | 24 | (29) |

| | | | |
|---|---|---|---|
| Accuracy of | 93% | 72% | 79% |
| the Lung | | | |
| Metagene Model** | | | |

*There were more males in the study cohorts since one of the principal sites involved was a Veterans Affairs Medical Center.
**The ACOSOG Z0030 and the CALGB datasets were predicted using the Duke cohort as the training set. The accuracy of recurrence prediction is based on a greater than 50% probability of recurrence using the Lung Metagene Model.

Complete details of the study cohorts are provided below in the following format ([Study, i.e. Duke, CALGB, or ACOSOG]-[Surg-Path/Tstg]-[Sex, i.e. “M” for male and “F” for female]-[Histology of Tumor: “A” for adenocarcinoma and “S” for squamous cell carcinoma]-[Age of patient in years]-[Stage of Tumor]-[Size of tumor]-[Nodal Stage]-[Status (A/D)].

[Duke]-[1]-[M]-[A]-[73]-[1A]-[2]-[0]-[1]; [Duke]-[2]-[F]-[A]-[43]-[1B]-[3.5]-[0]-[0]; [Duke]-[1]-[F]-[A]-[63]-[1A]-[3]-[0]-[1]; [Duke]-[1]-[M]-[A]-[75]-[1A]-[1.2]-[2]-[0]; [Duke]-[2]-[F]-[A]-[68]-[3A]-[4.5]-[0]-[0]; [Duke]-[2]-[M]-[S]-[69]-[1B]-[3]-[0]-[1]; [Duke]-[1]-[F]-[S]-[57]-[1A]-[2]-[0]-[0]; [Duke]-[1]-[M]-[A]-[55]-[1A]-[1.8]-[0]-[0]; [Duke]-[2]-[M]-[S]-[64]-[1B]-[4]-[0]-[1]; [Duke]-[2]-[F]-[S]-[47]-[1B]-[4]-[1]-[1]; [Duke]-[2]-[F]-[A]-[67]-[2B]-[3]-[0]-[1]; [Duke]-[1]-[F]-[A]-[75]-[1A]-[2.5]-[0]-[0]; [Duke]-[2]-[M]-[A]-[73]-[1B]-[3.2]-[1]-[1]; [Duke]-[2]-[M]-[A]-[70]-[2B]-[4.8]-[0]-[1]; [Duke]-[2]-[M]-[S]-[73]-[1B]-[4]-[1]-[1]; [Duke]-[2]-[M]-[A]-[56]-[2B]-[4.5]-[1]-[1]; [Duke]-[1]-[M]-[A]-[65]-[2A]-[3]-[0]-[1]; [Duke]-[1]-[M]-[A]-[66]-[1A]-[2]-[0]-[0]; [Duke]-[2]-[M]-[S]-[58]-[1B]-[5.5]-[0]-[1]; [Duke]-[1]-[M]-[S]-[79]-[1A]-[2.5]-[0]-[0]; [Duke]-[2]-[F]-[S]-[66]-[1B]-[4.5]-[0]-[0]; [Duke]-[2]-[M]-[S]-[76]-[1B]-[4.5]-[0]-[1]; [Duke]-[2]-[M]-[A]-[71]-[1B]-[6.5]-[2]-[0]; [Duke]-[2]-[M]-[A]-[67]-[3A]-[6.5]-[1]-[1]; [Duke]-[2]-[M]-[S]-[55]-[2B]-[5]-[2]-[1]; [Duke]-[1]-[F]-[A]-[79]-[3A]-[2]-[2]-[1]; [Duke]-[2]-[M]-[S]-[81]-[3A]-[3]-[0]-[1]; [Duke]-[1]-[M]-[A]-[83]-[1A]-[1.2]-[0]-[0]; [Duke]-[1]-[M]-[A]-[62]-[1A]-[2]-[0]-[0]; [Duke]-[1]-[F]-[A]-[66]-[1A]-[3]-[0]-[1]; [Duke]-[1]-[M]-[S]-[60]-[1A]-[2.5]-[1]-[1]; [Duke]-[2]-[M]-[S]-[68]-[[1B]-[5]-[0]-[0]; [Duke]-[1]-[F]-[S]-[83]-[1A]-[1.8]-[1]-[0]; [Duke]-[1]-[M]-[S]-[72]-[2A]-[2.5]-[0]-[1]; [Duke]-[2]-[M]-[S]-[55]-[1B]-[6.8]-[0]-[0]; [Duke]-[2]-[F]-[A]-[69]-[1B]-[1.5]-[0]-[0]; [Duke]-[2]-[M]-[A]-[50]-[1B]-[4]-[0]-[1]; [Duke]-[1]-[F]-[A]-[68]-[1A]-[2.2]-[1]-[0]; [Duke]-[2]-[F]-[S]-[[55]-[2B]-[3]-[1]; [CALGB9761]-[87290]-[F]-[A]-[72]-[1b]-[T2N0]-[0]; [CALGB9761]-[78918]-[M]-[A]-[67]-[1b]-[T2N0]-[0]; [CALGB9761]-[83787]-[M]-[A]-[62]-[1b]-[T2N0]-[0]; [CALGB9761]-[85152]-[F]-[A]-[66]-[1b]-[T2N0]-[1]; [CALGB9761]-[86281]-[M]-[A]-[33]-[2b]-[T2N1]-[1]; [CALGB9761]-[79124]-[M]-[A]-[62]-[3b]-[T4N0]-[1]; [CALGB9761]-[79124]-[M]-[A]-[69]-[3b]-[T4N0]-[1]; [CALGB9761]-[83790]-[M]-[A]-[65]-[1a]-[T1N0]-[0]; [CALGB9761]-[87135]-[M]-[A]-[55]-[1b]-[T2N0]-[0]; [CALGB9761]-[86011]-[M]-[A]-[77]-[1a]-[T1N0]-[1]; [CALGB9761]-[79525]-[M]-[A]-[53]-[2b]-[T2N1]-[1]; [CALGB9761]-[78503]-[F]-[A]-[43]-[1b]-[T2N0]-[0]; [CALGB9761]-[79189]-[F]-[A]-[64]-[1a]-[T1N0]-[0]; [CALGB9761]-[79176]-[F]-[A]-[59]-[3a]-[T2N2]-[1]; [CALGB9761]-[87255]-[F]-[A]-[52]-[3b]-[T4N0]-[0]; [CALGB9761]-[82247]-[M]-[A]-[63]-[1b]-[T2N0]-[0]; [CALGB9761]-[79629]-[F]-[A]-[1a]-[T1N0]-[0]; [CALGB9761]-[83505]-[F]-[A]-[55]-[1a]-[T1N0]-[0]; [CALGB9761]-[83057]-[F]-[A]-[70]-[1a]-[T1N0]-[0]; [CALGB9761]-[77996]-[F]-[A]-[53]-[1a]-[T1N0]-[0]; [CALGB9761]-[77960]-[M]-[A]-[60]-[1a]-[T1N0]-[0]; [CALGB9761]-[78290]-[F]-[A]-[76]-[1b]-[T2N0]-[0]; [CALGB9761]-[78328]-[F]-[A]-[66]-[1a]-[T1N0]-[0]; [CALGB9761]-[77946]-[M]-[A]-[51]-[1a]-[T1N0]-[1]; [CALGB9761]-[77738]-[F]-[A]-[70]-[3a]-[T2N2]-[1]; [CALGB9761]-[78119]-[M]-[A]-[73]-[1a]-[T1N0]-[1]; [CALGB9761]-[70592]-[F]-[A]-[78]-[1b]-[T2N0]-[0]; [CALGB[9761]-[70888]-[F]-[A]-[57]-[3b]-[T4N0]-[1]; [CALGB9761]-[73916]-[F]-[A]-[70]-[1a]-[T1N0]-[0]; [CALGB9761]-[71789]-[F]-[A]-[82]-[2b]-[T2N1]-[0]; [CALGB9761]-[71621]-[F]-[A]-[82]-[3b]-[T4N0]-[0]; [CALGB9761]-[77059]-[M]-[A]-[77]-[1a]-[T1N0]-[0]; [CALGB9761]-[69314]-[M]-[A]-[77]-1b]-[T2N0]-[1]; [CALGB9761]-[77556]-[M]-[A]-[78]-[1b]-[T2N0]-[0]; [CALGB9761]-[77430]-[F]-[A]-[70]-[1a]-[T1N0]-[0]; [CALGB9761]-[68864]-[F]-[A]-[73]-[1b]-[T2N0]-[1]; [CALGB9761]-[76295]-[F]-[A]-[60]-[1b]-[T2N0]-[0]; [CALGB9761]-[71886]-[M]-[A]-[49]-[1a]-[T1N0]-[1]; [CALGB9761]-[70719]-[F]-[A]-[75]-[1a]-[T1N0]-[0]; [CALGB9761]-[69914]-[F]-[A]-[58]-[1b]-[T2N0]-[0]; [CALGB9761]-[75704]-[F]-[A]-[68]-[2a]-[T1N1]-[1]; [CALGB9761]-[70709]-[F]-[A]-[48]-[1a]-[T1N0]-[0]; [CALGB9761]-[70160]-[F]-[A]-[63]-[1b]-[T2N0]-[0]; [CALGB9761]-[74083]-[M]-[A]-[82]-[3a]-[T2N2]-[1]; [CALGB9761]-[69526]-[M]-[A]-[1a]-[T1N0]-[1]; [CALGB9761]-[.]-[M]-[A]-[58]-[1b]-[T2N0]-[1]; [CALGB9761]-[.]-[M]-[A]-[78]-[3a]-[T3N1]-[1]; [CALGB9761]-[.]-[M]-[A]-[76]-[1b]-[T2N0]-[1]; [CALGB9761]-[.]-[M]-[A]-[70]-[3b]-[T4N0]-[0]; [CALGB9761]-[.]-[M]-[A]-[62]-[1b]-[T2N0]-[0]; [CALGB9761]-[.]-[M]-[A]-[72]-[1b]-[T2N0]-[1]; [CALGB9761]-[.]-[M]-[A]-[68]-[1a]-[T1N0]-[1]; [CALGB9761]-[.]-[M]-[A]-[63]-[1a]-[T1N0]-[0]; [CALGB9761]-[.]-[M]-[A]-[58]-[2b]-[T2N1]-[1]; [CALGB9761]-[.]-[M]-[A]-[77]-[3a]-[T3N1]-[1]; [CALGB9761]-[.]-[M]-[A]-[60]-[2a]-[T1N1]-[0]; [CALGB9761]-[.]-[M]-[A]-[77]-[1b]-[T2N0]-[0]; [CALGB9761]-[.]-[M]-[A]-[78]-[1a]-[T1N0]-[1]; [CALGB9761]-[.]-[M]-[A]-[49]-[2a]-[T1N1]-[0]; [CALGB9761]-[.]-[M]-[A]-[69]-[1b]-[T2N0]-[1]; [CALGB9761]-[.]-[M]-[A]-[67]-[2a]-[T1N1]-[1]; [CALGB9761]-[.]-[A]-[68]-[3a]-[T3N1]-[0]; [CALGB9761]-[.]-[M]-[A]-[46]-[2b]-[T3N0]-[0]; [CALGB9761]-[.]-[M]-[A]-[65]-[1a]-[T1N0]-[0]; [CALGB9761]-[.]-[M]-[A]-[46]-[2b]-[T2N1]-[1]; [CALGB9761]-[.]-[M]-[A]-[65]-[1a]-[T1N0]-[1]; [CALGB9761]-[.]-[M]-[A]-[75]-[2b]-[T2N1]-[1]; [CALGB9761]-[.]-[M]-[A]-[57]-[3b]-[T4N0]-[1]; [CALGB9761]-[.]-[M]-[A]-[72]-[1b]-[T2N0]-[0]; [CALGB9761]-[.]-[M]-[A]-[64]-[3a]-[T3N1]-[1]; [CALGB9761]-[.]-[M]-[A]-[59]-[1b]-[T2N0]-[0]; [CALGB9761]-[.]-[M]-[A]-[65]-[1b]-[T2N0]-[0]; [CALGB9761]-[.]-[M]-[A]-[69]-[1a]-[T1N0]-[0]; [CALGB9761]-[.]-[M]-[A]-[77]-[1b]-[T2NO]-[1]; [CALGB9761]-[.]-[M]-[A]-[67]-[2b]-[T2N1]-[0]; [CALGB9761]-[86908]-[F]-[A]-[43]-[2a]-[T1N1]-[0]; [CALGB9761]-[76021]-[F]-[A]-[67]-[2a]-[T1N1]-[0]; [CALGB9761]-[82902]-[M]-[A]-[50]-[2a]-[T1N1]-[1]; [CALGB9761]-[.]-[M]-[A]-[67]-[1b]-[T2N0]-[0]; [CALGB9761]-[.]-[M]-[A]-[74]-[3a]-[T2N2]-[1]; [CALGB9761]-[.]-[M]-[A]-[68]-[3b]-[T4N2]-[0]; [CALGB9761]-[.]-[M]-[A]-[67]-[3a]-[T1N2]-[1]; [CALGB9761]-[.]-[M]-[A]-[57]-[1a]-[T1N0]-[1]; [CALGB9761]-[.]-[M]-[A]-[1b]-[T2N0]-[1]; [ACOSOGZ0030]-[4832]-[M]-[S]-[51]-[1B]-[2.5]-[1]; [ACOSOGZ0030]-[5377]-[M]-[S]-[66]-[1A]-[3]-[0]; [ACOSOGZ0030]-[4165]-[F]-[S]-[68]-[1B]-[4.5]-[1]; [ACOSOGZ0030]-[4305]-[F]-[A]-[48]-[2B]-[6]-[0]; [ACOSOGZ0030]-[4030]-[M]-[S]-[74]-[1B]-[3.6]-[0]; [ACOSOGZ0030]-[2679]-[M]-[S]-[65]-[1A]-[1.7]-[0]; [ACOSOGZ0030]-[5739]-[M]-[S]-[73]-[1B]-[4]-[0]; [ACOSOGZ0030]-[5107]-[F]-[S]-[47]-[1B]-[4]-[1]; [ACOSOGZ0030]-[5083]-[M]-[A]-[53]-[1B]-[5.5]-[0]; [ACOSOGZ0030]-[5273]-[M]-[S]-[68]-[1B]-[5]-[0]; [ACOSOGZ0030]-[7299]-[M]-[A]-[65]-[1B]-[3.5]-[0]; [ACOSOGZ0030]-[9209]-[F]-[S]-[57]-[2A]-[2.5]-[1]; [ACOSOGZ0030]-[9971]-[M]-[A]-[80]-[2B]-[5]-[1]; [ACOSOGZ0030]-[9808]-[F]-[S]-[75]-[1B]-[6.1]-[1]; [ACOSOGZ0030]-[6724]-[F]-[A]-[73]-[1B]-[3.9]-[0]; [ACOSOGZ0030]-[6920]-[M]-[S]-[65]-[1A]-[2.5]-[1]; [ACOSOGZ0030]-[9341]-[M]-[A]-[47]-[1B]-[3.2]-[1]; [ACOSOGZ0030]-[6962]-[M]-[S]-[51]-[1B]-[3.5]-[0]; [ACOSOGZ0030]-[8662]-[F]-[A]-[70]-[2B]-[4.8]-[1]; [ACOSOGZ0030]-[9100]-[M]-[A]-[56]-[2B]-[4.5]-[1]; [ACOSOGZ0030]-[9452]-[M]-[A]-[65]-[2A]-[3]-[1]; [ACOSOGZ0030]-[6800]-[M]-[A]-[70]-[2B]-[6]-[0]; [ACOSOGZ0030]-[4216]-[M]-[A]-[66]-[1A]-[2]-[1]; [ACOSOGZ0030]-[8250]-[F]-[S]-[41]-[1A]-[7.3]-[1]; [ACOSOGZ0030]-[6967]-[F]-[S]-[64]-[1B]-[6.7]-[0].

The initial analysis used 91 tumor samples of patients with early stage (Ia/Ib, IIa/IIb and IIIa) NSCLC, who also had clearly defined clinical outcome data, identified from the Duke Lung Cancer Prognostic Laboratory. We determined the percentage tumor content and histological type of each tumor before RNA extraction. Of the 91 RNA samples, 89 were of sufficient quality for gene expression analysis. Our initial goal was to identify gene expression patterns characteristic of certain patient cohorts within the group. The cohort of patients with early-stage NSCLC was selected to have an equal mix of the two major histological subtypes: squamous cell carcinoma and adenocarcinoma. In addition, each histologic subset had approximately equal number of patients who survived over 5 years and those who died within 2.5 years of initial diagnosis of a documented disease recurrence.

The ACOSOG Z0030 study is a completed prospective, multi-institutional phase III trial of 1100 patients with stage I NSCLC randomized to complete resection with mediastinal lymph node dissection or sampling. A subset of 416 patients had fresh-frozen tumor collected and banked at ACOSOG Central Specimen Bank at Washington University. Forty samples from patients with at least 28 months of follow-up were obtained for RNA extraction and microarray analysis. Of these, 25 cases were found to have both acceptable tumor cell content and adequate RNA quality for analysis. Approximately half (n=13) of these patients had died of cancer recurrence.

The CALGB 9761 study is a completed multi-institutional prospective phase II trial of approximately 500 patients with clinical stage I and II NSCLC, and was designed to assess the prognostic significance of micrometastatic disease using RT-PCR assay of expression of mucin-1 and carcinoembryonic antigen. Patients had fresh-frozen tumor and lymph nodes collected according to a rigorous, quality-controlled protocol such that high quality RNA was extracted from over 90% of tumors. The RNA samples derived from tumors of 84 patients were analyzed by microarray analysis (using Affymetrix U133A GeneChip). This was a blinded external validation step: the gene expression-based predictions of recurrence were made without a priori knowledge of the outcome and were independently validated with clinical outcome (survival) by a CALGB statistician. The mean follow-up for patients in this group was 5.3 years. There were 34 patients with recurrence, and 50 patients who were disease free at the time of follow-up. None of the patients in the Duke, ACOSOG, and CALGB cohorts received adjuvant chemotherapy or external beam radiation. Table 10 provides a summary of the clinical and demographic characteristics of the patients enrolled in the training (Duke), and validation (ACOSOG and CALGB) cohorts.

Histopathologic evaluation. In each of the cohorts, a single pathologist reviewed all slides for histopathologic evaluation according to WHO criteria, including adenocarcinoma subtype, degree of differentiation, lymphatic invasion, and vascular invasion. Only samples with tumor cell content greater 50% were used for the analysis.

Tumor analyses. Approximately 30 mg of lung cancer tissue was added to a chilled BioPulverizer H tube [Bio101 Systems, Carlsbad, Calif.]. Lysis buffer from the Qiagen Rneasy Mini kit was added and the tissue homogenized for 20 seconds in a Mini-Beadbeater [Biospec Products, Bartlesville, Okla.]. Tubes were spun briefly to pellet the garnet mixture and reduce foam. The lysate was transferred to a new 1.5 ml tube using a syringe and 21 gauge needle, followed by passage through the needle 10 times to shear genomic DNA. Total RNA was extracted from tumors using the Qiagen RNeasy Mini kit. The samples from the Duke Cohort and ACOSOG Z0030 were prepared and arrayed using Affymetrix U133 plus 2.0 GeneChips at the Duke Microarray Facility, and the samples from CALGB 9761 were prepared and arrayed using Affymetrix U133A GeneChips at the University of Michigan.

Gene expression arrays. For the Duke and ACOSOG samples; total RNA extracted from the tumor tissue with RNeasy kits (Qiagen, Nalencia, Calif., USA) was assessed for quality with an Agilent 2100 Bioanalyzer (Agilent, Palo Alto, Calif., USA). Hybridization targets (probes for hybridization) were prepared from total RNA according to standard Affymetrix protocols. The amount of starting total RNA for each reaction was 10 μg. Briefly, first-strand cDNA was generated using a T7-linked oligo-dT primer, followed by second-strand synthesis. An in vitro transcription reaction was performed to generate cRNA containing biotinylated UTP and CTP, which was then chemically fragmented at 95° C. for 35 min. The fragmented, biotinylated cRNA was incubated in MES buffer (2-[N-morpholino]ethansulfonic acid) containing 0.5 mg/ml acetylated bovine serum albumin to the Affymetrix GeneChip Human U133 plus 2.0 arrays at 45° C. for 16 hr, according to the directions of the manufacturer. The arrays contained over 54,000 probes, representing genes. Arrays were washed and stained with streptavidin-phycoerythrin (SAPE, Molecular Probes). Signal amplification was performed using a biotinylated antistreptavidin antibody (Vector Laboratories, Burlingame, Calif.) at 3 μg/ml. This was followed by a second staining with SAPE. Normal goat IgG (2 mg/ml) was used as a blocking agent. Scans were performed with an Affymetrix GeneChip scanner and the expression value for each gene was calculated using the Affymetrix Microarray Analysis Suite (v5.0), computing the expression intensities in ‘signal’ units defined by software. Scaling factors were determined for each hybridization based on an arbitrary target intensity of 500. Scans were rejected if the scaling factor exceeded a factor of 30.

Expression was calculated using the robust multi-array average (RMA) algorithm implemented in the Bioconductor (http://www.bioconductor.org) extensions to the R statistical programming environment. RMA generates log-2 scaled measures of expression using a linear model robustly fit to background-corrected and quantile-normalized probe-level expression data and has been shown to have a better ability to detect differential expression in spike-in experiments. The probe sets were screened to remove control genes and those with a small variance and those expressed at low levels.

All raw and RMA transformed data for the Duke, ACOSOG, and CALGB datasets are deposited in the Gene Expression Omnibus (GEO) databases website (http://www.ncbi.nlm.nih.gov/geo). The GEO accession number for the databases is GSE3593. The presentation of these data comply with MIAME (minimal information about a microarray experiment) guidelines.

Statistical analysis. We carried out statistical analysis using the metagene construction and binary prediction tree analysis as previously described²⁵⁻²⁹. The initial step filtered out genes whose maximum expression did not exceed the median value of expression or did not vary more than two-fold across the samples, to remove genes with extremely low levels of expression or little variance. The remaining genes (approximately 20,700) were then used to generate a model in which a restricted set of differentially expressed genes could distinguish the comparison groups and ultimately predict recurrence. This set of genes was then further screened by computing the simple correlation between expression of each gene and the binary recurrence outcome across samples, ranking genes by the strength of correlation and then restricting the focus to the top 10% (about 2070 genes). These genes were then clustered and used to compute metagene summaries as described below.

In the leave-one-out cross-validation analyses of the Duke training data, this process of gene screening and selection was reapplied for each sample. K-means clustering was used to create groupings of genes with between 15 and 50 genes per cluster, and a single metagene expression summary was computed for each group. The metagene for a cluster of genes is the dominant singular factor (principal component), computed using a singular value decomposition of expression levels of the genes in the metagene cluster on all samples. It represents the dominant average expression pattern of the cluster across tumor samples 26. The set of metagenes and clinical factors are then used in binary classification tree analysis to recursively partition the samples into smaller subsets within which predictions of recurrence (0=5 year disease-free survival from diagnosis of recurrence, 1=death within 2.5 years from diagnosis of recurrence) are made in terms of estimated relative probabilities 27, 31, 32. The analysis computes and weighs many such trees, and integrates them to provide overall risk predictions for each individual patient. By identifying the subset of metagenes receiving the highest weight across the trees, we identified the corresponding clusters of genes that most heavily contribute to overall risk predictions 26. The dominant metagenes that constitute the final model are described in the online Supplement.

To compare the prognostic efficacy of genomic and clinical strategies, clinical variables previously shown to be of prognostic value (age, gender, tumor size, stage of disease, histologic subtype and smoking history) were treated as factors or principal components (similar to metagenes in the genomic model) in a classification tree analysis to generate a ‘clinical model’. The end result is a probability of recurrence which represents the conglomerate prognostic value of the individual clinical variables. Using Graphpad software, we computed a c-statistic (comparable to area under the curve in a receiver operated characteristic (ROC) curve when predicting binary outcomes) for the model including just the clinical variables, a c-statistic for a model that included the genomic prediction of recurrence, and a c-statistic for a model that included both clinical and genomic variables.

Accuracy of a model was defined using the 50% probability as the cut-off—an estimate for probability of recurrence>50% was classified as high risk (i.e., the model predicts recurrence). And if the model estimates a probability of recurrence<50%, the patient is classified as being at low risk for recurrence.

Simple univariate and multivariate logistic regressions for recurrence (with and without the genomic-based assessment of recurrence risk) were also computed to assess the baseline prognostic value of the individual clinical variables (age, sex, tumor size, stage of disease, histologic subtype, and smoking history) in the Duke (training), ACOSOG (validation), and CALGB (validation) cohorts. Sensitivity, specificity, positive and negative predictive values were also calculated using the 50% probability as the cut-off. Standard Kaplan-Meier mortality curves were generated for high-risk and low-risk groups of patients using GraphPad software. For the Kaplan-Meier survival analyses, the survival curves are compared using the log-rank test. This test generates a two-tailed P value testing the null hypothesis, which is that the survival curves are identical in the overall populations.

To compare the prognostic efficacy of genomic and clinical strategies, clinical variables previously shown to be of prognostic value (age, gender, tumor size, stage of disease, histologic subtype and smoking history) were treated as factors or principal components (similar to metagenes in the genomic model) in a classification tree analysis to generate a ‘clinical model’, identical to the approach used to create the genomic model. The end result is a probability of recurrence which represents the conglomerate prognostic value of the individual clinical variables. Using Graphpad software, we computed a c-statistic (comparable to area under the curve in a receiver operated characteristic (ROC) curve when predicting binary outcomes) for the model including just the clinical variables, a c-statistic for a model that included the genomic prediction of recurrence, and a c-statistic for a model that included both clinical and genomic variables.

Accuracy of a model was defined using the 50% probability as the cut-off—if the model's estimate for probability of recurrence was >50%, the patient was classified as high risk (i.e., the model predicts recurrence). And if the model estimates a probability of recurrence<50%, the patient is classified as being at low risk for recurrence.

### Example 1

### Using Gene Expression Profiles for Improved Prognosis

Table 10 provides the details of the demographic and clinical characteristics of the patient cohorts used to develop and test of the prognostic model (FIG. 1A). All patients in this study were enrolled under IRB approved protocols, after informed consent.

Lung cancer is a heterogeneous disease resulting from the acquisition of multiple somatic mutations; given this complexity, it would be surprising if a single gene expression pattern could effectively describe and ultimately predict the clinical course of the disease for individual patients. Recognizing the importance of addressing this complexity, we have previously described methods to integrate multiple forms of data, including clinical variables and multiple gene expression profiles, to build robust predictive models for the individual patient^{25, 26}. There are two critical components to this methodological approach. We first generate a collection of gene expression profiles (termed ‘metagenes’; an example of one metagene is provided in FIG. 1B) that provide the basis for building the predictive models. We use of classification and regression tree analysis to sample from these metagenes to build prognostic models that; this approach mines the multiple profiles to best predict the clinical outcome. An example tree (one of many generated in the analysis) is depicted in FIG. 1C.

Predictive accuracy was initially assessed by leave-one-out cross-validation in which the analysis is repeatedly performed—one sample is removed at each reanalysis and the recurrence probability is predicted for that one case. The entire model-building process is repeated for each prediction and thus evaluates the reproducibility of the approach. As shown in FIG. 1D, the metagene-based model predicted recurrence with an overall accuracy of 93%. Accuracy of prediction is based on a >50% probability of recurrence being consistent with recurrence and vice versa. As a measure of model stability, we generated multiple iterations of randomly split training and validation sets from within the Duke cohort and observed a >85% accuracy in prognostic capability (data not shown). The gene expression model for predicting recurrence was superior to a predictive model generated with the same methods but using only clinical data including tumor size, stage of disease, age, sex, histologic subtype and smoking history. The model built on the clinical data only had an accuracy of 64% (FIG. 1E); the model built on genomic data had an accuracy of over 90%. In addition, inclusion of the clinical data with the genomic data did not further improve the accuracy of the prediction of recurrence, over genomic data alone.

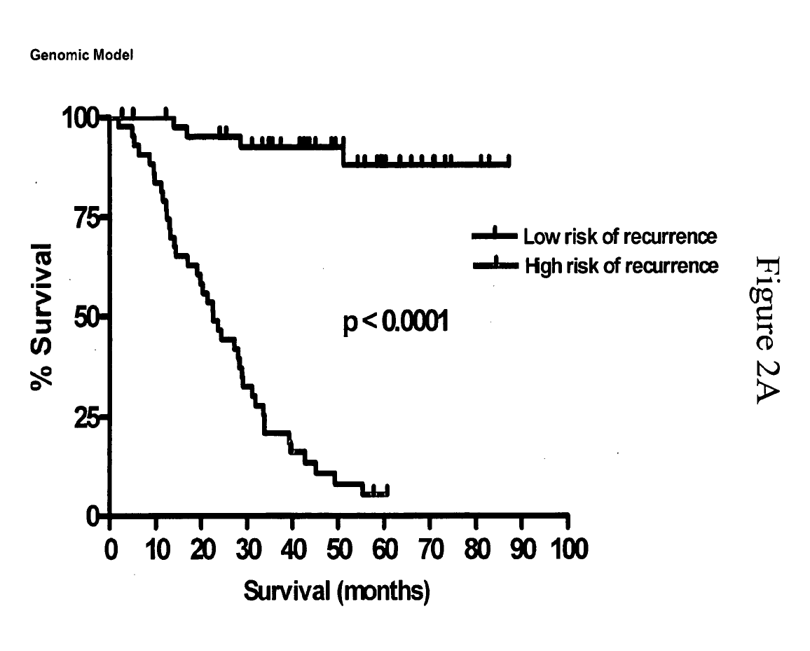

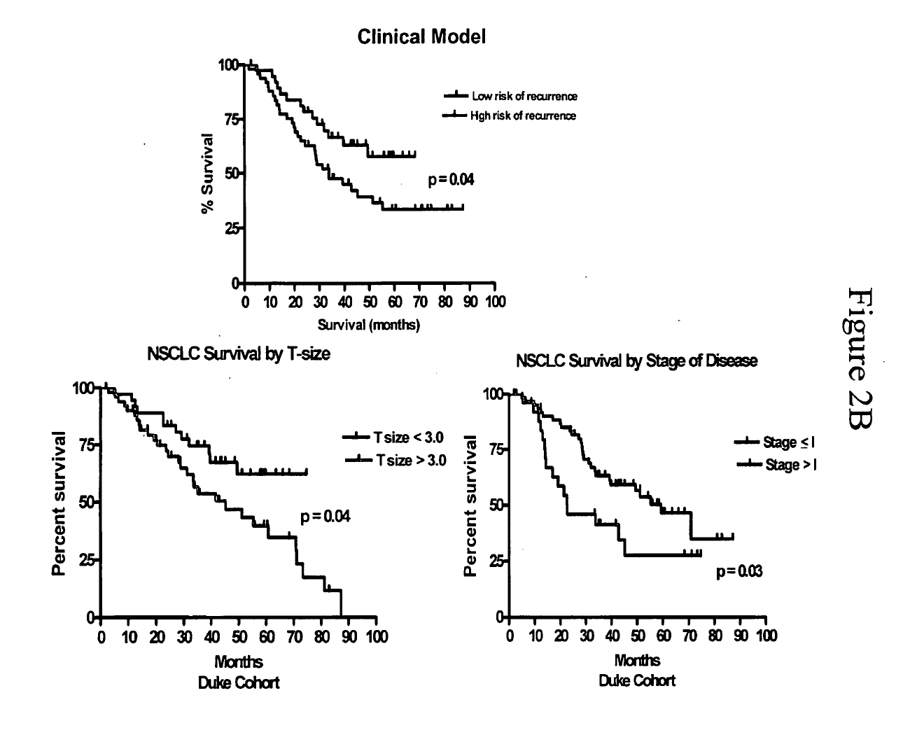

That the model based on gene expression outperformed clinical risk factors in identifying patients at risk of recurrence is also supported by Kaplan Meier analyses. Whereas the genomic-based prediction of risk identified two distinct groups of patients with respect to survival (FIG. 2A), the distinctions afforded by the clinically based predictions (we tested two models based on clinical data: one that combined all the clinical variables in a manner similar to the genomic model and the other, based on individual clinical prognostic factors (tumor size and stage)), were less clear (FIG. 2B). Univariate and multivariate analyses (with and without the genomic-based assessment of recurrence risk) to assess the relative prognostic value of the individual clinical variables (age, sex, tumor size, stage of disease, histologic subtype, and smoking history) and the metagene-based genomic model, in the Duke training cohort, as well as the two validation cohorts, showed that the genomic model performed significantly better (p<0.0001, multivariate analysis) than pathologic stage, tumor size, nodal status, age, gender, histologic subtype and smoking history (See Table in next page).

| | | | | |
|---|---|---|---|---|
| | Univariate | | Multivariate | |
| Predictor | OR [95% CI] | p-value | OR [95% CI] | p-value |

Duke Cohort (n = 89)

| | | | | |
|---|---|---|---|---|
| Age (yrs) | 1.01 [0.96, 1.05] | 0.750 | n/a | — |
| Gender: Male | 1.34 [0.56, 3.18] | 0.509 | n/a | — |
| Cell Type: Squamous cell | 1.10 [0.47, 2.53] | 0.831 | n/a | — |
| Tumor size >3 cm | 2.29 [0.98, 5.39] | 0.057 | 2.99 [0.52, 17.10] | 0.219 |
| Lymph node status: Positive | 2.47 [0.88, 6.89] | 0.085 | 4.87 [0.69, 34.33] | 0.112 |
| Pathologic stage: Non-stage I | 3.28 [1.13, 9.48] | 0.029 | 7.27 [1.02, 51.80] | 0.048 |
| Genomic prediction | 137 [29, 650] | <0.0001 | 207 [32, 1348] | <0.0001 |
| (>50% probability of recurrence) | | | | |

ACOSOG Cohort (n = 25)

| | | | | |
|---|---|---|---|---|
| Age (yrs) | 0.97 [0.89, 1.04] | 0.391 | n/a | — |
| Gender: Male | 2.57 [0.47, 14.10] | 0.277 | n/a | — |
| Cell Type: Squamous cell | 1.20 [0.25, 5.84] | 0.821 | n/a | — |
| Tumor size (>3 cm) | 0.84 [0.49, 1.44] | 0.529 | n/a | — |
| Lymph node status: Positive | 3.13 [0.47, 20.58] | 0.236 | n/a | — |
| Pathologic stage: Non-stage I | 3.13 [0.47, 20.58] | 0.236 | n/ | — |
| Genomic prediction | 35.90 [2.78, 463] | 0.006 | 35.90 [2.78, 463] | 0.006 |
| (>50% probability of recurrence) | | | | |

CALGB Cohort (n = 84)**

| | | | | |
|---|---|---|---|---|
| Age (yrs) | 1.005 [0.96, 1.05] | 0.825 | n/a | — |
| Gender: Male | 5.33 [1.87, 15.18] | 0.002 | 4.80 [1.32, 17.44] | 0.017 |
| Pathologic stage: Non-stage I | 3.14 [1.26, 7.87] | 0.014 | 4.08 [1.27, 13.09] | 0.018 |
| Lymph node status: Positive | 3.45 [1.27, 9 37] | 0.015 | 4.60 [1.32, 15.95] | 0.016 |
| Genomic prediction (>50% | 16.1 [4.78, 54.23] | <0.0001 | 16.6 [4.41, 62.76] | <0.0001 |
| probability of recurrence) | | | | |

In both the Dulce and CALGB datasets tumor size & lymph node status were analyzed separately from non-stage I variable in the multiple logistic regression analysis due to co-linearity.
**Exact tumor size data was not available for the CALGB data. All samples included in the CALGB cohort were adenocarcinomas.

Finally, further confirmation that the model represents tumor biology is seen from the observation that the metagenes that have the greatest discriminatory capability in the model include genes that have previously been shown to have clinical relevance in NSCLC. In some instances, a metagene represents a single molecular process like angiogenesis (metagene 19), a proven target for therapy in NSCLC. Other key metagenes such as metagene 41 included a mixture of biological processes as represented by RAF, PI3kinase, TP53 and Myc signaling pathways.

### Example 2

### The Metagene Prognostic Model is Valid Across Distinct Subtypes of NSCLC

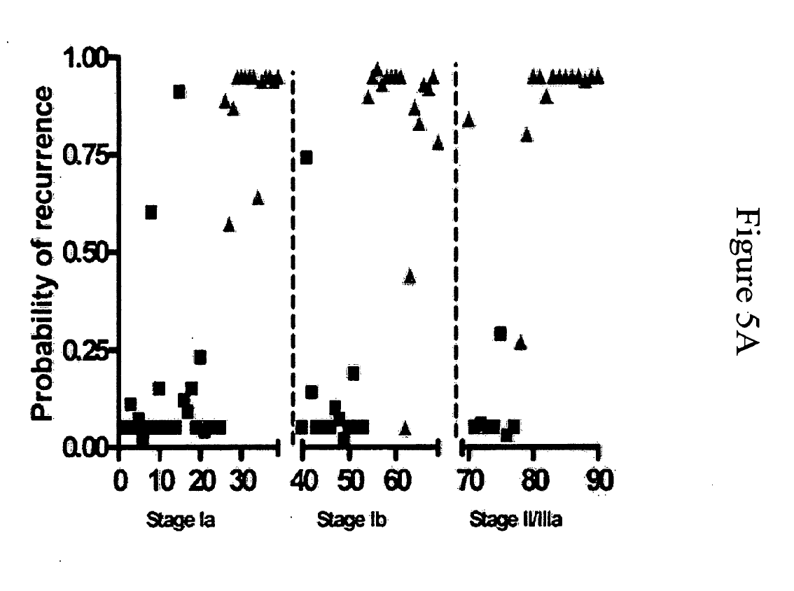

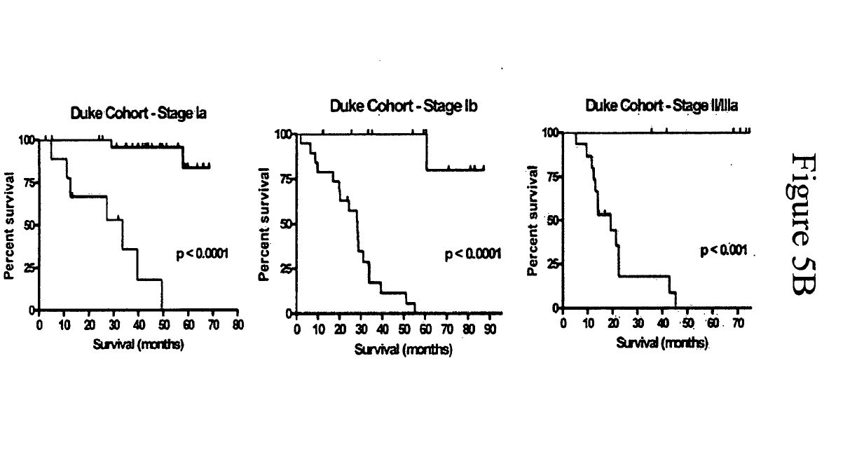

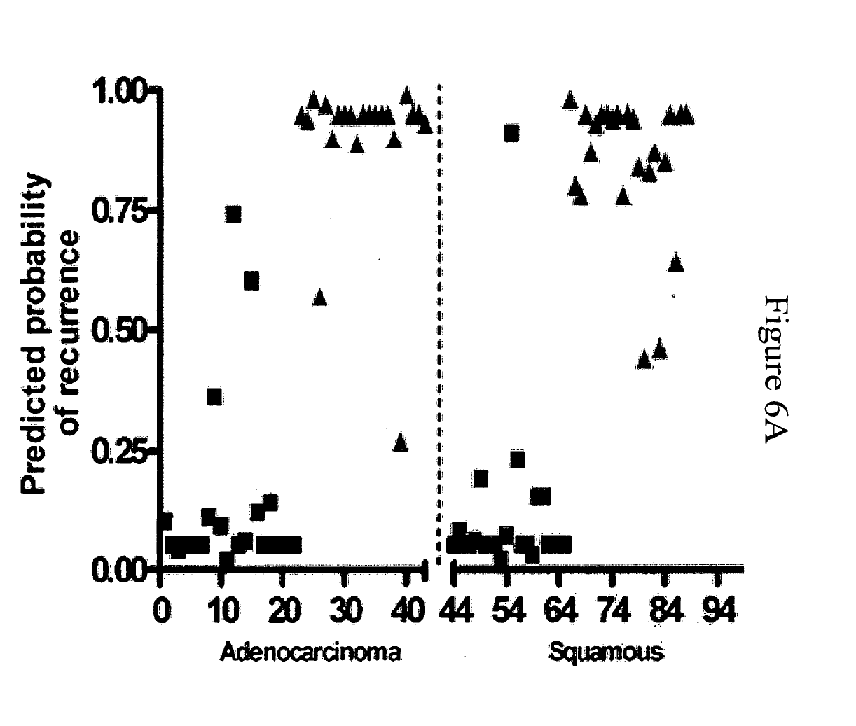

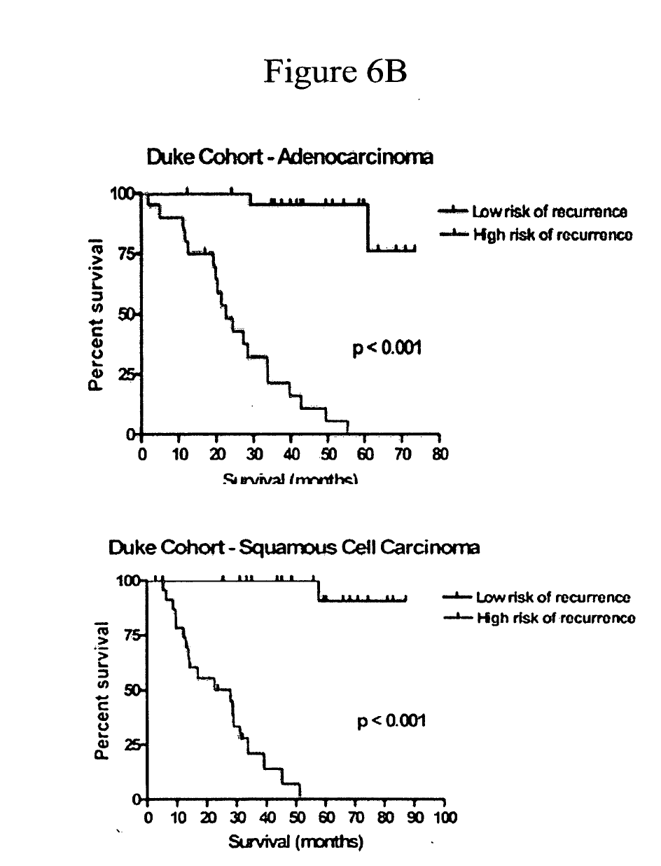

The samples used for the development of the prognostic model represented both major histological subtypes of NSCLC (adenocarcinoma and squamous cell carcinoma) as well as all early stages of disease. To assess the general robustness of the prognostic model in the Duke cohort, we examined the predictions of risk as a function of these variables. As shown in FIG. 5, the gene expression based model was consistently accurate across all of the early stages of NSCLC. This is reflected in not only the estimated risk of recurrence but also seen in Kaplan Meier survival analysis for each stage. In addition, the model was equally effective in predicting recurrence for both the common histologic types (adenocarcinoma as well as squamous cell carcinoma) and again, the Kaplan Meier curves demonstrate the prognostic value of the metagene model irrespective of histologic subtype (FIG. 5).

### Example 3

### Validation of the Metagene Prognostic Model in Two Multi-Center Cooperative Group Studies

Use of a new prognostic model to assess risk of recurrence to inform the decision of whether to use adjuvant chemotherapy requires demonstration that the model is robust when applied to independent heterogeneous populations of patients and conditions of sample acquisition. We therefore evaluated the ability of the model generated from the Duke training set to predict recurrence risk using two multi-center cooperative group studies (ACOSOG Z0030 and CALGB 9761) (FIG. 1A). These sample sets represent the full spectrum of clinical outcomes without any selection for long or short survival.

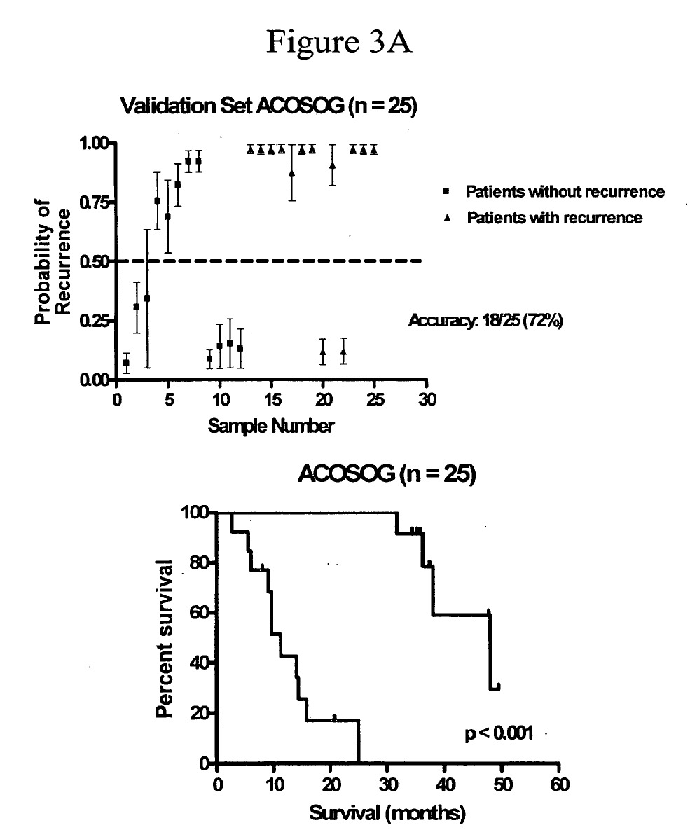

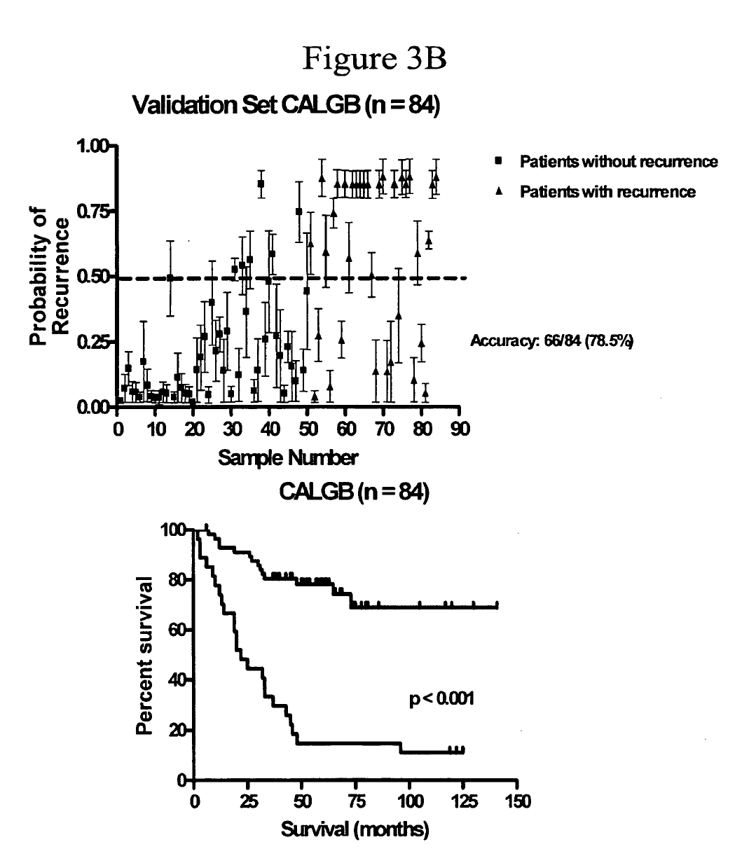

We analyzed 25 samples from the ACOSOG Z0030 trial to validate the performance of the Duke-generated predictor of recurrence. As shown in FIG. 3A, the accuracy, using a 50% probability of recurrence as a cut-off, for predicting recurrence in the ACOSOG samples was approximately 72% (sensitivity: 85%, specificity: 58%, positive predictive value: 69%, negative predictive value 78%:). This level of accuracy provides an assessment of robustness of risk predictions and is substantial, particularly given the sample heterogeneity and the fact that the clinical outcomes of patients in the ACOSOG dataset represent a prospective collection. Kaplan Meier survival curves stratified by the genomic risk predictions strongly support the reliability of the predictions (right panel). In addition, multivariate analysis shows that the patients with a genomic model estimate of >50% in the ACOSOG cohort were more likely to have disease recurrence that those with a predicted probability of <50% (adjusted odds ratio: 35.9 (95% CI: 2.78-463).

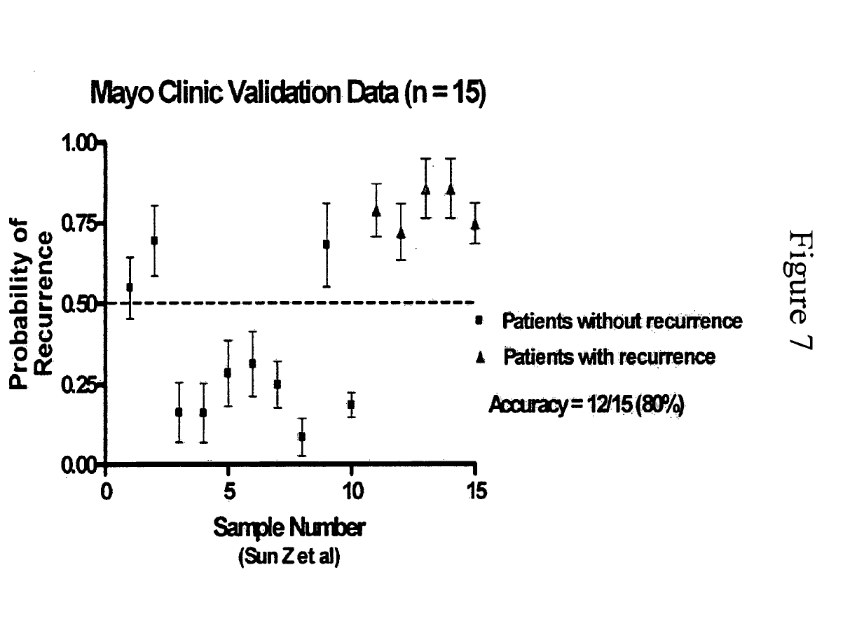

We analyzed 84 samples from the CALGB 9761 trial as a second independent validation set. The outcome of these CALGB patients was blinded to the investigators applying the predictive model; thus, the genomic predictions of recurrence were submitted to a CALGB statistician for a determination of outcome. As shown in FIG. 3B, the predictive accuracy of the model for the CALGB samples was 79% (sensitivity: 68%, specificity: 88%, positive predictive value: 79%, negative predictive value: 80%). Again, Kaplan Meier analysis showed a statistically significant difference in the survival of patients stratified according to the genomic-based prognosis model (right panel). Similar to the results seen in the Duke and ACOSOG data, the adjusted odds ratio for disease recurrence in the CALGB cohort was 16.6 (95% CI: 4.4-62.7) when model estimate for recurrence was >50%. We also applied the metagene model to another sample set of fifteen patients with surgically resected stage I squamous cell lung cancer. Using the metagene prognostic model, we were able to accurately predict the outcome (recurrence) in all five patients with recurrence, and 7/10 patients without recurrence, for an overall accuracy of 12/15 (80%) (FIG. 7).

Finally, to evaluate to what extent the genomic model adds to the clinicians' ability to estimate prognosis, we computed a C statistic as a measure of the capacity of the clinical or genomic information to discriminate patients with respect to recurrence. For the ACOSOG cohort, the C statistic based only on clinical variables was 0.67; this increased to 0.84 by inclusion of genomic data. For the CALGB cohort, the genomic data increased the C statistic from 0.73 with clinical data alone to 0.87 with the inclusion of genomic data. Clearly, the genomic data transforms a very limited clinical-based prognosis to one with substantial capacity to discriminate patients likely to recur.

### Example 4

### Application of Refined Prognosis

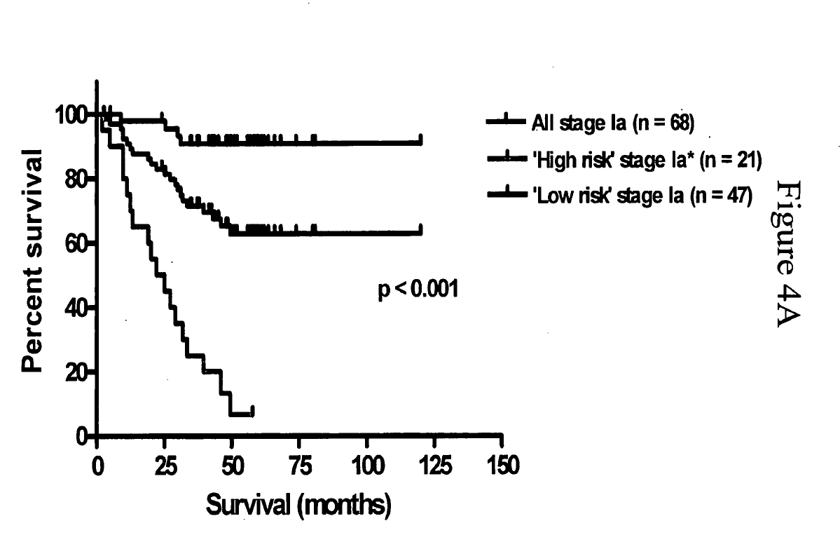

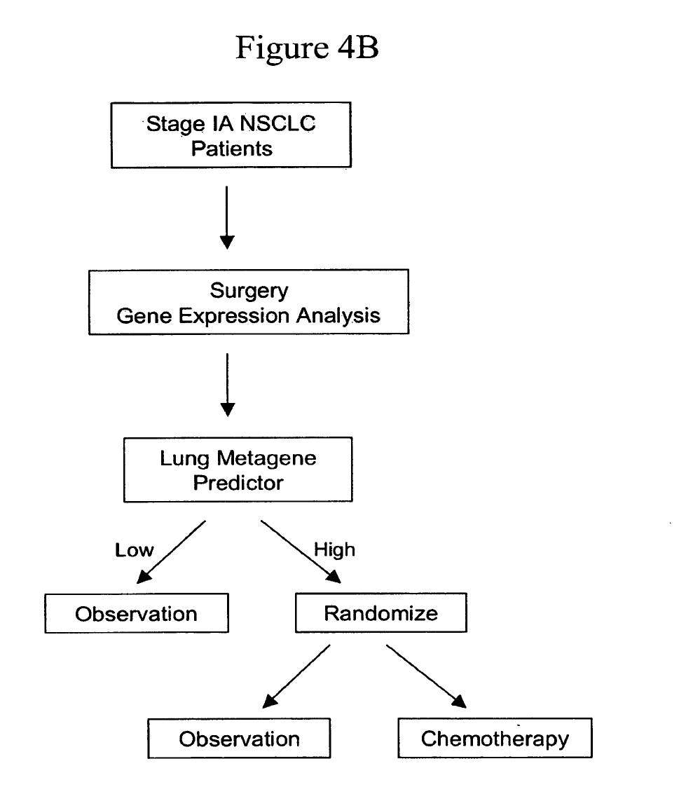

While this refinement of prognosis could go both directions (increase or decrease estimate of risk), it is more plausible to consider the use of such a tool to reclassify patients to a higher risk category. In particular, one might consider the fact that a proportion of Stage IA patients might be more appropriately categorized as ‘higher risk’ and thus candidates for chemotherapy. By way of illustration, we focused on a group of 68 patients within the Duke, ACOSOG, and CALGB cohorts that were classified clinically as Stage IA. Kaplan Meier survival curves were generated for the group as a whole as well as the subgroups predicted to be at high or low risk of recurrence based on the Duke genomic prognostic model. It is evident from the analysis in FIG. 4A that while the survival rate for Stage IA patients as a whole is approximately 70% at 4 yr (black curve), the survival rate for those Stage IA patients predicted at high risk by the metagene model (>50% probability) is less than 10% (red curve). Clearly, the designation by stage is imprecise and includes a very broad range of actual survival. The value of the genomic model is to then identify a sub-group within this heterogeneous population of patients with early stage NSCLC that might be better classified in a risk category that would be appropriate for adjuvant chemotherapy.

Although the development of gene expression profiles that can classify cancer patients with respect to risk of recurrence has been demonstrated in many instances, it is the opportunity to use an improved and refined prognostic tool to change a clinical decision that is one unique aspect of this work. In particular, the current guidelines for treatment of Stage I NSCLC patients provides an opportunity to employ an improved prognostic model to refine the current imprecise assessment of risk and the decision of who to treat, leading to a more personalized cancer treatment. In this case, the refinement of prognosis using the metagene model defines an opportunity for a prospective randomized, Phase III clinical trial that would evaluate the benefit of the identification of a sub-group of Stage IA patients estimated to be at high risk (FIG. 4B). Patients initially classified as clinical Stage IA would undergo surgery, the metagene prognostic model would be applied to then identify those individual patients predicted to be at high risk for recurrence. High risk patients would then be randomized into an observation arm (current standard of care for Stage IA patients) versus an adjuvant chemotherapy arm, to evaluate the extent to which genomic reclassification results in improved survival. We believe this represents a critical first step in the use of genomic tools as a strategy to refine prognosis and improve the selection of patients appropriate for adjuvant chemotherapy.

### REFERENCES

The following references have been cited throughout the specification and are incorporated by reference along with other cited references:
1. Spira A, Ettinger D S. Multidisciplinary management of lung cancer. N Engl J Med 2004;350(4):379-92.
2. Hoffman P C, Mauer A M, Vokes E E. Lung cancer. Lancet 2000;355:479-85.
3. Mountain C F. Revisions in the international system for staging lung cancer. Chest 1997;111:1710-7.
4. Nesbitt J C, Putnam J B, Jr., Walsh G L, Roth J A, Mountain C F. Survival in early-stage non-small cell lung cancer. Ann Thorac Surg 1995;60:466-72.
5. Mountain C F. The new international staging system for lung cancer. Surg Clin North Am 1987;67:925-35.
6. D'Amico T A, Massey M, Herndon J Ed, Moore M B, Harpole D H, Jr. A biologic risk model for Stage 1 lung cancer: Immunohistochemical analysis of 408 patients using 10 molecular markers. J Thorac Cardiovasc Surg 1999;117:736-43.
7. Brundage M D, Davies D, Mackillop W J. Prognostic factors in non-small cell lung cancer: a decade of progress. Chest 2002;122:1037-57.
8. Meyerson M, Carbone D P. Genomic and proteomic profiling of lung cancers: lung cancer classification in the age of targeted therapy. J Clin Oncol 2005;23(14):3219-26.
9. Arriagada R, Bergman B, Dunant A, et al. Cisplatin-based adjuvant chemotherapy in patients with completely resected non-small-cell lung cancer. N Engl J Med 2004;350:351-60.
10. Winton T, Livingston R, Johnson D, al e. Vinorelbine plus Cisplatin vs. observation in resected non-small cell lung cancer. N Engl J Med 2005;352:2589-97.
11. Douillard J, Rosell R, Delena M, Legroumellec A, Torres A, Carpagnano F. ANITA: Phase III adjuvant vinorelbine (N) and cisplatin (P) versus observation (OBS) in completely resected (stage I-III) non-small-cell lung cancer (NSCLC) patients (pts): Final results after 70-month median follow-up. J Clin Oncol 2005;21(14S):7013.
12. Kato H, Ichinose Y, Ohta M, et al. Japan Lung Cancer Research Group on Postsurgical Adjuvant Chemotherapy. A randomized trial of adjuvant chemotherapy with uraciltegafur for adenocarcinoma of the lung. N Engl J Med 2004;350:1713-21.
13. Strauss G M, Herndon J Ed, Maddaus M A, al. e. Randomized clinical trial of adjuvant chemotherapy with paclitaxel and carboplatin following resection in Stage 1B non-small cell lung cancer. J Clin Oncol 2004;22(14S):7019.
14. Tonon G, Wong K K, Maulik G, et al. High-resolution genomic profiles of human lung cancer. Proc Natl Acad Sci USA 2005;102(27):9625-30.
15. Schneider P M, Praeuer H W, Stoeltzing O, et al. Multiple molecular marker testing (p53, C-Ki-ras, c-erbB-2) improves estimation of prognosis in potentially curative resected non-small cell lung cancer. Br J Cancer 2000;83:473-9.
16. Berrar D, Sturgeon B, Bradbury I, Downes C S, Dubitzky W. Survival trees for analyzing clinical outcome in lung adenocarcinomas based on gene expression profiles: Identification of neogenin and diacylglycerol kinase alpha expression as critical factors. J Comput Biol 2005;12:534-44.
17. Ju Z, Kapoor M, Newton K, et al. Global detection of molecular changes reveals concurrent alteration of several biological pathways in non-small cell lung cancer cells. Mol Genet Genomics 2005;28:1-14.
18. Beer D G, Kardia S L R, Huang C C, et at. Gene-expression profiles predict survival of patients with lung adenocarcinoma. Nat Med 2002;8:816-24.
19. Chen G, Gharib T G, Wang H, et al. Protein profiles associated with survival in lung adenocarcinoma. 2003;100:13537-42.
20. Bhattacharjee A, Richards W G, Staunton J, et al. Classification of human lung carcinomas by mRNA expression profiling reveals distinct adenocarcinoma subclasses. 2001;98:13790-5.
21. Wigle D A, Jurisica I, Radulovich N, et al. Molecular profiling of non-small cell lung cancer and correlation with disease-free survival. Cancer Res 2002;62:3005-8.
22. Kikuchi T, Daigo Y, Katagiri T, et al. Expression profiles of non-small cell lung cancers on cDNA microarrays: Identification of genes for prediction of lymph-node metastasis and sensitivity to anti-cancer drugs. Oncogene 2003;22:2192-205.
23. Garber M E, Troyanskaya O G, Schluens K, et al. Diversity of gene expression in adenocarcinoma of the lung. Proc Natl Acad Sci USA 2001;8:13784-9.
24. Yanaihara N, Caplen N, Bowman E, et al. Unique microRNA molecular profiles in lung cancer diagnosis and prognosis. Cancer Cell 2006;9:189-98.
25. Pittman J, Huang E, Dressman H, et al. Models for individualized prediction of disease outcomes based on multiple gene expression patterns and clinical data. Proc Nat'l Acad Sci 2004;101:8431-6.
26. Pittman J, Huang E, Wang Q, Nevins J R, West M. Bayesian analysis of binary prediction tree models for retrospectively sampled outcomes. Biostatistics 2004;5:587-601.
27. Nevins J R, Huang E S, Dressman H, Pittman J, Huang A T, West M. Towards integrated clinic-genomic models for personalized medicine: combining gene expression signatures and clinical factors in breast cancer outcomes prediction. Hum Mol Genet 2003;12:R153-7.
28. Huang E, Cheng S H, Dressman H, et al. Gene expression predictors of breast cancer outcomes. Lancet 2003;361:1590-6.
29. West M, Blanchette C, Dressman H, et al. Predicting the clinical status of human breast cancer by using gene expression profiles. Proc Natl Acad Sci USA 2001;98:11462-7.
30. Denison D, Mallick B, Smith A F M. Biometrika 1999;85:363-77.
31. Breiman L. The two cultures. Statistical Science 2001;16:199-225.

1. A binary prediction tree modeling system for predicting the likelihood of developing tumor recurrence in a subject afflicted with non-small cell lung cancer (NSCLC), the system comprising: a computer; a computer-readable medium operatively coupled to the computer, the computer-readable medium program codes performing functions comprising the following steps:
(i) determining the expression level of multiple genes in a NSCLC sample from the subject;
(ii) defining the value of one or more metagenes from the expression levels of step (i), wherein the value of each metagene is defined by extracting a single dominant value using single value decomposition (SVD) from the cluster of genes associated with the metagene;
(iii) averaging the predictions of one or more statistical tree models applied to the values of the metagenes, wherein each model includes one or more nodes, each node representing a metagene, each node including a statistical predictive probability of tumor recurrence, thereby predicting the likelihood of developing tumor metastasis in a subject afflicted with non-small cell lung cancer (NSCLC).

2-34. (canceled)

35. The system of claim 1, wherein at least one the metagenes is metagene 19, 31, 35, 40, 41, 69, 74, 79 or 86.

36. The system of claim 1, wherein the cluster of genes corresponding to at least one of the metagenes comprises 3 or more genes in common to metagene 19, 31, 35, 40, 41, 69, 74, 79 or 86.

37. The system of claim 1, wherein the cluster of genes corresponding to at least one metagene comprises 5 or more genes in common to metagene 19, 31, 35, 40, 41, 69, 74, 79 or 86.

38. The system of claim 1, wherein the cluster of genes corresponding to at least one metagene comprises at least 10 genes, wherein half or more of the genes are common to metagene 19, 31, 35, 40, 41, 69, 74, 79 or 86.

39. The system of claim 1, wherein each cluster of genes comprises at least 3 genes.

40-41. (canceled)

42. The system of claim 1, wherein each cluster of genes comprises at least 10 genes.

43-44. (canceled)

45. The system of claim 1, wherein each cluster of genes comprises at least 20 genes.

46-47. (canceled)

48. The system of claim 1, wherein at least one of the metagenes shares at least 50% of its defining genes in common with metagene 19, 31 35, 40, 41, 69, 74, 79 or 86.

49. The system of claim 1, wherein at least one of the metagenes shares at least 75% of its defining genes in common with metagene 19, 31, 35, 40, 41, 69, 74, 79 or 86.

50-52. (canceled)

53. The system of claim 1, wherein the cluster of genes for at least two of the metagenes share at least 50% of their genes in common with one of metagenes 19, 31, 35, 40, 41, 69, 74, 79 or 86.

54. The system of claim 1, wherein the cluster of genes for at least two of the metagenes share at least 75% of their genes in common with one of metagenes 19, 31, 35, 40, 41, 69, 74, 79 or 86.

55-93. (canceled)

94. A kit comprising: (a) a gene chip having a plurality of different oligonucleotides attached to a first surface of the solid support and having specificity for a plurality of genes, wherein at least 50% of the genes are common to those of metagenes 19, 31, 35, 40, 41, 69, 74, 79 and/or 86; and (b) a computer-readable medium having computer-readable program codes embodied therein for performing binary prediction tree modeling to predict the recurrence of NSCLC based on gene expression data from the sample of a subject, the computer-readable medium program codes performing functions comprising: (ii) defining the value of one or more metagenes from expression level values of the plurality of genes, wherein each metagene is defined by extracting a single dominant value using single value decomposition (SVD) from a cluster of genes associated with tumor recurrence; (iii) averaging the predictions of one or more statistical tree models applied to the values of the metagenes, wherein each model includes one or more nodes, each node representing a metagene, each node including a statistical predictive probability of tumor recurrence.

95. A method of predicting the likelihood of developing tumor reoccurrence in a subject afflicted with non-small cell lung cancer (NSCLC) comprising:
(a) comparing the expression level of multiple genes in a NSCLC sample from a subject to one or more metagenes selected from the group consisting of metagene 19, 31, 35, 40, 41, 69, 74, 79 and 86;
(b) using the comparison of step (a) to predict the likelihood of the subject developing tumor reoccurrence.
